(19) United States
(12) Patent Application Publication
(10) Pub. No.: US 2026/0074046 A1
(43) Pub. Date: Mar. 12, 2026

(54) PALATAL EXPANSION MANAGEMENT TOOL

(71) Applicant: Align Technology, Inc., San Jose, CA (US)

(72) Inventors: Chunhua Li, Cupertino, CA (US); Roman A. Roschin, Moscow (RU); Jeeyoung Choi, Sunnyvale, CA (US); Bastien Pesenti, San Jose, CA (US); Natalia Lysaya Powers, San Jose, CA (US); Sibel Narin, Los Gatos, CA (US); Dmitry Mednikov, Moscow (RU); Anna Lukinykh, Madrid (ES); Nataliia Tokmakova, Madrid (ES); Savva Kuzmin, Toronto (CA); Elena Zhalina, Berlin (DE); Aleksandr Strizhev, Korolyov (RU); Larissa Fernandez Alfaro, San Jose, CA (US); Mitra Derakhshan, Herndon, VA (US); Che-Jui Liao, San Jose, CA (US)

(21) Appl. No.: 19/318,319

(22) Filed: Sep. 3, 2025

Related U.S. Application Data
(60) Provisional application No. 63/691,955, filed on Sep. 6, 2024.
(60) Provisional application No. 63/765,131, filed on Feb. 28, 2025.

(51) Int. Cl. G16H 20/40; A61C 7/10

(52) U.S. Cl. CPC G16H 20/40; A61C 7/10

(57) ABSTRACT

A method provides a palatal expansion management tool. The method includes receiving scan data of a craniofacial structure of a patient, providing the scan data as input to an artificial intelligence (AI) model trained to provide a recommended palatal expansion treatment plan for the patient; receiving, as output from the AI model, the recommended palatal expansion treatment plan; and providing, to a user device, the recommended palatal expansion treatment plan.

### RELATED APPLICATIONS

The present application claims priority to U.S. Provisional Patent Application No. 63/691,955, filed on Sep. 6, 2024, and U.S. Provisional Patent Application No. 63/765,131, filed on Feb. 28, 2025, which are herein incorporated by reference in their entirety.

### TECHNICAL FIELD

The instant specification generally relates to systems and methods for implementing a palatal expansion management tool.

### BACKGROUND

Palatal expansion (PE) is an orthodontic treatment that involves gradually widening the upper jaw in patients to correct various dental and skeletal issues, such as crossbites, overcrowding, and impacted teeth. Palatal expansion treatment is typically prescribed for children or adolescents, as their bones are still developing and more responsive to modification. In some cases, a series of incremental palatal expanders can apply gentle, continuous pressure on the upper molars, causing the two halves of the palate to separate and widen gradually over time. Palatal expansion treatment is often a preliminary step before orthodontic treatment, such as braces or aligners. Palatal expansion treatment plans are adjustable, allowing dental professionals to control the rate and amount of expansion, thereby tailoring the treatment to the patient's specific needs.

### SUMMARY

The below summary is a simplified summary of the disclosure in order to provide a basic understanding of some aspects of the disclosure. This summary is not an extensive overview of the disclosure. It is intended neither to identify key or critical elements of the disclosure, nor delineate any scope of the particular embodiments of the disclosure or any scope of the claims. Its sole purpose is to present some concepts of the disclosure in a simplified form as a prelude to the more detailed description that is presented later.

In a first implementation, a method comprises receiving data of a craniofacial structure of a patient, and identifying one or more parameters corresponding to a palatal expansion treatment plan for the patient. The method further comprises processing the data of the craniofacial structure of the patient and the one or more parameters to generate a visualization of a predicted outcome of the craniofacial structure affected by the palatal expansion treatment plan. The method further comprises providing, for display in a user interface (UI) of a user device, the visualization of the predicted outcome.

A second implementation may further extend the first implementation. In the second implementation, the data of the craniofacial structure of the patient comprises a three-dimensional (3D) dental model.

A third implementation may further extend the first through second implementations. In the third implementation, processing the data may comprise providing the data and the one or more parameters to an artificial intelligence (AI) model trained to provide the predicted outcome of the craniofacial structure caused by the palatal expansion treatment plan. The method may further comprise receiving, as output from the AI model, the predicted outcome of the craniofacial structure caused by the palatal expansion treatment plan, and generating, based on the predicted outcome of the craniofacial structure, the visualization of the predicted outcome of the craniofacial structure.

A fourth implementation may further extend the first through third implementations. In the fourth implementation, the visualization comprises a three-dimensional visualization of the predicted outcome of the craniofacial structure of the patient.

A fifth implementation may further extend the first through fourth implementations. In the fifth implementation, the method further comprises receiving a modification to one of the one or more parameters corresponding to the palatal expansion treatment plan, and providing the modified parameter as additional input to the AI model. The method further comprises receiving, as updated output from the AI model, an updated predicted outcome of the craniofacial structure of the patient. The method further comprises updating the visualization to reflect the updated outcome, and providing, for display in the UI of the user device, the updated visualization.

A sixth implementation may further extend the first through fifth implementations. In the third implementation, the one or more parameters comprise at least one of an amount of expansion, a vertical clearance measurement of an expander, a placement of an attachment on a tooth of a dentition of the patient, a first identification of at least one tooth covered by the expander, a second identification of a corresponding part of a palate of the patient covered by the expander, or a second amount of transverse force applied by the expander.

A seventh implementation may further extend the first through sixth implementations. In the seventh implementation, the predicted outcome includes at least one of an amount of expansion of a palate of the patient, a predicted placement of at least one tooth in a dentition of the patient, or a predicted shape of the palate of the patient.

An eighth implementation may further extend the first through seventh implementations. In the eighth implementation, at least a subset of the one or more parameters is received from the user device.

A ninth implementation may further extend the first through eighth implementations. In the ninth implementation, at least a subset of the one or more parameters corresponds to output received from a second AI model.

A tenth implementation may further extend the first through ninth implementations. In the tenth implementation, the method further comprises generating, based on the processing of the data and the one or more parameters, a three-dimensional dental model of the patient affected by the palatal expansion treatment plan; generating a two-dimensional cross-section of the three-dimensional dental model; and providing, for display in the UI of the user device, the two-dimensional cross-section.

An eleventh implementation may further extend the tenth implementation. In the eleventh implementation, the two-dimensional cross-section and the three-dimensional model are displayed in the UI simultaneously.

A twelfth implementation may further extend the first through eleventh implementations. In the twelfth implementation, generating the two-dimensional cross-section can include identifying a position for the two-dimensional cross-section in a mesial-distal direction of the craniofacial structure of the patient, and generating the two-dimensional cross-section of the three-dimensional model at the identified position.

A thirteenth implementation may further extend the first through twelfth implementations. In the thirteenth implementation, the position is received from the user device.

A fourteenth implementation may further extend the first through thirteenth implementations. In the fourteenth implementation, the user device corresponds to a patient device, a doctor device, or a scanning device.

In a fifteenth implementation, a system comprises a memory and a processing device to execute instructions from the memory to perform the method of the first through fourteenth implementations.

In a sixteenth implementation, a non-transitory computer-readable storage medium comprises instructions that, when executed by a processing device, cause the processing device to perform the method of the first through fourteenth implementations.

In a seventeenth implementation, a method comprises receiving scan data of a craniofacial structure of a patient; generating, based on the scan data, a three-dimensional (3D) dental model comprising an initial breadth of a palate of the patient; generating a palatal expansion treatment plan, wherein the palatal expansion treatment plan comprises a series of breadths of the palate, wherein the series of breadths correspond to a progressive expansion of the palate from the initial breadth toward a target breadth; and providing, to a user device, a 3D visualization of a predicted outcome of the palatal expansion treatment plan.

An eighteenth implementation may further extend the seventeenth implementation. In the eighteenth implementation, the method further comprises providing, to the user device, the palatal expansion treatment plan.

A nineteenth implementation may further extend the seventeenth through eighteenth implementations. In the nineteenth implementation, the palatal expansion treatment plan comprises one or more treatment stages, wherein each of the one or more treatment stages is associated with one or more dental appliances that are usable to implement the palatal expansion treatment plan, and wherein a visual representation of a first dental appliance of the one or more dental appliances is provided for display in a user interface of the user device.

A twentieth implementation may further extend the seventeenth through nineteenth implementations. In the twentieth implementation, the first dental appliance corresponds to a treatment stage of the one or more treatment stages.

A twenty-first implementation may further extend the seventeenth through twentieth implementations. In the twenty-first implementation, at least the treatment stage of the palatal expansion treatment plan is provided for display in a first portion of a user interface (UI) and at least the first dental appliance of the one or more dental appliances is provided for display in a second portion of the UI.

A twenty-second implementation may further extend the seventeenth through twenty-first implementations. In the twenty-second implementation, a visualization of a dentition of the patient and the visualization of the first dental application worn of the dentition are output to a user interface (UI).

A twenty-third implementation may further extend the seventeenth through twenty-second implementation. In the twenty-third implementation, the method further comprises providing the scan data as input to an artificial intelligence (AI) model trained to provide the predicted outcome of the palatal expansion treatment plan; and receiving, as output from the AI model, the predicted outcome of the palatal expansion treatment plan.

A twenty-fourth implementation may further extend the seventeenth through twenty-third implementations. In the twenty-fourth implementation, generating the palatal expansion treatment plan further comprises processing the 3D dental model to obtain the target breadth of the palate; and identifying one or more intermediate breadths in the series of breadths corresponding to the progressive expansion of the palate from the initial breadth toward the target breadth.

A twenty-fifth implementation may further extend the seventeenth through twenty-fourth implementations. In the twenty-fifth implementation, the 3D visualization of the predicted outcome comprises a 3D visualization of the one or more intermediate breadths.

A twenty-sixth implementation may further extend the seventeenth through twenty-fifth implementations. In the twenty-sixth implementation, the 3D visualization of the predicted outcome comprises a 3D visualization of the initial breadth.

A twenty-seventh implementation may further extend the seventeenth through twenty-sixth implementations. In the twenty-seventh implementation, the 3D visualization of the predicted outcome comprises a 3D visualization of the target breadth.

A twenty-eighth implementation may further extend the seventeenth through twenty-seventh implementations. In the twenty-eighth implementation, the 3D visualization of the predicted outcome is provided for display in a user interface (UI) of the user device.

A twenty-ninth implementation may further extend the seventeenth through twenty-eighth implementations. In the twenty-ninth implementation, the UI comprises a UI element associated with an expansion amount corresponding to the target breadth, and the method further comprises receiving, via the UI element, a modification to the expansion amount; generating a modified 3D visualization of the predicted outcome to correspond to the modification to the expansion amount; and providing, to the user device, the modified 3D visualization.

A thirtieth implementation may further extend the seventeenth through twenty-ninth implementations. In the thirtieth implementation, the method further comprises generating, based on the modification to the expansion amount, an updated palatal expansion treatment plan; and providing, to the user device, the updated palatal expansion treatment plan.

A thirty-first implementation may further extend the seventeenth through thirtieth implementations. In the thirty-first implementation, the UI comprises a UI element associated an orientation of the 3D visualization, wherein the UI element enables manipulation of the orientation of the 3D visualization.

A thirty-second implementation may further extend the seventeenth through thirty-first implementations. In the thirty-second implementation, the UI comprises at least one of a first UI element and a second UI element associated with a measurement tool, wherein the first UI element provides one or more measurements of the 3D visualization, and wherein the second UI element enables a user to modify the one or more measurements of the 3D visualization.

A thirty-third implementation may further extend the seventeenth through thirty-second implementations. In the thirty-third implementation, the one or more measurements comprise at least one of an intercuspal jaw width measurement or a buccal overjet measurement.

A thirty-fourth implementation may further extend the seventeenth through thirty-third implementations. In the thirty-fourth implementation, the UI comprises a series of UI elements, wherein each UI element in the series of UI elements corresponds to a palatal expansion treatment plan parameter associated with the palatal expansion treatment plan.

A thirty-fifth implementation may further extend the seventeenth through thirty-fourth implementations. In the thirty-fifth implementation, the palatal expansion treatment plan parameter corresponds to at least one of an amount of expansion, a vertical clearance measurement of an expander, a placement of an attachment on a tooth of a dentition of the patient, a first identification of at least one tooth covered by the expander, a second identification of a corresponding part of a palate of the patient covered by the expander, or a second amount of transverse force applied by the expander.

A thirty-sixth implementation may further extend the seventeenth through thirty-fifth implementations. In the thirty-sixth implementation, the UI comprises a UI element associated with an expansion recommendation corresponding to the patient.

A thirty-seventh implementation may further extend the seventeenth through thirty-sixth implementations. In the thirty-seventh implementation, the UI displays a first set of teeth in the 3D visualization in a first visual representation and a second set of teeth in the 3D visualization in a second visual representation.

A thirty-eighth implementation may further extend the seventeenth through thirty-seventh implementations. In the thirty-eighth implementation, the first set of teeth corresponds to one or more teeth directly affected by the palatal expansion treatment plan, and wherein the first visual representation corresponds to a first shading value.

A thirty-ninth implementation may further extend the seventeenth through thirty-eighth implementations. In the thirty-ninth implementation, the second set of teeth corresponds to one or more teeth indirectly affected by the palatal expansion plan, and wherein the second visual representation corresponds to a second shading value.

A fortieth implementation may further extend the seventeenth through thirty-ninth implementations. In the fortieth implementation, the method further comprising receiving a picture of a face of the patient; generating an image of the face of the patient, wherein the image represents the picture of the face of the patient with the 3D visualization of the predicted outcome integrated into the picture of the face of the patient; and providing, to the user device, the image of the face of the patient.

A forty-first implementation may further extend the seventeenth through fortieth implementations. In the forty-first implementation, the image of the face of the patient and the 3D visualization of the predicted outcome are presented side-by-side in a user interface.

A forty-second implementation may further extend the seventeenth through forty-first implementations. In the forty-second implementation, at least one of the 3D visualization of the predicted outcome or the image of the face of the patient corresponds to one of: (1) a target visualization corresponding to the target breadth, (2) an initial visualization corresponding to the initial breadth, or (3) an intermediate visualization corresponding to a breadth between the initial breadth and the target breadth.

A forty-third implementation may further extend the seventeenth through forty-second implementations. In the forty-third implementation, the 3D visualization is provided for a display in a user interface of at least one of a scanning device, a patient device, or a doctor device.

A forty-fourth implementation may further extend the seventeenth through forty-third implementations. In the forty-fourth implementation, the method further comprises providing one or more dental appliances to implement the palatal expansion treatment plan.

A forty-fifth implementation may further extend the seventeenth through forty-fourth implementations. In the forty-fifth implementation, the method the one or more dental appliances comprises a palatal expander.

A forty-sixth implementation may further extend the seventeenth through forty-fifth implementations. In the forty-sixth implementation, the one or more dental appliances comprises one or more incremental palatal expanders.

A forty-seventh implementation may further extend the seventeenth through forty-sixth implementations. In the forty-seventh implementation, the one or more dental appliances comprise one or more 3D printed incremental palatal expanders.

A forty-eighth implementation may further extend the seventeenth through forty-seventh implementations. In the forty-eighth implementation, the method further comprises providing instructions to fabricate a series of incremental palatal expanders corresponding to the palatal expansion treatment plan.

A forty-ninth implementation may further extend the seventeenth through forty-eighth implementations. In the forty-ninth implementation, the series of incremental palatal expanders comprises at least one intermediate incremental palatal expander corresponding to an intermediate breadth.

A fiftieth implementation may further extend the seventeenth through forty-ninth implementations. In the fiftieth implementation, the series of incremental palatal expanders comprises a final incremental palatal expander corresponding to the target breadth.

A fifty-first implementation may further extend the seventeenth through fiftieth implementations. In the fifty-first implementation, the scan data comprises cone-beam computed tomography data.

A fifty-second implementation may further extend the seventeenth through fifty-first implementations. In the fifty-second implementation, the method further comprises providing the scan data as input to an artificial intelligence (AI) model trained to provide a recommended palatal expansion treatment plan for the patient; and receiving, as output from the AI model, the recommended palatal expansion treatment plan, wherein the palatal expansion treatment plan corresponds to the recommended palatal expansion treatment plan.

In a fifty-third implementation, a system comprises a memory and a processing device to execute instructions from the memory to perform the method of the seventeenth through fifty-second implementations.

In a fifty-fourth implementation, a non-transitory computer-readable storage medium comprises instructions that, when executed by a processing device, cause the processing device to perform the method of the seventeenth through fifty-second implementations

In a fifty-fifth implementation, a method comprises receiving scan data of a craniofacial structure of a patient, and providing the scan data as input to an AI model trained to provide a recommended palatal expansion treatment plan for the patient. The method further comprises receiving, as output from the AI model, the recommended palatal expansion treatment plan, and providing, to the user device, the recommended palatal expansion treatment plan.

A fifty-sixth implementation may further extend the fifty-fifth implementation. In the fifty-sixth implementation, the recommended palatal expansion treatment plan comprises a plurality of treatment stages, each associated with one or more dental appliances that are usable to implement the palatal expansion treatment plan, and wherein a visual representation of a first dental appliance of the one or more dental appliances is provided for display in a user interface of the user device.

A fifty-seventh implementation may further extend the fifth-fifth through fifty-sixth implementations. In the fifty-seventh implementation, the first dental appliance corresponds to a treatment stage of the plurality of treatment stages.

A fifty-eighth implementation may further extend the fifth-fifth through fifty-seventh implementations. In the fifty-eighth implementation, at least the treatment stage of the palatal expansion treatment plan is provided for display in a first portion of a user interface (UI) and at least the first dental appliance of the one or more dental appliances is provided for display in a second portion of the UI.

A fifty-ninth implementation may further extend the fifth-fifth through fifty-eighth implementations. In the fifty-ninth implementation, a visualization of a dentition of the patient and the visualization of the first dental appliance worn over the dentition are output to a user interface (UI).

A sixtieth implementation may further extend the fifth-fifth through fifty-ninth implementations. In the sixtieth implementation, the recommended palatal expansion treatment plan comprises at least one of: an amount of expansion of a palate of the patient or a number of expanders in the recommended palatal expansion treatment plan.

A sixty-first implementation may further extend the fifty-fifth through sixtieth implementations. In the sixty-first implementation, the method further comprises identifying one or more values corresponding to palatal expansion treatment plan parameters, and providing, as additional input to the AI model, the one or more values.

A sixty-second implementation may further extend the fifty-fifth through sixty-first implementations. In the sixty-second implementation, the method further comprises providing, as input to a second AI model, the scan data of the craniofacial structure of the patient and the recommended palatal expansion treatment plan, wherein the second AI model is trained to provide a predicted outcome of the craniofacial structure of the patient; receiving, as output from the second AI model, the predicted outcome of the craniofacial structure of the patient; generating a visualization of the predicted outcome of the craniofacial structure; and providing, for display in a user interface of the user device, the visualization of the predicted outcome.

A sixty-third implementation may further extend the fifty-fifth through sixty-second implementations. In the sixty-third implementation, the method further comprises identifying one or more parameters corresponding to the recommended palatal expansion treatment plan; and providing the one or more parameters as additional input to the second AI model.

A sixty-fourth implementation may further extend the fifty-fifth through sixty-third implementations. In the sixty-fourth implementation, the one or more parameters comprise at least one of an amount of expansion, a vertical clearance measurement of an expander of the recommended palatal expansion treatment plan, a placement of an attachment on a tooth of a dentition of the patient, a first identification of at least one tooth covered by the expander, a second identification of a corresponding part of a palate of the patient covered by the expander, or a second amount of transverse force applied by the expander.

A sixty-fifth implementation may further extend the fifty-fifth through sixty-fourth implementations. In the sixty-fifth implementation, the predicted outcome includes at least one of an amount of expansion of a palate of the patient, a predicted placement of at least one tooth in a dentition of the patient, or a predicted shape of the palate of the patient.

A sixty-sixth implementation may further extend the fifty-fifth through sixty-fifth implementations. In the sixty-sixth implementation, the recommended palatal expansion treatment plan comprises a series of breadths corresponding to a progressive expansion of a palate of the patient from an initial breadth toward a target breadth.

A sixty-seventh implementation may further extend the fifty-fifth through sixty-sixth implementations. In the sixty-seventh implementation, the recommended palatal expansion treatment plan comprises one or more dental appliances to implement the recommended palatal expansion treatment plan.

A sixty-eighth implementation may further extend the fifty-fifth through sixty-seventh implementations. In the sixty-eighth implementation, the one or more dental appliances comprises a palatal expander.

A sixty-ninth implementation may further extend the fifty-fifth through sixty-eighth implementations. In the sixty-ninth implementation, the one or more dental appliances comprises one or more incremental palatal expanders.

A seventieth implementation may further extend the fifty-fifth through sixty-ninth implementations. In the seventieth implementation, the one or more dental appliances comprise one or more 3D printed incremental palatal expanders.

A seventy-first implementation may further extend the fifty-fifth through seventieth implementations. In the seventy-first implementation, the method further comprises providing instructions to fabricate a series of incremental palatal expanders corresponding to the palatal expansion treatment plan.

In a seventy-second implementation, a system comprises a memory and a processing device to execute instructions from the memory to perform the method of the fifty-fifth through seventy-first implementations.

In a seventy-third implementation, a non-transitory computer-readable storage medium comprises instructions that, when executed by a processing device, cause the processing device to perform the method of the fifty-fifth through seventy-first implementations.

In a seventy-fourth implementation, a method comprises receiving scan data of a craniofacial structure of a patient, and identifying one or more parameters corresponding to a palatal expansion treatment plan that comprises a plurality of expanders used to cause movement of one or more parts of the craniofacial structure of the patient. The method further comprises providing the scan data and the one or more parameters to an artificial intelligence (AI) model trained to provide a predicted outcome of the craniofacial structure caused by the palatal expansion treatment plan. The method further comprises receiving, as output from the AI model, the predicted outcome of the craniofacial structure caused by the palatal expansion treatment plan. The method further comprises generating a three-dimensional visualization of the predicted outcome of the craniofacial structure, and providing, for display in a user interface (UI) of a user device, the three-dimensional visualization of the predicted outcome.

A seventy-fifth implementation may further extend the seventy-fourth implementation. In the seventy-fifth implementation, the method further comprises receiving a modification to one of the one or more parameters corresponding to the palatal expansion treatment plan, and providing the modified parameter as additional input to the AI model. The method further comprises receiving, as updated output from the AI model, an updated predicted outcome of the craniofacial structure of the patient. The method further comprises updating the three-dimensional visualization to reflect the updated outcome, and providing, for display in the UI of the user device, the updated three-dimensional visualization.

A seventy-sixth implementation may further extend the seventy-fourth through seventy-fifth implementations. In the seventy-sixth implementation, the one or more parameters comprise at least one of an amount of expansion, a vertical clearance measurement of at least one expander of the plurality of expanders, a placement of an attachment on a tooth of a dentition of the patient, a first identification of at least one tooth covered by the at least one expander of the plurality of expanders, a second identification of a corresponding part of a palate of the patient covered by the at least one expander of the plurality of expanders, or a second amount of transverse force applied by the at least one expander of the plurality of expanders.

A seventy-seventh implementation may further extend the seventy-fouth through seventy-fifth implementations. In the seventy-seventh implementation, the predicted outcome includes at least one of an amount of expansion of a palate of the patient, a predicted placement of at least one tooth in a dentition of the patient, or a predicted shape of the palate of the patient.

A seventy-eighth implementation may further extend the seventy-fourth through seventy-seventh implementations. In the seventy-eighth implementation, at least a subset of the one or more parameters is received from the user device.

A seventy-ninth implementation may further extend the seventy-fourth through seventy-eighth implementations. In the seventy-ninth implementation, at least a subset of the one or more parameters corresponds to output received from a second AI model.

A eightieth implementation may further extend the seventy-fourth through seventy-ninth implementations. In the eightieth implementation, the method further comprises generating, based on the scan data and the one or more parameters, a three-dimensional dental model of the patient affected by the palatal expansion treatment plan; generating a two-dimensional cross-section of the three-dimensional dental model; and providing, for display in the UI of the user device, the two-dimensional cross-section.

An eighty-first implementation may further extend the seventy-fourth through eightieth implementations. In the eighty-first implementation, the two-dimensional cross-section and the three-dimensional model are displayed in the UI simultaneously.

An eighty-second implementation may further extend the seventy-fourth through eighty-first implementations. In the eighty-second implementation, generating the two-dimensional cross-section can include identifying a position for the two-dimensional cross-section in a mesial-distal direction of the craniofacial structure of the patient, and generating the two-dimensional cross-section of the three-dimensional model at the identified position.

An eighty-third implementation may further extend the seventy-fourth through eighty-second implementations. In the eighty-third implementation, the position is received from the user device.

An eighty-fourth implementation may further extend the seventy-fourth through eighty-third implementations. In the eighty-fourth implementation, the user device corresponds to a patient device, a doctor device, or a scanning device.

In an eighty-fifth implementation, a system comprises a memory and a processing device to execute instructions from the memory to perform the method of the seventy-fourth through eighty-fourth implementations.

In an eighty-sixth implementation, a non-transitory computer-readable storage medium comprises instructions that, when executed by a processing device, cause the processing device to perform the method of the seventy-fourth through eighty-fourth implementations.

In an eighty-seventh implementation, a system includes a memory and a processing device to execute instructions from the memory to identify a plurality of reference points within one or more three-dimensional (3D) dental models of a patient. The processing device is further to determine one or more tooth measurements based on the plurality of reference points. The processing device is further to determine, based on the one or more tooth measurements, a recommended palatal expansion amount for a palatal expansion treatment plan for the patient. The processing device is further to provide, to a user device, the recommended palatal expansion amount.

An eighty-eighth implementation may further extend the eighty-seventh implementation. In the eighty-eighth implementation, the one or more 3D dental models comprise a first 3D dental model of an upper jaw and a second 3D dental model of a lower jaw, and the plurality of reference points comprises a first reference point on the first 3D dental model of the upper jaw and a second reference point on the second 3D model of the lower jaw.

An eighty-ninth implementation may further extend the eighty-seventh through eighty-eighth implementations. In the eighty-ninth implementation, the second 3D dental model corresponds to one of an initial stage of a treatment plan for the patient, an intermediary stage of the treatment plan for the patient, or a final stage of the treatment plan for the patient.

A ninetieth implementation may further extend the eighty-seventh through eighty-ninth implementations. In the ninetieth implementation, the treatment plan is an orthodontic alignment treatment plan.

A ninety-first implementation may further extend the eighty-seventh through ninetieth implementations. In the ninety-first implementation, each of the plurality of reference points corresponds to a point on a tooth of the patient.

A ninety-second implementation may further extend the eighty-seventh through ninety-first implementations. In the ninety-second implementation, the processing device is further to receive user input selecting at least a subset of the plurality of reference points.

A ninety-third implementation may further extend the eighty-seventh through ninety-second implementations. In the ninety-third implementation, at least a subset of the plurality of reference points is predetermined.

A ninety-fourth implementation may further extend the eighty-seventh through ninety-third implementations. In the ninety-fourth implementation, the processing device is further to provide the one or more 3D dental models as input to an artificial intelligence (AI) model. The AI model provides a plurality of indications, each indication of the plurality indications representing a location of a reference point within one of the one or more 3D dental models.

A ninety-fifth implementation may further extend the eighty-seventh through ninety-fourth implementations. In the ninety-fifth implementation, the processing device is further to perform image processing on a first 3D dental model of the one or more 3D dental models to determine a location of at least one of the plurality of references points within the first 3D dental model.

A ninety-sixth implementation may further extend the eighty-seventh through ninety-fifth implementations. In the ninety-sixth implementation, a first tooth measurement of the one or more tooth measurements represents a distance between a first reference point on a first tooth and a second reference point on a second tooth of the one or more 3D dental models of the patient.

A ninety-seventh implementation may further extend the eighty-seventh through ninety-sixth implementations. In the ninety-seventh implementation, the first reference point corresponds to a tooth cusp, the first tooth corresponds to an upper-left first molar of an upper jaw, the second reference point corresponds to a second tooth cusp, and the second tooth corresponds to a lower-left first molar of a lower jaw.

A ninety-eighth implementation may further extend the eighty-seventh through ninety-seventh implementations. In the ninety-eighth implementation, the first reference point corresponds to a tooth cusp, the first tooth corresponds to an upper-right first molar of an upper jaw, the second reference point corresponds to a second tooth cusp, and the second tooth corresponds to a lower-right first molar of a lower jaw.

A ninety-ninth implementation may further extend the eighty-seventh through ninety-ninth implementations. In the ninety-ninth implementation, the first reference point represents a first midpoint between a lingual-distal cusp and a lingual-mesial cusp, and the second reference point represents a second midpoint between a buccal-distal cusp and a buccal-mesial cusp.

A hundredth implementation may further extend the eighty-seventh through ninety-ninth implementations. In the hundredth implementation, the one or more 3D dental models comprise one or more first 3D dental models of an upper jaw and a second 3D dental model of a lower jaw, and to determine the recommended palatal expansion amount, the processing device is further to identify a plurality of treatment stages of the palatal expansion treatment plan. Each treatment stage of the plurality of treatment stages corresponds to one of the one or more first 3D dental models of the upper jaw, and each tooth measurement of the one or more tooth measurements corresponds to a treatment stage of the plurality of treatment stages. The processing device is further to identify a minimum tooth measurement of the one or more tooth measurements. The processing device is further to identify a first treatment stage of the plurality of treatment stages, wherein the first treatment stage corresponds to the minimum tooth measurement, and wherein the recommended palatal expansion amount is based on the first treatment stage.

A hundred-first implementation may further extend the eighty-seventh through hundredth implementations. In the hundred-first implementation, to determine the one or more tooth measurements, the processing device is further to determine, for each treatment stage of the plurality of treatment stages, a first distance between a first reference point on a right side of the upper jaw and a second reference point on the right side of the lower jaw, and a second distance between a third reference point on a left side of the upper jaw and a fourth reference point on the left side of the lower jaw.

A hundred-second implementation may further extend the eighty-seventh through hundred-first implementations. In the hundred-second implementation, the first reference point represents a midpoint between a first landmark on a first portion of a first 3D dental model representing the upper jaw and a second landmark on the first portion of the first 3D dental model representing the upper jaw, the second reference point represents a second midpoint between a third landmark on a second portion of a second 3D dental model representing the lower jaw and a fourth landmark on the second portion of the second 3D dental model representing the lower jaw, the first portion corresponds to the second portion, the third reference point represents a third midpoint between a fifth landmark on a third portion of the first 3D dental model representing the upper jaw and a sixth landmark on the third portion of the first 3D dental model representing the upper jaw, the fourth reference point represents a fourth midpoint between a seventh landmark on a fourth portion of the second 3D dental model representing the lower jaw and an eighth landmark on the fourth portion of the second 3D dental model representing the lower jaw, and the third portion corresponds to the fourth portion.

A hundred-third implementation may further extend the eighty-seventh through hundred-second implementations. In the hundred-third implementation, the first portion represents an upper-right first molar, the second portion represents a lower-right first molar, wherein the third portion represents an upper-left first molar, and the fourth portion represents a lower-left first molar.

A hundred-fourth implementation may further extend the eighty-seventh through hundred-third implementations. In the hundred-fourth implementation, the first landmark and the fifth landmark represent a lingual-distal tooth cusp, the second landmark and the sixth landmark represent a lingual-mesial tooth cusp, the third landmark and the seventh landmark represent a buccal-distal tooth cusp, and the fourth landmark and the eighth landmark represent a buccal-mesial tooth cusp.

A hundred-fifth implementation may further extend the eighty-seventh through hundred-fourth implementations. In the hundred-fifth implementation, to determine the one or more tooth measurements, the processing device is further to for each treatment stage of the plurality of treatment stages and for each side of a right side and a left side: generate a first occlusal plane for a first side of the second 3D dental model of the patient, wherein the first occlusal plane extends from a first buccal cusp of a premolar tooth along a second buccal cusp of a molar tooth; generate a second occlusal plane for the second 3D dental model of the patient, wherein the second occlusal plane extends from a tooth crown center of a first-lower molar of the second 3D dental model along a first direction; generate a ray intersecting the first occlusal plane and the second occlusal plane; identify a first midpoint between a lingual-distal cusp and a lingual-mesial cusp of a first-upper molar of the first 3D dental model and a second midpoint between a buccal-distal cusp and a buccal-mesial cusp of the first-lower molar of the second 3D dental model, wherein the first 3D dental model and the second 3D dental model correspond to the particular treatment stage of the plurality of treatment stages; project the first midpoint and the second midpoint to the ray; and calculate a distance between the first midpoint and the second midpoint projected onto the ray, wherein the one or more tooth measurements comprises the distance between the first midpoint and the second midpoint.

A hundred-sixth implementation may further extend the eighty-seventh through hundred-fifth implementations. In the hundred-sixth implementation, to determine the recommended palatal expansion amount, the processing device is further to provide, as input to an artificial intelligence (AI) model, the one or more tooth measurements, wherein the AI model provides the recommend palatal expansion amount.

A hundred-seventh implementation may further extend the eighty-seventh through hundred-sixth implementations. In the hundred-seventh implementation, to provide, to the user device, the recommended palatal expansion amount, the processing device is further to provide, for display in a user interface (UI) of the user device, a representation of the one or more 3D dental models, wherein the representation comprises an indicator of a first reference point of the plurality of reference points; and provide, for display in the UI of the user interface, at least one of the recommended palatal expansion amount, a number of expanders to achieve the recommended palatal expansion amount, or at least one of the one or more tooth measurements.

A hundred-eighth implementation may further extend the eighty-seventh through hundred-seventh implementations. In the hundred-eighth implementation, the processing device is further to provide instructions to fabricate a series of incremental palatal expanders corresponding to the palatal expansion treatment plan.

A hundred-ninth implementation may further extend the eighty-seventh through hundred-eighth implementations. In the hundred-ninth implementation, the user device corresponds to a patient device, a doctor device, or a scanning device.

A hundred-tenth implementation may further extend the eighty-seventh through hundred-ninth implementations. In the hundred-tenth implementation, the one or more 3D dental models are based on scan data of the patient. The scan data is generated using one or more imaging modalities comprising at least one of a cone beam computed tomography (CBCT) scan, a radiograph, a computed tomography (CT) scan, an intraoral scan, a color image, a near-infrared (NIR) image, or an image generated using fluorescence imaging.

In a hundred-eleventh implementation, a method includes identifying a plurality of reference points within one or more three-dimensional (3D) dental models of a patient. The method further comprises determining one or more tooth measurements based on the plurality of reference points. The method further comprises determining, based on the one or more tooth measurements, a recommended palatal expansion amount for a palatal expansion treatment plan for the patient. The method further comprises providing, to a user device, the recommended palatal expansion amount.

A hundred-twelfth implementation may further extend the hundred-eleventh implementation. In the hundred-twelfth implementation, the one or more 3D dental models comprise a first 3D dental model of an upper jaw and a second 3D dental model of a lower jaw, and the plurality of reference points comprises a first reference point on the first 3D dental model of the upper jaw and a second reference point on the second 3D model of the lower jaw.

A hundred-thirteenth implementation may further extend the hundred-eleventh through hundred-twelfth implementations. In the hundred-thirteenth implementation, the second 3D dental model corresponds to one of an initial stage of a treatment plan for the patient, an intermediary stage of the treatment plan for the patient, or a final stage of the treatment plan for the patient.

A hundred-fourteenth implementation may further extend the hundred-eleventh through hundred-thirteenth implementations. In the hundred-fourteenth implementation, the treatment plan is an orthodontic alignment treatment plan.

A hundred-fifteenth implementation may further extend the hundred-eleventh through hundred-fourteenth implementations. In the hundred-fifteenth implementation, each of the plurality of reference points corresponds to a point on a tooth of the patient.

A hundred-sixteenth implementation may further extend the hundred-eleventh through hundred-fifteenth implementations. In the hundred-sixteenth implementation, the method may further include receiving user input selecting at least a subset of the plurality of reference points.

A hundred-seventeenth implementation may further extend the hundred-eleventh through hundred-sixteenth implementations. In the hundred-seventeenth implementation, at least a subset of the plurality of reference points is predetermined.

A hundred-eighteenth implementation may further extend the hundred-eleventh through hundred-seventeenth implementations. In the hundred-eighteenth implementation, the method may further include providing the one or more 3D dental models as input to an artificial intelligence (AI) model, wherein the AI model provides a plurality of indications, each indication of the plurality indications representing a location of a reference point within one of the one or more 3D dental models.

A hundred-nineteenth implementation may further extend the hundred-eleventh through hundred-eighteenth implementations. In the hundred-nineteenth implementation, the method may further include performing image processing on a first 3D dental model of the one or more 3D dental models to determine a location of at least one of the plurality of references points within the first 3D dental model.

A hundred-twentieth implementation may further extend the hundred-eleventh through hundred-nineteenth implementations. In the hundred-twentieth implementation, a first tooth measurement of the one or more tooth measurements represents a distance between a first reference point on a first tooth and a second reference point on a second tooth of the one or more 3D dental models of the patient.

A hundred-twenty-first implementation may further extend the hundred-eleventh through hundred-twentieth implementations. In the hundred-twenty-first implementation, the first reference point corresponds to a tooth cusp, the first tooth corresponds to an upper-left first molar of an upper jaw, the second reference point corresponds to a second tooth cusp, and the second tooth corresponds to a lower-left first molar of a lower jaw.

A hundred-twenty-second implementation may further extend the hundred-eleventh through hundred-twenty-first implementations. In the hundred-twenty-second implementation, the first reference point corresponds to a tooth cusp, the first tooth corresponds to an upper-right first molar of an upper jaw, the second reference point corresponds to a second tooth cusp, and the second tooth corresponds to a lower-right first molar of a lower jaw.

A hundred-twenty-third implementation may further extend the hundred-eleventh through hundred-twenty-second implementations. In the hundred-twenty-third implementation, the first reference point represents a first midpoint between a lingual-distal cusp and a lingual-mesial cusp, and the second reference point represents a second midpoint between a buccal-distal cusp and a buccal-mesial cusp.

A hundred-twenty-fourth implementation may further extend the hundred-eleventh through hundred-twenty-third implementations. In the hundred-twenty-fourth implementation, the one or more 3D dental models comprise one or more first 3D dental models of an upper jaw and a second 3D dental model of a lower jaw. To determine the recommended palatal expansion amount, the method may further include identifying a plurality of treatment stages of the palatal expansion treatment plan, wherein each treatment stage of the plurality of treatment stages corresponds to one of the one or more first 3D dental models of the upper jaw, wherein each tooth measurement of the one or more tooth measurements corresponds to a treatment stage of the plurality of treatment stages; identifying a minimum tooth measurement of the one or more tooth measurements; and identifying a first treatment stage of the plurality of treatment stages, wherein the first treatment stage corresponds to the minimum tooth measurement, and wherein the recommended palatal expansion amount is based on the first treatment stage.

A hundred-twenty-fifth implementation may further extend the hundred-eleventh through hundred-twenty-fourth implementations. In the hundred-twenty-fifth implementation, to determine the one or more tooth measurements, the method may further include determining, for each treatment stage of the plurality of treatment stages, a first distance between a first reference point on a right side of the upper jaw and a second reference point on the right side of the lower jaw, and a second distance between a third reference point on a left side of the upper jaw and a fourth reference point on the left side of the lower jaw.

A hundred-twenty-sixth implementation may further extend the hundred-eleventh through hundred-twenty-fifth implementations. In the hundred-twenty-sixth implementation, the first reference point represents a midpoint between a first landmark on a first portion of a first 3D dental model representing the upper jaw and a second landmark on the first portion of the first 3D dental model representing the upper jaw, the second reference point represents a second midpoint between a third landmark on a second portion of a second 3D dental model representing the lower jaw and a fourth landmark on the second portion of the second 3D dental model representing the lower jaw, the first portion corresponds to the second portion, the third reference point represents a third midpoint between a fifth landmark on a third portion of the first 3D dental model representing the upper jaw and a sixth landmark on the third portion of the first 3D dental model representing the upper jaw, the fourth reference point represents a fourth midpoint between a seventh landmark on a fourth portion of the second 3D dental model representing the lower jaw and an eighth landmark on the fourth portion of the second 3D dental model representing the lower jaw, and the third portion corresponds to the fourth portion.

A hundred-twenty-seventh implementation may further extend the hundred-eleventh through hundred-twenty-sixth implementations. In the hundred-twenty-seventh implementation, the first portion represents an upper-right first molar, wherein the second portion represents a lower-right first molar, wherein the third portion represents an upper-left first molar, and wherein the fourth portion represents a lower-left first molar.

A hundred-twenty-eighth implementation may further extend the hundred-eleventh through hundred-twenty-seventh implementations. In the hundred-twenty-eighth implementation, the first landmark and the fifth landmark represent a lingual-distal tooth cusp, the second landmark and the sixth landmark represent a lingual-mesial tooth cusp, the third landmark and the seventh landmark represent a buccal-distal tooth cusp, and the fourth landmark and the eighth landmark represent a buccal-mesial tooth cusp.

A hundred-twenty-ninth implementation may further extend the hundred-eleventh through hundred-twenty-eighth implementations. In the hundred-twenty-ninth implementation, to determine the one or more tooth measurements, the method may further include for each treatment stage of the plurality of treatment stages and for each side of a right side and a left side: generating a first occlusal plane for a first side of the second 3D dental model of the patient, wherein the first occlusal plane extends from a first buccal cusp of a premolar tooth along a second buccal cusp of a molar tooth; generating a second occlusal plane for the second 3D dental model of the patient, wherein the second occlusal plane extends from a tooth crown center of a first-lower molar of the second 3D dental model along a first direction; generating a ray intersecting the first occlusal plane and the second occlusal plane; identifying a first midpoint between a lingual-distal cusp and a lingual-mesial cusp of a first-upper molar of the first 3D dental model and a second midpoint between a buccal-distal cusp and a buccal-mesial cusp of the first-lower molar of the second 3D dental model, wherein the first 3D dental model and the second 3D dental model correspond to the particular treatment stage; projecting the first midpoint and the second midpoint to the ray; and calculating a distance between the first midpoint and the second midpoint projected onto the ray, wherein the one or more tooth measurements comprises the distance between the first midpoint and the second midpoint.

A hundred-thirtieth implementation may further extend the hundred-eleventh through hundred-twenty-ninth implementations. In the hundred-thirtieth implementation, to determine the recommended palatal expansion amount, the method may further include providing, as input to an artificial intelligence (AI) model, the one or more tooth measurements. The AI model provides the recommended palatal expansion amount.

A hundred-thirty-first implementation may further extend the hundred-eleventh through hundred-thirtieth implementations. In the hundred-thirty-first implementation, to provide, to the user device, the recommended palatal expansion amount, the method my further include providing, for display in a user interface (UI) of the user device, a representation of the one or more 3D dental models, wherein the representation comprises an indicator of a first reference point of the plurality of reference points; and providing, for display in the UI of the user interface, at least one of the recommended palatal expansion amount, a number of expanders to achieve the recommended palatal expansion amount, or at least one of the one or more tooth measurements.

A hundred-thirty-second implementation may further extend the hundred-eleventh through hundred-thirty-first implementations. In the hundred-thirty-second implementation, the method may further include providing instructions to fabricate a series of incremental palatal expanders corresponding to the palatal expansion treatment plan.

A hundred-thirty-third implementation may further extend the hundred-eleventh through hundred-thirty-second implementations. In the hundred-thirty-third implementation, the user device corresponds to a patient device, a doctor device, or a scanning device.

A hundred-thirty-fourth implementation may further extend the hundred-eleventh through hundred-thirty-third implementations. In the hundred-thirty-fourth implementation, the one or more 3D dental models are based on scan data of the patient, the scan data is generated using one or more imaging modalities comprising at least one of a cone beam computed tomography (CBCT) scan, a radiograph, a computed tomography (CT) scan, an intraoral scan, a color image, a near-infrared (NIR) image, or an image generated using fluorescence imaging.

In a hundred-thirty-fifth implementation, a non-transitory computer-readable storage medium comprises instructions that, when executed by a processing device, cause the processing device to perform identify a plurality of reference points within one or more three-dimensional (3D) dental models of a patient. The processing device is further to determine one or more tooth measurements based on the plurality of reference points. The processing device is further to determine, based on the one or more tooth measurements, a recommended palatal expansion amount for a palatal expansion treatment plan for the patient. The processing device is further to provide, to a user device, the recommended palatal expansion amount.

A hundred-thirty-sixth implementation may further extend the hundred-thirty-fifth implementation. In the hundred-thirty-sixth implementation, the one or more 3D dental models comprise a first 3D dental model of an upper jaw and a second 3D dental model of a lower jaw, and the plurality of reference points comprises a first reference point on the first 3D dental model of the upper jaw and a second reference point on the second 3D model of the lower jaw.

A hundred-thirty-seventh implementation may further extend the hundred-thirty-fifth through hundred-thirty-sixth implementations. In the hundred-thirty-sixth implementation, the second 3D dental model corresponds to one of an initial stage of a treatment plan for the patient, an intermediary stage of the treatment plan for the patient, or a final stage of the treatment plan for the patient.

A hundred-thirty-eighth implementation may further extend the hundred-thirty-fifth through eighty-seventh implementations. In the hundred-thirty-eighth implementation, the treatment plan is an orthodontic alignment treatment plan.

A hundred-thirty-ninth implementation may further extend the hundred-thirty-fifth through hundred-thirty-eighth implementations. In the hundred-thirty-ninth implementation, each of the plurality of reference points corresponds to a point on a tooth of the patient.

A hundred-fortieth implementation may further extend the hundred-thirty-fifth through hundred-thirty-ninth implementations. In the hundred-fortieth implementation, the processing device is further to receive user input selecting at least a subset of the plurality of reference points.

A hundred-forty-first implementation may further extend the hundred-thirty-fifth through hundred-fortieth implementations. In the hundred-forty-first implementation, at least a subset of the plurality of reference points is predetermined.

A hundred-forty-second implementation may further extend the hundred-thirty-fifth through hundred-forty-first implementations. In the hundred-forty-second implementation, the processing device is further to provide the one or more 3D dental models as input to an artificial intelligence (AI) model. The AI model provides a plurality of indications, each indication of the plurality indications representing a location of a reference point within one of the one or more 3D dental models.

A hundred-forty-third implementation may further extend the hundred-thirty-fifth through hundred-forty-second implementations. In the hundred-forty-third implementation, the processing device is further to perform image processing on a first 3D dental model of the one or more 3D dental models to determine a location of at least one of the plurality of references points within the first 3D dental model.

A hundred-forty-fourth implementation may further extend the hundred-thirty-fifth through hundred-forty-third implementations. In the hundred-forty-fourth implementation, a first tooth measurement of the one or more tooth measurements represents a distance between a first reference point on a first tooth and a second reference point on a second tooth of the one or more 3D dental models of the patient.

A hundred-forty-fifth implementation may further extend the hundred-thirty-fifth through hundred-forty-fourth implementations. In the hundred-forty-fifth implementation, the first reference point corresponds to a tooth cusp, the first tooth corresponds to an upper-left first molar of an upper jaw, the second reference point corresponds to a second tooth cusp, and the second tooth corresponds to a lower-left first molar of a lower jaw.

A hundred-forty-sixth implementation may further extend the hundred-thirty-fifth through hundred-forty-fifth implementations. In the hundred-forty-sixth implementation, the first reference point corresponds to a tooth cusp, the first tooth corresponds to an upper-right first molar of an upper jaw, the second reference point corresponds to a second tooth cusp, and the second tooth corresponds to a lower-right first molar of a lower jaw.

A hundred-forty-seventh implementation may further extend the hundred-thirty-fifth through hundred-forty-sixth implementations. In the hundred-forty-seventh implementation, the first reference point represents a first midpoint between a lingual-distal cusp and a lingual-mesial cusp, and the second reference point represents a second midpoint between a buccal-distal cusp and a buccal-mesial cusp.

A hundred-forty-eighth implementation may further extend the hundred-thirty-fifth through hundred-forty-seventh implementations. In the hundred-forty-eighth implementation, the one or more 3D dental models comprise one or more first 3D dental models of an upper jaw and a second 3D dental model of a lower jaw, and to determine the recommended palatal expansion amount, the processing device is further to identify a plurality of treatment stages of the palatal expansion treatment plan. Each treatment stage of the plurality of treatment stages corresponds to one of the one or more first 3D dental models of the upper jaw, and each tooth measurement of the one or more tooth measurements corresponds to a treatment stage of the plurality of treatment stages. The processing device is further to identify a minimum tooth measurement of the one or more tooth measurements. The processing device is further to identify a first treatment stage of the plurality of treatment stages, wherein the first treatment stage corresponds to the minimum tooth measurement, and wherein the recommended palatal expansion amount is based on the first treatment stage.

A hundred-forty-ninth implementation may further extend the hundred-thirty-fifth through hundred-forty-eighth implementations. In the hundred-forty-ninth implementation, to determine the one or more tooth measurements, the processing device is further to determine, for each treatment stage of the plurality of treatment stages, a first distance between a first reference point on a right side of the upper jaw and a second reference point on the right side of the lower jaw, and a second distance between a third reference point on a left side of the upper jaw and a fourth reference point on the left side of the lower jaw.

A hundred-fiftieth implementation may further extend the hundred-thirty-fifth through hundred-forty-ninth implementations. In the hundred-fiftieth implementation, the first reference point represents a midpoint between a first landmark on a first portion of a first 3D dental model representing the upper jaw and a second landmark on the first portion of the first 3D dental model representing the upper jaw, the second reference point represents a second midpoint between a third landmark on a second portion of a second 3D dental model representing the lower jaw and a fourth landmark on the second portion of the second 3D dental model representing the lower jaw, the first portion corresponds to the second portion, the third reference point represents a third midpoint between a fifth landmark on a third portion of the first 3D dental model representing the upper jaw and a sixth landmark on the third portion of the first 3D dental model representing the upper jaw, the fourth reference point represents a fourth midpoint between a seventh landmark on a fourth portion of the second 3D dental model representing the lower jaw and an eighth landmark on the fourth portion of the second 3D dental model representing the lower jaw, and the third portion corresponds to the fourth portion.

A hundred-fifty-first implementation may further extend the hundred-thirty-fifth through hundred-fiftieth implementations. In the hundred-fifty-first implementation, the first portion represents an upper-right first molar, the second portion represents a lower-right first molar, wherein the third portion represents an upper-left first molar, and the fourth portion represents a lower-left first molar.

A hundred-fifty-second implementation may further extend the hundred-thirty-fifth through hundred-fifty-first implementations. In the hundred-fifty-second implementation, the first landmark and the fifth landmark represent a lingual-distal tooth cusp, the second landmark and the sixth landmark represent a lingual-mesial tooth cusp, the third landmark and the seventh landmark represent a buccal-distal tooth cusp, and the fourth landmark and the eighth landmark represent a buccal-mesial tooth cusp.

A hundred-fifty-third implementation may further extend the hundred-thirty-fifth through hundred-fifty-second implementations. In the hundred-fifty-third implementation, to determine the one or more tooth measurements, the processing device is further to for each treatment stage of the plurality of treatment stages and for each side of a right side and a left side: generate a first occlusal plane for a first side of the second 3D dental model of the patient, wherein the first occlusal plane extends from a first buccal cusp of a premolar tooth along a second buccal cusp of a molar tooth; generate a second occlusal plane for the second 3D dental model of the patient, wherein the second occlusal plane extends from a tooth crown center of a first-lower molar of the second 3D dental model along a first direction; generate a ray intersecting the first occlusal plane and the second occlusal plane; identify a first midpoint between a lingual-distal cusp and a lingual-mesial cusp of a first-upper molar of the first 3D dental model and a second midpoint between a buccal-distal cusp and a buccal-mesial cusp of the first-lower molar of the second 3D dental model, wherein the first 3D dental model and the second 3D dental model correspond to the particular treatment stage of the plurality of treatment stages; project the first midpoint and the second midpoint to the ray; and calculate a distance between the first midpoint and the second midpoint projected onto the ray, wherein the one or more tooth measurements comprises the distance between the first midpoint and the second midpoint.

A hundred-fifty-fourth implementation may further extend the hundred-thirty-fifth through hundred-fifty-third implementations. In the hundred-fifty-fourth implementation, to determine the recommended palatal expansion amount, the processing device is further to provide, as input to an artificial intelligence (AI) model, the one or more tooth measurements, wherein the AI model provides the recommend palatal expansion amount.

A hundred-fifty-fifth implementation may further extend the hundred-thirty-fifth through hundred-fifty-fourth implementations. In the hundred-fifty-fifth implementation, to provide, to the user device, the recommended palatal expansion amount, the processing device is further to provide, for display in a user interface (UI) of the user device, a representation of the one or more 3D dental models, wherein the representation comprises an indicator of a first reference point of the plurality of reference points; and provide, for display in the UI of the user interface, at least one of the recommended palatal expansion amount, a number of expanders to achieve the recommended palatal expansion amount, or at least one of the one or more tooth measurements.

A hundred-fifty-sixth implementation may further extend the hundred-thirty-fifth through hundred-fifty-fifth implementations. In the hundred-fifty-sixth implementation, the processing device is further to provide instructions to fabricate a series of incremental palatal expanders corresponding to the palatal expansion treatment plan.

A hundred-fifty-seventh implementation may further extend the hundred-thirty-fifth through hundred-fifty-sixth implementations. In the hundred-fifty-seventh implementation, the user device corresponds to a patient device, a doctor device, or a scanning device.

A hundred-fifty-eighth implementation may further extend the hundred-thirty-fifth through hundred-fifty-seventh implementations. In the hundred-fifty-eighth implementation, the one or more 3D dental models are based on scan data of the patient. The scan data is generated using one or more imaging modalities comprising at least one of a cone beam computed tomography (CBCT) scan, a radiograph, a computed tomography (CT) scan, an intraoral scan, a color image, a near-infrared (NIR) image, or an image generated using fluorescence imaging.

In a hundred-fifty-ninth implementation, a method includes identifying one or more three-dimensional (3D) dental models of a patient. The one or more 3D dental models correspond to a palatal expansion treatment plan for the patient. The method may further include determining a two-dimensional (2D) cross-section of each the one or more 3D dental models that intersects a first portion of the each of the one or more 3D dental models at a corresponding first location. The method may further include providing, for display on a user interface (UI) of a user device, the 2D cross-section of each of the one or more 3D dental models. The method may further include determining one or more palatal expansion measurements at the 2D cross-section for an amount of palatal expansion associated with the palatal expansion treatment plan at a stage of treatment. The method may further include providing, for display on the UI of the user device, the one or more palatal expansion measurements.

A hundred-sixtieth implementation may further extend the hundred-fifty-ninth implementation. In the hundred-sixtieth implementation, the method may further include responsive to receiving a user input, generating a second 2D cross-section to intersect each of the one or more 3D dental models at a second location. The method may further include providing, for display on the UI of the user device, the second 2D cross-section of each of the one or more 3D dental models at the second location. The method may further include determining one or more second palatal expansion measurements at the second 2D cross-section for a second amount of palatal expansion associated with the palatal expansion treatment plan at a second stage of treatment. The method may further include providing, for display on the UI of the user device, the one or more second palatal expansion measurements.

A hundred-sixty-first implementation may further extend the hundred-fifty-ninth through hundred-sixtieth implementations. In the hundred-sixty-first implementation, a first palatal expansion measurement is associated with a first reference point of a plurality of reference points, and the first reference point corresponds to the first portion. The method may further include determining a first location the first reference point on the 2D cross-section; and providing, for display on the UI of the user device, a first indicator representing the first location of the first reference point on the 2D cross-section.

A hundred-sixty-second implementation may further extend the hundred-fifty-ninth through hundred-sixty-first implementations. In the hundred-sixty-second implementation, the method may further include providing, for display on the UI of the user device, a second indicator representing the first location of the first reference point on the 3D dental model.

A hundred-sixty-third implementation may further extend the hundred-fifty-ninth through hundred-sixty-second implementations. In the hundred-sixty-third implementation, the method may further include receiving user input selecting at least a subset of the plurality of reference points.

A hundred-sixty-fourth implementation may further extend the hundred-fifty-ninth through hundred-sixty-third implementations. In the hundred-sixty-fourth implementation, wherein at least a subset of the plurality of reference points is predetermined.

A hundred-sixty-fifth implementation may further extend the hundred-fifty-ninth through hundred-sixty-fourth implementations. In the hundred-sixty-fifth implementation, the method may further include providing the one or more 3D dental models as input to an artificial intelligence (AI) model, wherein the AI model provides a plurality of indications, each indication of the plurality indications representing a location of a reference point within one of the one or more 3D dental models.

A hundred-sixty-sixth implementation may further extend the hundred-fifty-ninth through hundred-sixty-fifth implementations. In the hundred-sixty-sixth implementation, the method may further include performing image processing on a first 3D dental model of the one or more 3D dental models to determine a location of at least one of the plurality of references points within the first 3D dental model.

A hundred-sixty-seventh implementation may further extend the hundred-fifty-ninth through hundred-sixty-sixth implementations. In the hundred-sixty-seventh implementation, the method may further include outputting an occlusal-view photograph at a first region of the UI. The method may further include outputting the 2D cross-section at a second region of the UI, wherein the occlusal-view photograph corresponds to the 2D cross-section.

A hundred-sixty-eighth implementation may further extend the hundred-fifty-ninth through hundred-sixty-seventh implementations. In the hundred-sixty-eighth implementation, the occlusal-view photograph comprises at least the first portion and an indication of a location of the 2D cross-section.

A hundred-sixty-ninth implementation may further extend the hundred-fifty-ninth through hundred-sixty-eighth implementations. In the hundred-sixty-ninth implementation, the 2D cross-section represents a side-view of the first portion of the 3D dental model.

A hundred-seventieth implementation may further extend the hundred-fifty-ninth through hundred-sixty-ninth implementations. In the hundred-seventieth implementation, the method may further include providing a ruler tool in the UI of the user device, wherein the ruler tool provides a visualization of at least one of the one or more palatal expansion measurements.

A hundred-seventy-first implementation may further extend the hundred-fifty-ninth through hundred-seventieth implementations. In the hundred-seventy-first implementation, the method may further include providing, for display on the UI of the user device, the one or more 3D dental models and an indication of the corresponding first location on the one or more 3D dental models.

A hundred-seventy-second implementation may further extend the hundred-fifty-ninth through hundred-seventy-first implementations. In the hundred-seventy-second implementation, the first portion corresponds to a 3D object of the one or more 3D dental models, the corresponding first location corresponds to a first reference point of the 3D object, and the first reference point corresponds to at least one of a surface point or an internal point of the 3D object.

A hundred-seventy-third implementation may further extend the hundred-fifty-ninth through hundred-seventy-second implementations. In the hundred-seventy-third implementation, the first portion of the 3D dental model corresponds to a tooth of the patient.

A hundred-seventy-fourth implementation may further extend the hundred-fifty-ninth through hundred-seventy-third implementations. In the hundred-seventy-fourth implementation, the 2D cross-section of the one or more 3D dental models illustrates both sides of an upper jaw of the patient and both sides of a lower jaw of the patient.

A hundred-seventy-fifth implementation may further extend the hundred-fifty-ninth through hundred-seventy-fourth implementations. In the hundred-seventy-fifth implementation, the 2D cross-section of the one or more 3D dental models is displayed in color.

A hundred-seventy-sixth implementation may further extend the hundred-fifty-ninth through hundred-seventy-fifth implementations. In the hundred-seventy-sixth implementation, the 2D cross-section of the one or more 3D dental models is displayed in black and white.

A hundred-seventy-seventh implementation may further extend the hundred-fifty-ninth through hundred-seventy-sixth implementations. In the hundred-seventy-seventh implementation, the user device corresponds to a patient device, a doctor device, or a scanning device.

A hundred seventy-eighth implementation may further extend the hundred-fifty-ninth through hundred-seventy-seventh implementations. In the hundred-seventy-eighth implementation, at least one of the one or more 3D dental models are based on scan data of the patient. The scan data is generated using one or more imaging modalities comprising at least one of a cone beam computed tomography (CBCT) scan, a radiograph, a computed tomography (CT) scan, an intraoral scan, a color image, a near-infrared (NIR) image, or an image generated using fluorescence imaging.

In a hundred-seventy-ninth implementation, a system includes a memory and a processing device to execute instructions from the memory to identify one or more three-dimensional (3D) dental models of a patient, wherein the one or more 3D dental models correspond to a palatal expansion treatment plan for the patient. The processing device is further to determine a two-dimensional (2D) cross-section of each the one or more 3D dental models that intersects a first portion of the each of the one or more 3D dental models at a corresponding first location. The processing device is further to provide, for display on a user interface (UI) of a user device, the 2D cross-section of each of the one or more 3D dental models. The processing device is further to determine one or more palatal expansion measurements at the 2D cross-section for an amount of palatal expansion associated with the palatal expansion treatment plan at a stage of treatment. The processing device is further to provide, for display on the UI of the user device, the one or more palatal expansion measurements.

A hundred-eightieth implementation may further extend the hundred-seventy-ninth implementation. In the hundred-eightieth implementation, responsive to receiving a user input, the processing is further to generate a second 2D cross-section to intersect each of the one or more 3D dental models at a second location. The processing is further to provide, for display on the UI of the user device, the second 2D cross-section of each of the one or more 3D dental models at the second location. The processing device is further to determine one or more second palatal expansion measurements at the second 2D cross section for a second amount of palatal expansion associated with the palatal expansion treatment plan at a second stage of treatment. The processing device is further to provide, for display on the UI of the user device, the one or more second palatal expansion measurements.

A hundred-eighty-first implementation may further extend the hundred-seventy-ninth through hundred-eightieth implementations. In the hundred-eighty-first implementation, a first palatal expansion measurement is associated with a first reference point of a plurality of reference points. The first reference point corresponds to the first portion. The processing device is further to determine a first location the first reference point on the 2D cross-section; and provide, for display on the UI of the user device, a first indicator representing the first location of the first reference point on the 2D cross-section.

A hundred-eighty-second implementation may further extend the hundred-seventy-ninth through hundred-eighty-first implementations. In the hundred-eighty-second implementation, the processing device is further to provide, for display on the UI of the user device, a second indicator representing the first location of the first reference point on the 3D dental model.

A hundred-eighty-third implementation may further extend the hundred-seventy-ninth through hundred-eighty-second implementations. In the hundred-eighty-third implementation, the processing device is further to receive user input selecting at least a subset of the plurality of reference points.

A hundred-eighty-fourth implementation may further extend the hundred-seventy-ninth through hundred-eighty-third implementations. In the hundred-eighty-fourth implementation, at least a subset of the plurality of reference points is predetermined.

A hundred-eighty-fifth implementation may further extend the hundred-seventy-ninth through hundred-eighty-fourth implementations. In the hundred-eighty-fifth implementation, the processing device is further to provide the one or more 3D dental models as input to an artificial intelligence (AI) model, wherein the AI model provides a plurality of indications, each indication of the plurality indications representing a location of a reference point within one of the one or more 3D dental models.

A hundred-eighty-sixth implementation may further extend the hundred-seventy-ninth through hundred-eighty-fifth implementations. In the hundred-eighty-sixth implementation, the processing device is further to perform image processing on a first 3D dental model of the one or more 3D dental models to determine a location of at least one of the plurality of references points within the first 3D dental model.

A hundred-eighty-seventh implementation may further extend the hundred-seventy-ninth through hundred-eighty-sixth implementations. In the hundred-eighty-seventh implementation, the processing device is further to output an occlusal-view photograph at a first region of the UI, and output the 2D cross-section at a second region of the UI, wherein the occlusal-view photograph corresponds to the 2D cross-section.

A hundred-eighty-eighth implementation may further extend the hundred-seventy-ninth through hundred-eighty-seventh implementations. In the hundred-eighty-eighth implementation, the occlusal-view photograph comprises at least the first portion and an indication of a location of the 2D cross-section.

A hundred-eighty-ninth implementation may further extend the hundred-seventy-ninth through hundred-eighty-eighth implementations. In the hundred-eighty-ninth implementation, the 2D cross-section represent a side-view of the first portion of the 3D dental model.

A hundred-ninetieth implementation may further extend the hundred-seventy-ninth through hundred-eighty-ninth implementations. In the hundred-ninetieth implementation, the processing device is further to provide a ruler tool in the UI of the user device, wherein the ruler tool provides a visualization of at least one of the one or more palatal expansion measurements.

A hundred-ninety-first implementation may further extend the hundred-seventy-ninth through hundred-ninetieth implementations. In the hundred-ninety-first implementation, the processing device is further to provide, for display on the UI of the user device, the one or more 3D dental models and an indication of the corresponding first location on the one or more 3D dental models.

A hundred-ninety-second implementation may further extend the hundred-seventy-ninth through hundred-ninety-first implementations. In the hundred-ninety-second implementation, the first portion corresponds to a 3D object of the one or more 3D dental models, the corresponding first location corresponds to a first reference point of the 3D object, and the first reference point corresponds to at least one of a surface point or an internal point of the 3D object.

A hundred-ninety-third implementation may further extend the hundred-seventy-ninth through hundred-ninety-second implementations. In the hundred-ninety-third implementation, the first portion of the 3D dental model corresponds to a tooth of the patient.

A hundred-ninety-fourth implementation may further extend the hundred-seventy-ninth through hundred-ninety-third implementations. In the hundred-ninety-fourth implementation, the 2D cross-section of the one or more 3D dental models illustrates both sides of an upper jaw of the patient and both sides of a lower jaw of the patient.

A hundred-ninety-fifth implementation may further extend the hundred-seventy-ninth through hundred-ninety-fourth implementations. In the hundred-ninety-fifth implementation, the 2D cross-section of the one or more 3D dental models is displayed in color.

A hundred-ninety-sixth implementation may further extend the hundred-seventy-ninth through hundred-ninety-fifth implementations. In the hundred-ninety-sixth implementation, the 2D cross-section of the one or more 3D dental models is displayed in black and white.

A hundred-ninety-seventh implementation may further extend the hundred-seventy-ninth through hundred-ninety-sixth implementations. In the hundred-ninety-seventh implementation, the user device corresponds to a patient device, a doctor device, or a scanning device.

A hundred-ninety-eightieth implementation may further extend the hundred-seventy-ninth through hundred-ninety-seventh implementations. In the hundred-ninety-eightieth implementation, at least one of the one or more 3D dental models are based on scan data of the patient, the scan data is generated using one or more imaging modalities comprising at least one of a cone beam computed tomography (CBCT) scan, a radiograph, a computed tomography (CT) scan, an intraoral scan, a color image, a near-infrared (NIR) image, or an image generated using fluorescence imaging.

In a hundred-ninety-ninth implementation, a non-transitory computer-readable storage medium comprises instructions that, when executed by a processing device, cause the processing device to identify one or more three-dimensional (3D) dental models of a patient, wherein the one or more 3D dental models correspond to a palatal expansion treatment plan for the patient. The processing device is further to determine a two-dimensional (2D) cross-section of each the one or more 3D dental models that intersects a first portion of the each of the one or more 3D dental models at a corresponding first location. The processing device is further to provide, for display on a user interface (UI) of a user device, the 2D cross-section of each of the one or more 3D dental models. The processing device is further to determine one or more palatal expansion measurements at the 2D cross-section for an amount of palatal expansion associated with the palatal expansion treatment plan at a stage of treatment. The processing device is further to provide, for display on the UI of the user device, the one or more palatal expansion measurements.

A two-hundredth implementation may further extend the hundred-ninety-ninth implementation. In the two-hundredth implementation, responsive to receiving a user input, the processing is further to generate a second 2D cross-section to intersect each of the one or more 3D dental models at a second location. The processing is further to provide, for display on the UI of the user device, the second 2D cross-section of each of the one or more 3D dental models at the second location. The processing device is further to determine one or more second palatal expansion measurements at the second 2D cross section for a second amount of palatal expansion associated with the palatal expansion treatment plan at a second stage of treatment. The processing device is further to provide, for display on the UI of the user device, the one or more second palatal expansion measurements.

A two-hundred-first implementation may further extend the hundred-ninety-ninth through two-hundredth implementations. In the two-hundred-first implementation, a first palatal expansion measurement is associated with a first reference point of a plurality of reference points. The first reference point corresponds to the first portion. The processing device is further to determine a first location the first reference point on the 2D cross-section; and provide, for display on the UI of the user device, a first indicator representing the first location of the first reference point on the 2D cross-section.

A two-hundred-second implementation may further extend the hundred-ninety-ninth through two-hundred-first implementations. In the two-hundred-second implementation, the processing device is further to provide, for display on the UI of the user device, a second indicator representing the first location of the first reference point on the 3D dental model.

A two-hundred-third implementation may further extend the hundred-ninety-ninth through two-hundred-second implementations. In the two-hundred-third implementation, the processing device is further to receive user input selecting at least a subset of the plurality of reference points.

A two-hundred-fourth implementation may further extend the hundred-ninety-ninth through two-hundred-third implementations. In the two-hundred-fourth implementation, at least a subset of the plurality of reference points is predetermined.

A two-hundred-fifth implementation may further extend the hundred-ninety-ninth through two-hundred-fourth implementations. In the two-hundred-fifth implementation, the processing device is further to provide the one or more 3D dental models as input to an artificial intelligence (AI) model, wherein the AI model provides a plurality of indications, each indication of the plurality indications representing a location of a reference point within one of the one or more 3D dental models.

A two-hundred-sixth implementation may further extend the hundred-ninety-ninth through two-hundred-fifth implementations. In the two-hundred-sixth implementation, the processing device is further to perform image processing on a first 3D dental model of the one or more 3D dental models to determine a location of at least one of the plurality of references points within the first 3D dental model.

A two-hundred-seventh implementation may further extend the hundred-ninety-ninth through two-hundred-sixth implementations. In the two-hundred-seventh implementation, the processing device is further to output an occlusal-view photograph at a first region of the UI, and output the 2D cross-section at a second region of the UI, wherein the occlusal-view photograph corresponds to the 2D cross-section.

A two-hundred-eighth implementation may further extend the hundred-ninety-ninth through two-hundred-seventh implementations. In the two-hundred-eighth implementation, the occlusal-view photograph comprises at least the first portion and an indication of a location of the 2D cross-section.

A two-hundred-ninth implementation may further extend the hundred-ninety-ninth through two-hundred-eighth implementations. In the two-hundred-ninth implementation, the 2D cross-section represent a side-view of the first portion of the 3D dental model.

A two-hundred-tenth implementation may further extend the hundred-ninety-ninth through two-hundred-ninth implementations. In the two-hundred-tenth implementation, the processing device is further to provide a ruler tool in the UI of the user device, wherein the ruler tool provides a visualization of at least one of the one or more palatal expansion measurements.

A two-hundred-eleventh implementation may further extend the hundred-ninety-ninth through two-hundred-tenth implementations. In the two-hundred-eleventh implementation, the processing device is further to provide, for display on the UI of the user device, the one or more 3D dental models and an indication of the corresponding first location on the one or more 3D dental models.

A two-hundred-twelfth implementation may further extend the hundred-ninety-ninth through two-hundred-eleventh implementations. In the two-hundred-twelfth implementation, the first portion corresponds to a 3D object of the one or more 3D dental models, the corresponding first location corresponds to a first reference point of the 3D object, and the first reference point corresponds to at least one of a surface point or an internal point of the 3D object.

A two-hundred-thirteenth implementation may further extend the hundred-ninety-ninth through two-hundred-twelfth implementations. In the two-hundred-thirteenth implementation, the first portion of the 3D dental model corresponds to a tooth of the patient.

A two-hundred-fourteenth implementation may further extend the hundred-ninety-ninth through two-hundred-thirteenth implementations. In the two-hundred-fourteenth implementation, the 2D cross-section of the one or more 3D dental models illustrates both sides of an upper jaw of the patient and both sides of a lower jaw of the patient.

A two-hundred-fifteenth implementation may further extend the hundred-ninety-ninth through two-hundred-fourteenth implementations. In the two-hundred-fifteenth implementation, the 2D cross-section of the one or more 3D dental models is displayed in color.

A two-hundred-sixteenth implementation may further extend the hundred-ninety-ninth through two-hundred-fifteenth implementations. In the two-hundred-sixteenth implementation, the 2D cross-section of the one or more 3D dental models is displayed in black and white.

A two-hundred-seventeenth implementation may further extend the hundred-ninety-ninth through two-hundred-sixteenth implementations. In the two-hundred-seventeenth implementation, the user device corresponds to a patient device, a doctor device, or a scanning device.

A two-hundred-eighteenth implementation may further extend the hundred-ninety-ninth through two-hundred-seventeenth implementations. In the two-hundred-eighteenth implementation, at least one of the one or more 3D dental models are based on scan data of the patient, the scan data is generated using one or more imaging modalities comprising at least one of a cone beam computed tomography (CBCT) scan, a radiograph, a computed tomography (CT) scan, an intraoral scan, a color image, a near-infrared (NIR) image, or an image generated using fluorescence imaging.

### BRIEF DESCRIPTION OF THE DRAWINGS

Aspects and embodiments of the present disclosure will be understood more fully from the detailed description given below and from the accompanying drawings of various aspects and embodiments of the disclosure, which, however, should not be taken to limit the disclosure to the specific aspects or embodiments, but are for explanation and understanding only.

FIG. 1 shows a block diagram of an example system for implementing a palatal expansion previewer and treatment management tool, in accordance with some embodiments of the present disclosure.

FIG. 2 depicts an example user interface of a palatal expansion previewer and treatment management tool, in accordance with some embodiments of the present disclosure.

FIG. 3A depicts a portion of an example user interface of a palatal expansion previewer and treatment management tool displaying a two dimensional view, in accordance with some embodiments of the present disclosure.

FIG. 3B depicts a portion of an example user interface of a palatal expansion previewer and treatment management tool displaying a two dimensional view and a three dimensional view, in accordance with some embodiments of the present disclosure.

FIG. 4A illustrates a flow diagram of an example method for providing a visualization of a predicted outcome of a palatal expansion treatment plan, in accordance with some embodiments of the present disclosure.

FIG. 4B illustrates a flow diagram of an example method for providing a recommended palatal expansion treatment plan, in accordance with some embodiments of the present disclosure.

FIG. 4C illustrates a flow diagram of an example method for presenting a predicted outcome in a UI, in accordance with some embodiments of the present disclosure.

FIG. 4D illustrates a flow diagram of an example method for providing a three-dimensional visualization of a predicted outcome of a palatal expansion treatment plan, in accordance with some embodiments of the present disclosure.

FIG. 5 illustrates workflows for training and implementing one or more artificial intelligence models to provide a palatal expansion treatment plan recommendation and/or a palatal expansion outcome, in accordance with some embodiments of the present disclosure.

FIG. 6 illustrates a block diagram of an example computing device, in accordance with some embodiments of the present disclosure.

FIG. 7 illustrates a portion of an example user interface of a palatal expansion previewer and treatment management tool displaying an “auto setup” feature, in accordance with some embodiments of the present disclosure.

FIG. 8 illustrates a portion of an example user interface of a palatal expansion previewer and treatment management tool displaying reference points, in accordance with some embodiments of the present disclosure.

FIG. 9 illustrates a portion of an example user interface of a palatal expansion previewer and treatment management tool displaying occlusal view images of a patient's teeth along with two-dimensional cross-sectional visualizations of teeth, in accordance with some embodiments of the present disclosure.

FIG. 10 illustrates a portion of an example user interface of a palatal expansion previewer and treatment management tool displaying the visualization of a cross-sectional plane with optional indicators of reference points, in accordance with some embodiments of the present disclosure.

FIG. 11 illustrates a portion of an example user interface of a palatal expansion previewer and treatment management tool a ruler tool, in accordance with some embodiments of the present disclosure.

FIG. 12 illustrates a flow diagram of an example method for determining an upper arch expansion amount based on a lower arch final position, in accordance with some embodiments of the present disclosure.

FIG. 13 illustrates a flow diagram of an example method for visualizing a cross-sectional plane and reference points for PE treatment, in accordance with some embodiments of the present disclosure.

### DETAILED DESCRIPTION

Aspects of the present disclosure are directed to providing a treatment planning and visualization tool for palatal expansion (PE). The PE treatment can use a palatal expander or a series of palatal expanders that gradually widen the palate in a patient's upper jaw, to create space in a patient's mouth. For example, palatal expansion can be prescribed to create space in a child's mouth, to allow for adult teeth to come in. As another example, since the upper arch should traditionally be wider than the lower arch, palatal expansion can be prescribed when the lower arch is wider than the upper arch. An expander can sit on the back top teeth, and may be supported via attachments on the teeth in embodiments. An attachment can be bonded to the back teeth (e.g., the molars), and the expander can hook into the attachment for retention. The expander can apply transverse force to the back teeth (e.g., the molars and/or premolars of the patient), which can result in a transverse expansion of the palate. The expander applies pressure to the upper jawbone, causing the upper arch palatal suture to gradually widen as the bones in the upper jaw move apart, thus creating more space in the upper jaw. The expander can apply constant force while it is inserted in a mouth. An expander can be worn continuously, including while the patient is eating or drinking. In some cases, the expanders can be designed to be replaced every day, or every few days, for example. In some embodiments, the expanders can be 3D printed, e.g., can be made of 3D printed nylon.

Orthodontic treatment planning for upper arch expansion is traditionally a highly manual and subjective process, during which clinicians rely on their manual measurements to determine the appropriate amount of expansion and to visual treatment outcomes. Conventional treatment planning often involves interpretation of complex 3D models or static 2D images to manually identify landmarks and estimate expansion targets. This can be time consuming and prone to inconsistencies and human error, leading to variability in treatment quality and efficiency.

The PE treatment plan can be adjustable and customized for each patient. For example, a dental professional can prescribe a specific expansion amount, and each expander in the series of expanders can be designed to widen the jaw a fraction of the prescribed expansion amount. However, it can be difficult for dental professionals to predict and visualize the final result of the prescribed expansion amount, and how the final result of the upper jaw expansion will interact with the tooth positioning of the lower jaw. Traditional PE treatment can require regular monitoring and frequent treatment adjustments to ensure the expansion amount is in line with the expansion goals.

Aspects of the present discourse can address the above-noted and other challenges by providing a visualization and treatment planning software (sometimes referred to as “the software”) for a PE treatment, enabling a dental professional (e.g., a doctor, a dentist, a hygienist, a clinician, and/or a technician) to plan the palatal expansion digitally and/or track palatal expansion. The software can provide a visualization of the likely outcome of the palatal expansion. The likely outcome can include predicted placement of the upper jaw in relation to the lower jaw and optionally predicted tooth movement. The predicted outcome can be used in conjunction with other orthodontic treatments (e.g., an aligner orthodontic treatment plan). In some embodiments, the software can provide a suggested expansion amount for a particular patient.

When used as a planning tool, the software can enable the dental professional to modify certain parameters of the PE treatment. The software can include a set of controls to enable the dental professional to modify the parameters. As the dental professional modifies the parameters using the controls, the software can automatically update the likely outcome of the PE treatment. The parameters can include, for example, the amount of expansion, the vertical clearance (e.g., the “width” or “thickness” of the expander), the attachment placement, which teeth are covered by the expander, which part of the palate is covered by the expander, the amount of transverse force applied by the expander, etc. In some embodiments, the software can provide a recommended expansion amount for a particular patient.

In some embodiments, the software can implement an artificial intelligence (AI) model that is trained to provide a recommended PE treatment. In some embodiments, the AI model can be trained on a set of training data that includes previous PE treatment plans and their outcomes. The AI model can receive the scan data of a particular patient as input, and can provide a recommended PE treatment plan as output. The scan data may include a set of intraoral scans of the patient, a 3D model of an upper and/or lower dentition of the patient (e.g., upper and/or lower dental arch), one or more projections of a 3D model of the upper and/or lower dentition onto one or more planes, and so on. In some embodiments, the software can accommodate preferences of the dental professional, e.g., by providing PE treatment plans previously prescribed by the dental professional and/or specific preferences of the dental professional as additional input to the AI model. In some embodiments, the software can identify patterns in the dental professional's previous PE treatment plans, which can be used to personalize the recommend PE treatment plans for the particular dental professional. The recommended PE treatment plan provided by the AI model can include a recommended expansion amount, a recommended number of expanders, a frequency of expander replacement (or wear-time for each expander), and/or other PE treatment plan parameters.

In some embodiments, the software can provide a recommended expansion amount based on a position of patient's lower jaw. The position of the patient's lower jaw can correspond to the current position of the patient's lower jaw, or to an expected position of the patient's lower jaw following a treatment plan (e.g., an orthodontic treatment plan). The software can determine a recommended expansion amount using anatomical landmarks such as molar cusps. Since the upper arch should be wider than the lower arch, in general, the buccal cusp of the upper first molar should be buccal to the buccal cusp of the lower first molar. During expansion of the upper arch sutural split, the desired final position can be when the lingual cusps of the upper first molar are in the plane of buccal cusps of the lower first molar. Such a final position results in the upper and lower teeth remaining in contact (e.g., occlusion), and may also allow the upper molars to provide pressure on the lower molars to expand the lower jaw. Overexpanding the upper arch past the point of occlusion can cause instability and put the patient in a reverse crossbite. However, not expanding the upper arch enough may not achieve the treatment goals, such as creating space for erupting dentition to resolve crowding. Thus, the recommended expansion amount reflects a final position that is neither over-expanded nor under-expanded, e.g., to provide functional occlusion and treatment effectiveness without risking instability or insufficient space. The software can determine the recommended expansion amount using landmarks and reference points to maximize treatment efficiency while keeping the patient comfortable in the transitional dentition.

By determining the recommended expansion amount based on a position of the patient's lower jaw, the software can also provide expansion visualization as a single upper arch treatment or as part of a dual arch treatment. In some embodiments, to determine the recommended expansion amount, the software determines tooth measurements that reflect a distance between tooth cusp midpoints of the first molars. For example, the first tooth cusp midpoint of an upper first molar can be between the lingual distal upper cusp and the lingual mesial upper cusp, and the second tooth cusp midpoint of a lower first molar can be between the buccal distal lower cusp and the buccal mesial lower cusp. In some embodiments, one of the tooth measurements can reflect a distance between the lingual tooth cusp midpoint of the upper right first molar and buccal tooth cusp midpoint of the lower right first molar, and another of the tooth measurements can reflect a distance between the lingual tooth cusp midpoint of the upper left first molar and the buccal tooth cusp midpoint of the lower left first molar. These tooth measurements (and optionally in addition to additional tooth measurements) can be used to determine the recommended expansion amount. The software can determine tooth measurements that reflect the distance between these midpoints for each side of the mouth, and for each treatment stage of a palatal expansion treatment plan. The final treatment stage can correspond to when the tooth measurement (e.g., reflecting either the distance between the lingual tooth cusp midpoint of the upper right first molar and the buccal the buccal tooth cusp midpoint of the lower right first molar, or the distance between the lingual tooth cusp midpoint of the upper left first molar and the buccal tooth cusp midpoint of the lower left first molar) that reflects the smallest distance.

In some embodiments, the software can provide a predicted outcome of the PE treatment. In some embodiments, the software can implement an AI model that provides a predicted outcome of the PE treatment. The AI model can be trained on a set of training data that includes previous PE treatment plans and their outcomes. The current PE treatment, including dental scans of the patient and the optional modifications received from a dental professional, can be provided as input to the trained AI model. The AI model can output a predicted outcome of the palatal expansion treatment, including the predicted actual amount of palatal expansion and the predicted changes to the patient's craniofacial structure. In some embodiments, the software can generate a three-dimensional model of the patient's dentition according the predicted outcome. The software can provide the 3D model of the predicted outcome for display in a user interface (UI) of a user device, e.g., of the dental professional. The 3D model can display the predicted outcome of the palatal expansion of the upper jaw as it relates to the placement of the teeth in the lower jaw. In some embodiments, the predicted outcome can include the predicted palatal shape during and after PE treatment. In some embodiments, the predicted outcome can include the placement of the teeth in the upper jaw during and after the PE treatment. For example, the predicted outcome can include the palatal shape and/or the placement of the teeth at various stages during PE treatment.

In some embodiments, the software can display the PE treatment plan in the UI. In some embodiments, the PE treatment can include multiple treatment stages, and the software can display each of the stages in the UI. Each treatment stage can correspond to a particular dental appliance (e.g., palatal expander or incremental palatal expander in a series of a incremental palatal expanders), and the software can display a visualization of the corresponding dental appliance together with the treatment stage of the PE treatment plan. In some embodiments, the software can display in the UI a visualization of the dentition of the patient and/or a visualization of the dental appliance worn over the dentition of the patient.

In some embodiments, the software can provide planning tools, analytical tools, and/or visualization tools via the UI. In some embodiments, the UI can provide access to various features to enable a dental professional to make modifications to the PE treatment plan, such as modifying the parameters described above. The UI can also include visualization tools, such as measurement and quantitative tools. For example, the UI can enable a dental professional to measure the intercuspal jaw width and the molar buccal overjet. The intercuspal jaw width represents the measurement between the second molars. The molar buccal overjet represents the amount of space that the top molars project over the bottom molars. In some embodiments, the UI can provide access to measurement tool(s) in a grid and/or point-to-point (or ruler) format. In some embodiments, the UI can provide access to analytical tools to enable a dental professional to determine the desired amount of expansion.

In some embodiments, the software can provide a variety of visualization modes in the UI, including, for example, a three-dimensional (3D) view, a two-dimensional (2D) view, and/or a view that is a combination of 3D and 2D. Each viewing mode can accommodate different clinical and/or workflow purposes. In the 3D viewing mode, the software can display 3D model(s) of the patient's craniofacial structure, including, for example, the patient's tooth positioning, the shape of the palate, the arch width, etc. The 3D viewing mode can provide the user (e.g., the dental professional) with the most information on the intended treatment plan. In the 2D viewing mode, the software can display a cross-section of the 3D model. The cross-section can pass through the first molars or other teeth of the patient. The 2D viewing mode can include controls that provide the user with fine control over the position of the cross-section in the mesial-distal direction. The 2D viewing mode can enable a dental professional to identify the desired expansion amount (e.g., optionally using the lower jaw as a reference). In the combined 3D and 2D view, the cross-section view of the 3D model can be displayed next to the full 3D view. In this viewing mode, the 3D view can include a temporary object that illustrates the position of the cross-section surface. This combined 3D and 2D view can provide the user with the most information on the intended treatment plan, while enabling the user to identify the desired expansion using the lower jaw as a reference, for example.

In some embodiments, the software can use 2D cross-sectional planes for PE treatment plan visualization. Such planes may be user selectable and/or adjustable in embodiments. The positioning of the cross-sectional planes with respect to the teeth can provide an informative display of the data and/or of the treatment plan. In some embodiments, the combined UI visualization (e.g., the combined 3D and 2D view) can include a top view (e.g., occlusal view) image of the patient's original scans with lines drawn to represent where the cross-sectional planes intersect the teeth (e.g., the upper and lower first molars), allowing the user (e.g., dental professional) to see all cusps and grooves. In some embodiments, the UI can include visualization of reference points within the mouth, including reference points inside teeth or any other object within the mouth. The reference points can be pre-defined for palatal expansion treatment planning, and/or can be selected by a user (e.g., a dental professional) during treatment planning. In some embodiments, the cross-sectional plane visualization can be visible during the entire expansion treatment planning phase, and can reflect plane position changes during the treatment planning. The cross-sectional plane visualization can include a visualization of both jaws and of both mouth sides simultaneously in some embodiments.

In some embodiments, the cross-sectional plane visualization UI can enable the user to change the position of the cross-sectional plane, e.g., using a sliding bar (also referred to as a slider bar). In some embodiment, the cross-sectional plane visualization UI can include a customizable table that displays different reference point measurements. The reference points (and optionally the reference point measurements) can be visualizable on the 2D cross-sectional image and/or on the occlusal view side teeth images. In some embodiments, two orthogonal projections of the reference points are simultaneously displayed in the UI to make their position in 3D space clear for the user to visualize. In some embodiments, the teeth visualization in the UI can be in color or in grayscale to enable a more contrasting image.

Embodiments described herein provide for an improved method and apparatus for planning and/or visualizing PE treatment that is patient-friendly, time-efficient, and capable of providing consistent and accurate predicted results, thereby enhancing the overall quality of dental care and patient experience. Such improvements in planning and/or visualizing PE treatment are likely to result in increased patient satisfaction as well as improved orthodontic treatment overall. Advantages of the present disclosure and embodiments discussed herein include a more accurate PE prescription, and more accurate orthodontic treatment following PE treatment, thus reducing the time to correctly prescribe and implement palatal expansion and orthodontic remedies. The PE previewer and treatment management tool can use AI to accurately predict the result of PE treatment, avoiding the regular monitoring and prescription mistakes (e.g., the risk of overexpansion) associated with PE treatment. For instance, the digital measurements and quantitative techniques described herein can help a dental practitioner to avoid prescribing over-expansion and/or too frequent monitoring. Additionally, a dental practitioner can use the visualization of upper jaw expansion as a reference for treatment planning of the lower jaw (e.g., in cases of simultaneous lower and upper jaw treatment). In some embodiments, the visualization techniques described herein can help a dental practitioner visualize the vertical clearance of the expander, thus increasing the probability of success for crossbite treatment. In some embodiments, the PE treatment visualization can help a dental practitioner ensure the applicability of the selected device on an early phase of submission, thus avoiding disruptions later in the workflow.

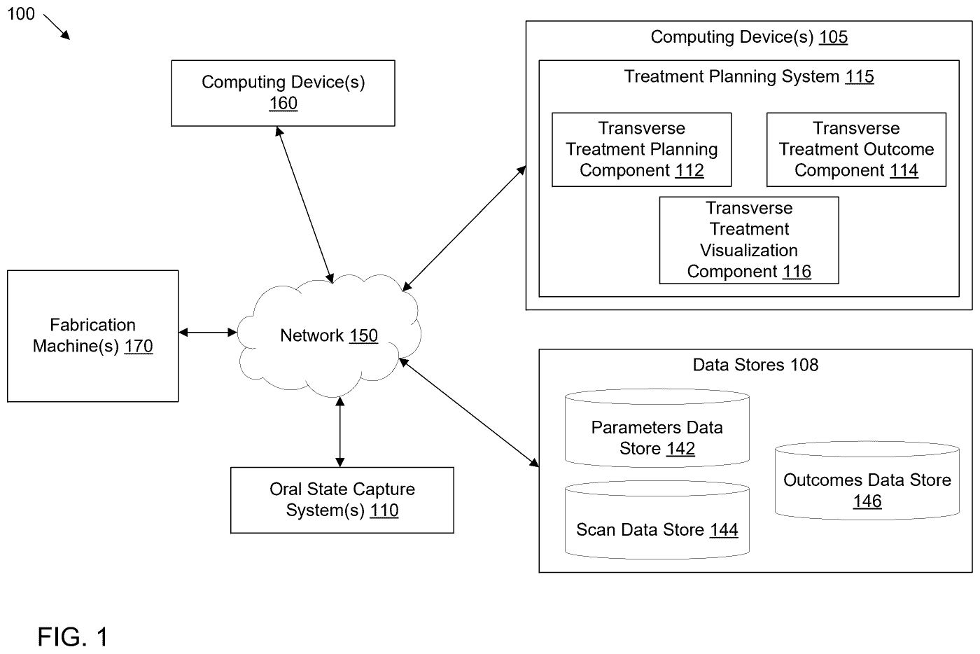

FIG. 1 illustrates a block diagram of an example system 100 for implementing a PE previewer and treatment management tool, in accordance with at least one embodiment of the present disclosure. System 100 includes a computing device 105 that may be coupled to one or more computing devices 160, oral state captures system 110, fabrication machine(s) 170, and/or one or more data stores 108, via a network 150.

Computing devices 105 and/or 160 may each include a processing device, memory, secondary storage, one or more input devices (e.g., such as a keyboard, mouse, tablet, and so on), one or more output devices (e.g., a display, a printer, etc.), and/or other hardware components. Computing device 105 and/or 160 may be connected to one or more data stores 108 either directly or via a network (e.g., network 150). The network 150 may be a local area network (LAN), a public wide area network (WAN) (e.g., the Internet), a private WAN (e.g., an intranet), or a combination thereof. The computing device 105 may additionally or alternatively be connected to computing device(s) 160 and/or oral state capture systems 110 via a network 150, which may be a local area network (LAN), a public wide area network (WAN) (e.g., the Internet), a private WAN (e.g., an intranet), or a combination thereof.

Data store(s) 108 may be or include an internal data store, and/or an external data store that is connected to computing device 105 directly or via a network. Examples of network data stores include a storage area network (SAN), a network attached storage (NAS), and a storage service provided by a cloud computing service provider. Data store(s) 108 may include a file system, a database, or other data storage arrangement.

Fabrication machine(s) 170 may include one or more 3D printers, thermoforming machines, and/or other machines used in the fabrication of palatal expander(s). In some embodiments, fabrication machine(s) 170 can be used to fabricate one or more dental appliances, including, for example, one or more palatal expanders, and/or a series of incremental palatal expanders. In some embodiments, the appliances can be fabricated with one or more materials such as polymer, metal, glass, reinforced fibers, carbon fiber, composites, reinforced composites, aluminum, biological materials, and combinations thereof for example. The appliances can be shaped in many ways, such as with thermoforming or direct fabrication, for example. Alternatively, or in combination, the appliances can be fabricated with machining such as an appliance fabricated from a block of material with computer numeric control machining.

3D printing includes any layer-based additive manufacturing processes. A 3D printer may receive an input of the 3D virtual model of the breakable mold (e.g., as a computer aided drafting (CAD) file or 3D printable file such as a stereolithography file), and may use the 3D virtual model to create the breakable mold. 3D printing may be achieved using an additive process, where successive layers of material are formed in proscribed shapes. 3D printing may be performed using extrusion deposition, granular materials binding, lamination, photopolymerization, or other techniques,

In one embodiment, 3D printing such as stereolithography (SLA), also known as optical fabrication solid imaging, is used to fabricate one or more palatal expanders. In one embodiment, 3D printing such as SLA is used to directly print one or more palatal expanders. In SLA, the palatal expander or mold is fabricated by successively printing thin layers of a photo-curable material (e.g., a polymeric resin) on top of one another. A platform rests in a bath of a liquid photopolymer or resin just below a surface of the bath. A light source (e.g., an ultraviolet laser) traces a pattern over the platform, curing the photopolymer where the light source is directed, to form a first layer of the palatal expander or mold. The platform is lowered incrementally, and the light source traces a new pattern over the platform to form another layer of the palatal expander or mold. This process repeats until the palatal expander or mold is completely fabricated. Each layer may have a thickness of between 25 microns and 200 microns in some embodiments. Once all of the layers of the palatal expander or mold are formed, the palatal expander or mold may be cleaned and cured.

In embodiments where a mold is 3D printed, a palatal expander may be thermoformed over the mold. In one embodiment, a sheet of material is pressure formed or thermoformed over the mold. The sheet may be, for example, a sheet of plastic (e.g., an elastic thermoplastic). To thermoform the palatal expander over the mold, the sheet of material may be heated to a temperature at which the sheet becomes pliable. Pressure may concurrently be applied to the sheet to form the now pliable sheet around the mold. Once the sheet cools, it will have a shape that conforms to the mold. In one embodiment, a release agent (e.g., a non-stick material) is applied to the mold before forming the palatal expander. This may facilitate later removal of the mold from the palatal expander. After thermoforming, the palatal expander may be marked and/or trimmed and removed from the mold. These operations may be performed in different orders, depending on a work flow.

In some embodiments, a palatal expander device can include a shell (e.g., a continuous polymeric shell or a segmented shell) having teeth-receiving cavities that receive and resiliently reposition the teeth. An expander (also referred to as an appliance) or portion(s) thereof may be indirectly fabricated using a physical model of teeth. For example, an appliance (e.g., polymeric appliance) can be formed using a physical model of teeth and a sheet of suitable layers of polymeric material. A “polymeric material,” as used herein, may include any material formed from a polymer. A “polymer,” as used herein, may refer to a molecule composed of repeating structural units connected by covalent chemical bonds often characterized by a substantial number of repeating units (e.g., equal or greater than 3 repeating units, optionally, in some embodiments equal to or greater than 10 repeating units, in some embodiments greater or equal to 30 repeating units) and a high molecular weight (e.g., greater than or equal to 10,000 Da, in some embodiments greater than or equal to 50,000 Da or greater than or equal to 100,000 Da). Polymers are commonly the polymerization product of one or more monomer precursors. The term polymer includes homopolymers, or polymers consisting essentially of a single repeating monomer subunit. The term polymer also includes copolymers which are formed when two or more different types of monomers are linked in the same polymer. Useful polymers include organic polymers or inorganic polymers that may be in amorphous, semi-amorphous, crystalline or semi-crystalline states. Polymers may include polyolefins, polyesters, polyacrylates, polymethacrylates, polystyrenes, polypropylenes, polyethylenes, polyethylene terephthalates, poly lactic acid, polyurethanes, epoxide polymers, polyethers, poly (vinyl chlorides), polysiloxanes, polycarbonates, polyamides, poly acrylonitriles, polybutadienes, poly (cycloolefins), and copolymers. The systems and/or methods provided herein are compatible with a range of plastics and/or polymers. Accordingly, this list is not inclusive, but rather is exemplary. The plastics can be thermosets or thermoplastics. The plastic may be thermoplastic.

The appliance can fit over all teeth present in the upper jaw, or less than all of the teeth. The appliance can be designed specifically to accommodate the teeth of the patient (e.g., the topography of the tooth-receiving cavities matches the topography of the patient's teeth), and may be fabricated based on positive or negative models of the patient's teeth generated by impression, scanning, and the like. Alternatively, the appliance can be a generic appliance configured to receive the teeth, but not necessarily shaped to match the topography of the patient's teeth. Each appliance in a series of appliances in a palatal expansion treatment plan can be configured so a tooth-receiving cavity has a geometry corresponding to an intermediate or final palatal expansion amount intended for the appliance. The patient's palatal suture can be progressively expanded from an initial position to a target position by placing a series of incremental palatal expansion position appliances over the patient's teeth.

In some embodiments, an appliance can be produced using indirect fabrication techniques, such as thermoforming over a positive or negative mold, which may be inspected using the methods and systems described herein above. Indirect fabrication of an orthodontic appliance can involve producing a positive or negative mold of the patient's dentition in a target arrangement (e.g., by rapid prototyping, milling, etc.) and thermoforming one or more sheets of material over the mold in order to generate an appliance shell. In an example of indirect fabrication, a mold of a patient's dental arch may be fabricated from a digital model of the dental arch, and a shell may be formed over the mold (e.g., by thermoforming a polymeric sheet over the mold of the dental arch and then trimming the thermoformed polymeric sheet). The fabrication of the mold may be formed by a rapid prototyping machine (e.g., a SLA 3D printer). The rapid prototyping machine may receive digital models of molds of dental arches and/or digital models of the appliances after the digital models of the appliances have been processed by processing logic of a computing device. The processing logic may include hardware (e.g., circuitry, dedicated logic, programming logic, microcode, etc.), software (e.g., instructions executed by a processing device), firmware, or a combination thereof.

To manufacture the molds, a shape of a dental arch for a patient at a treatment stage is determined based on a expansion treatment plan, e.g., as determined by transverse treatment planning component 112. For example, as described throughout, the treatment plan may be generated based on an intraoral scan of a dental arch to be molded. The intraoral scan of the patient's dental arch may be performed to generate a three dimensional (3D) virtual model of the patient's dental arch (mold). For example, a full scan of the mandibular and/or maxillary arches of a patient may be performed to generate 3D virtual models thereof. The intraoral scan may be performed by creating multiple overlapping intraoral images from different scanning stations and then stitching together the intraoral images to provide a composite 3D virtual model. In other applications, virtual 3D models may also be generated based on scans of an object to be modeled or based on use of computer aided drafting technologies (e.g., to design the virtual 3D mold). Alternatively, an initial negative mold may be generated from an actual to be modeled (e.g., a dental impression or the like). The negative mold may then be scanned to determine a shape of a positive mold that will be produced.

Once the virtual 3D model of the patient's dental arch is generated, a dental practitioner and/or transverse treatment planning component 112 may determine a desired treatment outcome, which includes final palatal expansion of the patient's upper arch. Processing logic may then determine a number of treatment stages to cause the expansion starting position to the target final position. The shape of the final virtual 3D model and each intermediate virtual 3D model may be determined by computing the progression of palatal expansion throughout palatal expansion treatment from initial position to final position. For each treatment stage, a separate virtual 3D model will be different. The original virtual 3D model, the final virtual model 3D model, and each intermediate virtual 3D model is unique and customized to the patient.

Accordingly, multiple different virtual 3D models (digital designs) of a dental arch may be generated for a single patient. A first virtual 3D model may be a unique model of a patient's dental arch and/or teeth as they presently exist, and a final virtual 3D may be a model of the patient's dental arch and/or teeth after the palatal expansion treatment plan. Multiple intermediate virtual 3D models may be modeled, each of which may be incrementally different from previous virtual 3D models.

Each virtual 3D model of a patient's dental arch may be used to fabricate customized physical mold of the dental arch at a particular stage of treatment and/or to fabricate a dental appliance (e.g., a palatal expander, an orthodontic aligner, etc.). In some embodiments, a mold is fabricated (e.g., 3D printed), and a dental appliance is thermoformed over the mold from a sheet of material e.g., plastic). In some embodiments, the dental appliance is directly fabricated (e.g., directly 3D printed). The shape of the mold and/or dental appliance may be at least in part based on the shape of the virtual 3D model for that treatment stage. The virtual 3D model may be represented in a file such as a computer aided drafting (CAD) file or a 3D printable file such as a stereolithography (STL) file. The virtual 3D model for the mold or dental appliance may be sent to a third party (e.g., clinician office, laboratory, manufacturing facility, or other entity). The virtual 3D model may include instructions that will control a fabrication system or device in order to produce the mold or dental appliance with specific geometries.

A clinician office, laboratory, manufacturing facility or other entity may receive the virtual 3D model of the mold or dental appliance, the digital model having been created as set forth above. The entity may input the digital model into a 3D printer (e.g., a rapid prototyping machine). The rapid prototyping machine then manufactures the mold using the digital model. 3D printing includes any layer-based additive manufacturing processes. 3D printing may be achieved using an additive process, where successive layers of material are formed in proscribed shapes. 3D printing may be performed using extrusion deposition, granular materials binding, lamination, photopolymerization, continuous liquid interface production (CLIP), or other techniques. 3D printing may also be achieved using a subtractive process, such as milling.

In some instances SLA is used to fabricate an SLA mold or dental appliance. In SLA, the mold or dental appliance is fabricated by successively printing thin layers of a photo-curable material (e.g., a polymeric resin) on top of one another. A platform rests in a bath of liquid photopolymer or resin just below a surface of the bath. A light source (e.g., an ultraviolet laser) traces a pattern over the platform, curing the photopolymer where the light source is directed, to form a first layer of the mold or dental appliance. The platform is lowered incrementally, and the light source traces a new pattern over the platform to form another layer of the mold or dental appliance at each increment. This process repeats until the mold or dental appliance is completely fabricated. Once all of the layers of the mold or dental appliance are formed, the mold or dental appliance may be cleaned and cured.

Materials such as polyester, a co-polyester, a polycarbonate, a thermopolymeric polyurethane, a polypropylene, a polyethylene, a polypropylene and polyethylene copolymer, an acrylic, a cyclic block copolymer, a polyetheretherketone, a polyamide, a polyethylene terephthalate, a polybutylene terephthalate, a polyetherimide, a polyethersulfone, a polytrimethylene terephthalate, a styrenic block copolymer (SBC), a silicone rubber, an elastomeric alloy, a thermopolymeric elastomer (TPE), a thermopolymeric vulcanizate (TPV) elastomer, a polyurethane elastomer, a block copolymer elastomer, a polyolefin blend elastomer, a thermopolymeric co-polyester elastomer, a thermopolymeric polyamide elastomer, or combinations thereof, may be used to directly form the mold or dental appliance. The materials used for fabrication of the mold or dental appliance can be provided in an uncured form (e.g., as a liquid, resin, powder, etc.) and can be cured (e.g., by photopolymerization, light curing, gas curing, laser curing, crosslinking, etc.). The properties of the material before curing may differ from the properties of the material after curing.

After the mold or dental appliance is generated, it may be inspected using the systems and/or methods described herein above. If the mold passes the inspection, then it may be used to form an appliance (e.g., an expander).

Appliances may be formed from each mold or may be directly printed. When applied to the teeth of the patient, the dental appliances may provide forces to expand the patient's palate as dictated by the treatment plan. The shape of each appliance is unique and customized for a particular patient and a particular treatment stage. In an example, the appliances in the treatment plan can be pressure formed or thermoformed over the molds. Each mold may be used to fabricate an appliance that will apply forces to the patient's teeth at a particular stage of the orthodontic treatment. The appliances in the treatment plan can each have teeth-receiving cavities that receive and resiliently reposition the teeth in accordance with a particular treatment stage.

In one embodiment, a sheet of material is pressure formed or thermoformed over the mold. The sheet may be, for example, a sheet of polymeric (e.g., an elastic thermopolymeric, a sheet of polymeric material, etc.). To thermoform the shell over the mold, the sheet of material may be heated to a temperature at which the sheet becomes pliable. Pressure may concurrently be applied to the sheet to form the now pliable sheet around the mold. Once the sheet cools, it will have a shape that conforms to the mold. In one embodiment, a release agent (e.g., a non-stick material) is applied to the mold before forming the shell. This may facilitate later removal of the mold from the shell.

Additional information may be added to the appliance. The additional information may be any information that pertains to the expander. Examples of such additional information includes a part number identifier, patient name, a patient identifier, a case number, a sequence identifier (e.g., indicating which stage a particular expander is in the treatment sequence), a date of manufacture, a clinician name, a logo, and so forth. For example, after an appliance is thermoformed, the expander may be laser marked with a part number identifier (e.g., serial number, barcode, or the like).

In some embodiments, the system may be configured to read (e.g., optically, magnetically, or the like) an identifier (barcode, serial number, electronic tag or the like) of the mold to determine the part number associated with the expander formed thereon. After determining the part number identifier, the system may then tag the expander with the unique part number identifier. The part number identifier may be computer readable and may associate that expander to a specific patient, to a specific stage in the treatment sequence, a digital model representing the mold the expander was manufactured from, and/or a digital file including a virtually generated digital model or approximated properties thereof of that expander (e.g., produced by approximating the outer surface of the expander based on manipulating the digital model of the mold, inflating or scaling projections of the mold in different planes, etc.).

After an appliance is formed over a mold for a treatment stage, that appliance is subsequently trimmed along a cutline (also referred to as a trim line) and the appliance may be removed from the mold. The processing logic may determine a cutline for the appliance. The determination of the cutline(s) may be made based on the virtual 3D model of the dental arch at a particular treatment stage, based on a virtual 3D model of the appliance to be formed over the dental arch, or a combination of a virtual 3D model of the dental arch and a virtual 3D model of the appliance. The location and shape of the cutline can be important to the functionality of the appliance (e.g., an ability of the appliance to apply desired forces to a patient's teeth) as well as the fit and comfort of the appliance. In some embodiments, the cut line may be modified in the digital design of the appliance as one of the corrective actions taken when a probable point of damage is determined to exist in the digital design of the appliance. In some embodiments, the cutline may be a straight line across the appliance at the gingival line, below the gingival line, or above the gingival line. In some embodiments, the cutline may be a gingival cutline that represents an interface between an appliance and a patient's gingiva. In such embodiments, the cutline controls a distance between an edge of the appliance and a gum line or gingival surface of a patient.

Each patient has a unique dental arch with unique gingiva. Accordingly, the shape and position of the cutline may be unique and customized for each patient and for each stage of treatment. For instance, the cutline is customized to follow along the gum line (also referred to as the gingival line). In some embodiments, the cutline may be away from the gum line in some regions and on the gum line in other regions. For example, it may be desirable in some instances for the cutline to be away from the gum line (e.g., not touching the gum) where the shell will touch a tooth and on the gum line (e.g., touching the gum) in the interproximal regions between teeth. Accordingly, it is important that the shell be trimmed along a predetermined cutline.

In some embodiments, the expander appliances herein (or portions thereof) can be produced using direct fabrication, such as additive manufacturing techniques (also referred to herein as “3D printing) or subtractive manufacturing techniques (e.g., milling). In some embodiments, direct fabrication involves forming an object (e.g., an orthodontic appliance or a portion thereof) without using a physical template (e.g., mold, mask etc.) to define the object geometry. Additive manufacturing techniques can be categorized as follows: (1) vat photopolymerization (e.g., stereolithography), in which an object is constructed layer by layer from a vat of liquid photopolymer resin; (2) material jetting, in which material is jetted onto a build platform using either a continuous or drop on demand (DOD) approach; (3) binder jetting, in which alternating layers of a build material (e.g., a powder-based material) and a binding material (e.g., a liquid binder) are deposited by a print head; (4) fused deposition modeling (FDM), in which material is drawn though a nozzle, heated, and deposited layer by layer; (5) powder bed fusion, including but not limited to direct metal laser sintering (DMLS), electron beam melting (EBM), selective heat sintering (SHS), selective laser melting (SLM), and selective laser sintering (SLS); (6) sheet lamination, including but not limited to laminated object manufacturing (LOM) and ultrasonic additive manufacturing (UAM); and (7) directed energy deposition, including but not limited to laser engineering net shaping, directed light fabrication, direct metal deposition, and 3D laser cladding. For example, stereolithography can be used to directly fabricate one or more of the appliances. In some embodiments, stereolithography involves selective polymerization of a photosensitive resin (e.g., a photopolymer) according to a desired cross-sectional shape using light (e.g., ultraviolet light). The object geometry can be built up in a layer-by-layer fashion by sequentially polymerizing a plurality of object cross-sections. As another example, the appliances can be directly fabricated using selective laser sintering. In some embodiments, selective laser sintering involves using a laser beam to selectively melt and fuse a layer of powdered material according to a desired cross-sectional shape in order to build up the object geometry. As yet another example, the appliances can be directly fabricated by fused deposition modeling. In some embodiments, fused deposition modeling involves melting and selectively depositing a thin filament of thermoplastic polymer in a layer-by-layer manner in order to form an object. In yet another example, material jetting can be used to directly fabricate the appliances. In some embodiments, material jetting involves jetting or extruding one or more materials onto a build surface in order to form successive layers of the object geometry.

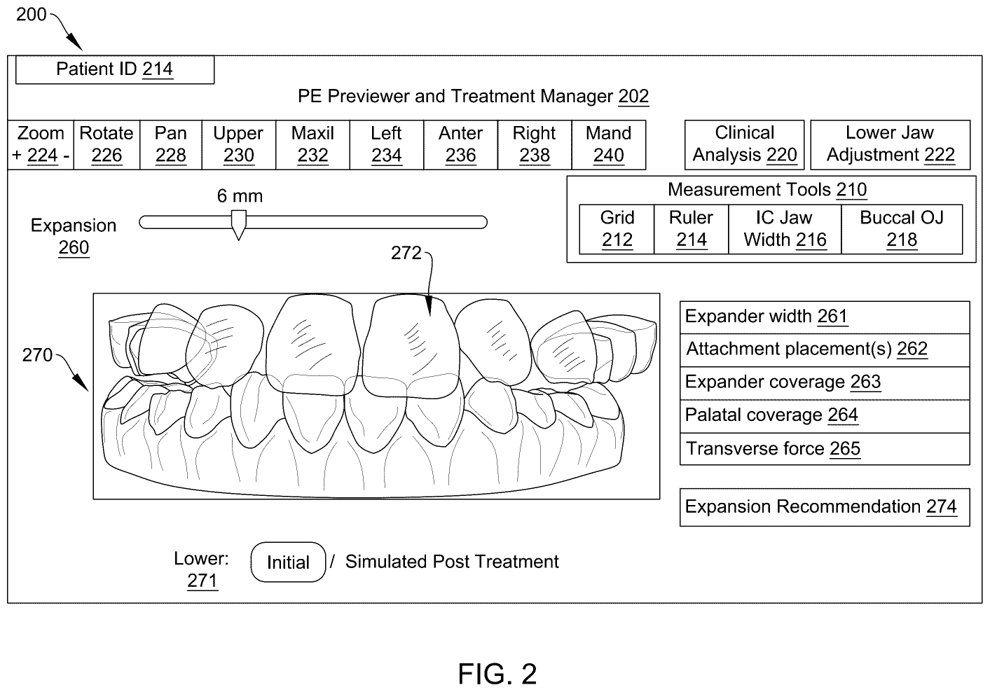

In some embodiments, data stores 108 can include a parameters data store 142, a scan data store 144, and/or an outcomes data store 146. In some embodiments, data stored in parameters data store 142, scan data store 144, and/or outcomes data store 146 can be separated into additional data stores, and/or combined into a single data store. Parameters data store 142 can include parameters for PE treatment plans, such as expansion amount, number of expanders, expander wear-time, and/or other variables for a PE treatment plan. In some embodiments, the parameters data store 142 can store parameters provided by an AI model, e.g., as a recommended PE treatment plan. In some embodiments, the parameters data store 142 can store parameters received from a user device (e.g., computing device 160). For example, a dental professional can provide PE treatment plan parameters for a particular patient (e.g., via UI 200 of FIG. 2). Outcomes data store 146 can include data corresponding to predicted outcome of a PE treatment plan, e.g., as provided by an AI model. Scan data store 144 can include scan data of a patient, including, for example, image data corresponding to one or more scans (e.g., intraoral scan(s), CBCT scan(s), etc.), corresponding to one or more x-rays (e.g., panoramic x-rays), corresponding to one or more photographs (e.g., generated by a camera and/or a smart phone), corresponding to one or more sensors (e.g., generated by a compliance indicator, and/or by the a sensor embedded on or with a palatal expander), and/or other types of image data of a patient's dental arch(es), mouth, face, and/or craniofacial structure. In some embodiments, scan data can also include segmentation data, registration data, and/or other data corresponding to scan data of the patient. In some embodiments, scan data can also include patient data, such as patient's demographics, such as biological age, gender, dental age, ethnicity, genetic profiles, etc. In some embodiments, scan data can include data generated by the oral state capture system 110. In some embodiments, scan data can be used to generate a virtual model (e.g., a virtual 2D model or a virtual 3D model) of a patient's craniofacial structure.

In some embodiments, oral state capture system(s) 110 can include an intraoral scanner, a CBCT scanner (and/or another imaging device, such as a CT scanner), an electronic compliance indicator (ECI) device, sensors on a palatal expander, a camera, a video camera, and/or optionally a computing device. In some embodiments, the computing device can be part of a scanner in the oral state capture system 110. In some embodiments, the computing device can be part of computing device 160, 105, and/or a separate device (not shown), and the oral state capture system 110 can send captured data (e.g., scan data, image data, video data) for processing on a separate device. in some embodiments, the oral state capture system 110 can include a patient or client device that can take 2D or 3D images and/or videos of the patient's anatomy in a non-clinical setting (e.g., at a patient's home). For example, the oral state capture system 110 may include a mobile computing device such as a mobile phone or table computer of the user. The oral state capture system 110 can obtain scan data, which may be stored in scan data store 144. In some embodiments, the data captured can be used to reconstruct a 3D image of the patient's teeth, mouth, jaw, neck, ear, nose and/or throat.

In some embodiments, oral state capture system 110 includes a CBCT machine. A CBCT machine is a type of x-ray machine that uses a cone-shaped x-ray beam to capture data about the patient's anatomy. The CBCT scan can generate multiple (e.g., 150-200) images from a variety of angles.

In some embodiments, oral state capture system 110 includes an intraoral scanning system comprising a scanner for obtaining intraoral scans (e.g., 3D data) of a patient's dentition and optionally a computing device. Alternatively, oral state capture system 110 may include an intraoral scanner, and the computing device may connect to the intraoral scanner to effectuate intraoral scanning. In embodiments, the computing device or another computing device of oral state capture system 110 includes an intraoral scan application that processes intraoral scans generated by the intraoral scanner to generate 3D models of the patient's upper and/or lower dental arches.

In some embodiments, the intraoral scanner may include a probe (e.g., a hand held probe) for optically capturing three-dimensional structures. The intraoral scanner may be used to perform an intraoral scan of a patient's oral cavity. An intraoral scan application running on a computing device may communicate with the scanner to effectuate the intraoral scan. A result of the intraoral scan may be scan data (e.g., of scan data store 144) that may include one or more sets of intraoral scans, which may include intraoral images. Each intraoral scan may include a two-dimensional (2D) or 3D image that may include depth information (e.g., a height map) of a portion of a dental site. In embodiments, intraoral scans include x, y and z information. In one embodiment, the intraoral scanner generates numerous discrete (i.e., individual) intraoral scans.

In some embodiments, the oral state capture system 110 can include an electronic compliance indicator (ECI) device. In some embodiments, the ECI device can be used to accurately monitor a patient's compliance to a prescribed oral appliance (e.g., palatal expander) schedule. For instance, a palatal expander that is ECI-capable can have one or more sensors designed to detect temperature and/or proximity to a patient's tooth. The sensors can pair to a mobile phone, e.g., via a Bluetooth®-enabled “smart” aligner or expander case, and can receive and/or transmit data between the mobile phone and the ECI. In some embodiments, the ECI device can include a pressure sensor that can measure pressure and can convert the measured physical pressure exerted on it into an electrical signal. The pressure sensor on the occlusal surface of the teeth can detect the occlusal force or biting pressure, which can be used to detect bruxism (grinding and/or clenching of the teeth). The pressure sensor can include a sensing element that directly responds to pressure, a transducer that converts the physical change in the sensing element into an electrical signal, a signal conditioning component that can amplify, filter, and/or convert the signal into a digital signal, and/or an output component that can transmit the conditioned signal to a processing device. For example, the pressure sensor can be used to measure and analyze the forces exerted during various dental procedures and treatments, such as occlusal analysis, implantology, orthodontics, prosthodontics, and/or periodontology. In some embodiments, the pressure sensor can measure electrical activity recorded during execution of a sequence of actions (e.g., bruxism-related events such as teeth clenching and teeth grinding, etc., and/or bruxism-unrelated events such as swallowing, lightly nodding the head, lightly shaking the head, speaking, etc.). In some embodiments, the pressure sensor can record a time-averaged value during execution of a particular sequence of actions. The pressure sensor can detect, record, and/or transmit signals to the computing device. The pressure data (e.g., the detected signals) can indicate clenching or grinding of a patient. In some embodiments, the pressure sensor can be attached to a processing device in oral state capture system 110, or can be otherwise connected to a processing device in oral state capture system 110.

In some embodiments, a palatal expander can include force sensor, a pressure sensor, a displacement sensor, and/or other electronic sensor. A force sensor or pressure sensor within the palatal region of the palatal expander can be used to measure force reduction during the holding period. A force sensor or pressure sensor within the buccal region of the palatal expander can be used to measure the force to insert or remove the palatal expander from an upper dental arch of a patient. A displacement sensor on the occlusal region of the palatal expander can be used to assess fit of the palatal expander. A rotational sensor in a palatal region of the palatal expander can be used to detect rotation of one region of the palate relative to another region of the palate (e.g., of a left side of the palate relative to a right side of the palate). One or more rotational sensors in tooth regions of the palatal expander can be used to detect rotation of teeth (e.g., of molars). One or more force (or pressure) sensors may be disposed in one or more regions (e.g., tooth regions) of the palatal expander, and may be used to detect bite force on the palatal expander.

In some embodiments, oral state capture system 110 is connected to data store(s) 108 either directly or via network 150. In some embodiments, oral state capture system 110 transmits image data (e.g., CBCT scan data, intraoral scan data, images) and/or video recording data to data store 108 for storage therein.

According to an example, a user (e.g., a practitioner) may subject a patient to intraoral scanning. In doing so, the user may apply an intraoral scanner to one or more patient intraoral locations. The scanning may be divided into one or more segments (also referred to as roles). As an example, the segments may include a lower dental arch of the patient, an upper dental arch of the patient, one or more preparation teeth of the patient (e.g., teeth of the patient to which a dental device such as a crown or other dental prosthetic will be applied), one or more teeth which are contacts of preparation teeth (e.g., teeth not themselves subject to a dental device but which are located next to one or more such teeth or which interface with one or more such teeth upon mouth closure), and/or patient bite (e.g., scanning performed with closure of the patient's mouth with the scan being directed towards an interface area of the patient's upper and lower teeth). Via such scanner application, the intraoral scanner may provide scan data to computing device 105 (or to another computing device of oral state capture system 110). The scan data may be provided in the form of intraoral scan data sets, each of which may include 2D intraoral images (e.g., color 2D images) and/or 3D intraoral scans of particular teeth and/or regions of an intraoral site. In one embodiment, separate intraoral scan data sets are created for the maxillary arch, for the mandibular arch, for a patient bite, and/or for each preparation tooth. Alternatively, a single large intraoral scan data set is generated (e.g., for a mandibular and/or maxillary arch). Intraoral scans may be provided from the intraoral scanner to the computing device 105 (or other computing device) in the form of one or more points (e.g., one or more pixels and/or groups of pixels). For instance, the intraoral scanner may provide an intraoral scan as one or more 3D point clouds. The intraoral scans may each comprise height information.

The manner in which the oral cavity of a patient is to be scanned may depend on the procedure to be applied thereto. For example, if an upper or lower denture is to be created, then a full scan of the mandibular or maxillary edentulous arches may be performed. In contrast, if a bridge is to be created, then just a portion of a total arch may be scanned which includes an edentulous region, the neighboring preparation teeth (e.g., abutment teeth) and the opposing arch and dentition. Alternatively, full scans of upper and/or lower dental arches may be performed if a bridge is to be created.

By way of non-limiting example, dental procedures may be broadly divided into prosthodontic (restorative) and orthodontic procedures, and then further subdivided into specific forms of these procedures. Additionally, dental procedures may include identification and treatment of gum disease, sleep apnea, and intraoral conditions such as malocclusions, temporomandibular joint disorder (TMD), gingival recession, tooth grinding, and so on. The term prosthodontic procedure refers, inter alia, to any procedure involving the oral cavity and directed to the design, manufacture or installation of a dental prosthesis at a dental site within the oral cavity (intraoral site), or a real or virtual model thereof, or directed to the design and preparation of the intraoral site to receive such a prosthesis. A prosthesis may include any restoration such as crowns, veneers, inlays, onlays, implants and bridges, for example, and any other artificial partial or complete denture. The term orthodontic procedure refers, inter alia, to any procedure involving the oral cavity and directed to the design, manufacture or installation of orthodontic elements at an intraoral site within the oral cavity, or a real or virtual model thereof, or directed to the design and preparation of the intraoral site to receive such orthodontic elements. These elements may be appliances including but not limited to brackets and wires, retainers, clear aligners, expanders, or functional appliances.

In embodiments, intraoral scanning may be performed on a patient's oral cavity during a visitation of a dental office. The intraoral scanning may be performed, for example, as part of a semi-annual or annual dental health checkup. The intraoral scanning may also be performed before, during and/or after one or more dental treatments, such as orthodontic treatment and/or prosthodontic treatment. The intraoral scanning may be a full or partial scan of the upper and/or lower dental arches, and may be performed in order to gather information for performing dental diagnostics, to generate a treatment plan, to determine progress of a treatment plan, and/or for other purposes. The scan data generated from the intraoral scanning may include 3D scan data, 2D color images, NIR (near infrared) and/or infrared images, and/or ultraviolet images, of all or a portion of the upper jaw and/or lower jaw. The scan data may further include one or more intraoral scans showing a relationship of the upper dental arch to the lower dental arch. These intraoral scans may be usable to determine a patient bite and/or to determine occlusal contact information for the patient. The patient bite may include determined relationships between teeth in the upper dental arch and teeth in the lower dental arch.

Intraoral scanners may work by moving the intraoral scanner inside a patient's mouth to capture all viewpoints of one or more tooth. During scanning, the intraoral scanner is calculating distances to solid surfaces in some embodiments. Each intraoral scan is overlapped algorithmically, or ‘stitched’, with the previous set of scans to generate a growing 3D surface. As such, each scan is associated with a rotation in space, or a projection, to how it fits into the 3D surface.

During intraoral scanning, an intraoral scan application (e.g., executing on computing device 105 or a computing device of oral state capture system 110) may register and stitch together two or more intraoral scans generated thus far from the intraoral scan sessions. In one embodiment, performing registration includes capturing 3D data of various points of a surface in multiple scans, and registering the scans by computing transformations between the scans. One or more 3D surfaces may be generated based on the registered and stitched together intraoral scans during the intraoral scanning. The one or more 3D surfaces may be output to a display so that a doctor or technician can view their scan progress thus far. As each new intraoral scan is captured and registered to previous intraoral scans and/or a 3D surface, the one or more 3D surfaces may be updated, and the updated 3D surface(s) may be output to the display. In embodiments, separate 3D surfaces are generated for the upper jaw and the lower jaw. This process may be performed in real time or near-real time to provide an updated view of the captured 3D surfaces during the intraoral scanning process.

When a scan session or a portion of a scan session associated with a particular scanning role (e.g., upper jaw role, lower jaw role, bite role, etc.) is complete (e.g., all scans for an intraoral site or dental site have been captured), the intraoral scan application may automatically generate a virtual 3D model of one or more scanned dental sites (e.g., of an upper jaw and a lower jaw). The final 3D model(s) may each be a set of 3D points and their connections with each other (i.e., a mesh). To generate a virtual 3D model, the intraoral scan application may register and stitch together the intraoral scans generated from the intraoral scan session that are associated with a particular scanning role. The registration performed at this stage may be more accurate than the registration performed during the capturing of the intraoral scans, and may take more time to complete than the registration performed during the capturing of the intraoral scans. In one embodiment, performing scan registration includes capturing 3D data of various points of a surface in multiple scans, and registering the scans by computing transformations between the scans. The 3D data may be projected into a 3D space of a 3D model to form a portion of the 3D model. The intraoral scans may be integrated into a common reference frame by applying appropriate transformations to points of each registered scan and projecting each scan into the 3D space.

In one embodiment, registration is performed for adjacent or overlapping intraoral scans (e.g., each successive frame of an intraoral video). Registration algorithms are carried out to register two adjacent or overlapping intraoral scans (e.g., two adjacent blended intraoral scans) and/or to register an intraoral scan with a 3D model, which essentially involves determination of the transformations which align one scan with the other scan and/or with the 3D model. Registration may involve identifying multiple points in each scan (e.g., point clouds) of a scan pair (or of a scan and the 3D model), surface fitting to the points, and using local searches around points to match points of the two scans (or of the scan and the 3D model). For example, the intraoral scan application may match points of one scan with the closest points interpolated on the surface of another scan, and iteratively minimize the distance between matched points. Other registration techniques may also be used. Registration data can be stored in data store 108 as a portion of scan data store 144, in embodiments.

The intraoral scan application may repeat registration for all intraoral scans of a sequence of intraoral scans to obtain transformations for each intraoral scan, to register each intraoral scan with previous intraoral scan(s) and/or with a common reference frame (e.g., with the 3D model). The intraoral scan application may integrate intraoral scans into a single virtual 3D model (or two virtual 3D models, one for each dental arch) by applying the appropriate determined transformations to each of the intraoral scans. Each transformation may include rotations about one to three axes and translations within one to three planes.

The generated virtual 3D model can include color information. In some embodiments, the scan data can include color information, e.g., from 2D color images captured during the scanning process. The oral state capture system 110 can use the color information to add color texture to the 3D model(s). Once virtual 3D model(s) of the patient's dental arches are generated, they may be stored in data store 108 as a portion of scan data store 144 in embodiments.

In some embodiments, computing device 105 and/or computing device 160 is a desktop computer, a laptop computer, a server computer, etc., located at a doctor office. In some embodiments, computing device 105 is a server computing device (e.g., of a data center) that may be accessed from client devices (e.g., client devices of doctors, patients, etc.). In one embodiment, computing device 160 is a client device. In some embodiments, computing device 105 is a virtual machine. For example, computing device 105 may be a virtual machine that runs in a cloud computing environment.

In some embodiments, computing device 105 can include a treatment planning system 115, which can include a transverse treatment planning component 112, a transverse treatment outcome component 114, and/or a transverse treatment visualization component 116. Treatment planning system 115 can include software, hardware, and/or firmware configured to perform one or more operations with respect to generating and/or displaying a PE previewer and treatment planning tool.

In some embodiments, the transverse treatment planning component 112 can receive or identify scan data for a particular patient. In some embodiments, the scan data can be stored in scan data store 144. The scan data can include intraoral scan data, e.g., captured from an intraoral scanning device of oral state capture system 110. An intraoral scanner can include a probe (e.g., a hand held probe) for optically capturing three-dimensional structures. The intraoral scanner can be used to perform an intraoral scan of a patient's oral cavity. An intraoral scan application (e.g., running on computing device 105) can communicate with the scanner to effectuate the intraoral scan. A result of the intraoral scan may be scan data 144 that may include one or more sets of intraoral scans, which may include intraoral images. Each intraoral scan may include a two-dimensional (2D) or 3D image. In embodiments, intraoral scans include x, y and z information. In one embodiment, the intraoral scanner generates numerous discrete (i.e., individual) intraoral scans. In some embodiments, the intraoral scan data can include information indicating the shape of the palate, including the width and depth of the palate.

In some embodiments, the transverse treatment planning component 112 can process scan data, image data, etc. of a patient's dentition to provide a recommended PE treatment plan for the patient. In some embodiments, the transverse treatment planning component 112 can include and/or implement an AI model that is trained to output a recommended PE treatment plan for a particular patient. In some embodiments, the AI model can be trained using a training dataset that includes historical palatal expander-treated cases. The training dataset can include upper arch and/or palatal 3D data before the palatal expander treatment, and upper arch and/or palatal 3D data after the palatal expander treatment. The training data set can include, for example, scan data of a patient before palatal expander treatment (including, for example, the arch width, the palatal shape, etc.), the planned expansion amount of the palatal expander treatment, scan data following the palatal expander treatment (including, e.g., the arch width, the palatal shape, etc.), the number of expanders worn during the palatal expander treatment, the prescribed amount of expansion for the palatal expander treatment, the actual expansion amount following the palatal expander treatment, and/or any other relevant information of the palatal expander treatment. Using the training dataset, the AI model can be trained to provide an expansion amount for a particular palatal shape and width, as indicated in the patient's scan data.

In some embodiments, the transverse treatment planning component 112 can provide scan data of a patient as input to the AI model, and the AI model can output a recommended PE treatment plan. The PE treatment plan can include, for example, a recommended expansion amount and/or a number of palatal expanders to prescribe (e.g., to achieve the recommended expansion amount). In some embodiments, the transverse treatment planning component 112 can identify PE treatment plan parameter values. In some embodiments, the treatment plan parameter values can be specific to the dental professional. That is, a particular dental professional may prefer to prescribe more aggressive PE treatments, as compared to other dental professionals. The PE treatment plan parameter values can be provided as additional input to the AI model, to receive a recommended PE treatment plan that is tailored to the particular dental professional. In some embodiments, the PE treatment plan parameters can be specific to the region in which the patient lives and/or dental professional practices (e.g., one region may prefer less palatal expansion when compared to other regions). In some embodiments, the transverse treatment planning component 112 can analyze the dental professional's prior PE treatment plans (including the outcome of the treatment plans) to identify PE treatment plan parameter values for that dental professional. The PE treatment plan parameter values can be stored in parameter data store 142.

In some embodiments, the transverse treatment planning component 112 can generate a 3D dental model based on the scan data of the patient, e.g., using techniques described herein. The 3D dental model can include an initial breadth of the palate of the patient. The breadth of the palate can represent the width dimension of the palate, e.g., as measured across the mouth from one molar to the opposite molar. The transverse treatment planning component 112 can generate a palatal expansion treatment plan that includes a series of breadths of the palate corresponding to a progressive expansion of the palate from the initial breadth toward a target breadth. The target breadth can correspond to the target expansion amount following the palatal expansion treatment plan. In some embodiments, the target breadth can correspond to an intermediate breadth during the palatal expansion treatment plan. In some embodiments, to generate the palatal expansion treatment plan, the transverse treatment planning component 112 can process the 3D dental model to obtain the target breadth of the palate, and can identify one or more intermediate breadths in the series of breadths corresponding to the progressive expansion of the palate. For example, the transverse treatment planning component 112 can provide the 3D dental model (and/or scan data of the patient) to an AI model that is trained to provide a target breadth, and optionally to provide a series of intermediate breadths. In some embodiments, the AI model can be trained to provide the target breadth, and the transverse treatment planning component 112 can determine the series of intermediate breadths, e.g., using the transverse force of the palatal expanders and the amount of expansion between the initial breadth and the target breadth.

In some embodiments, the transverse treatment planning component 112 can receive treatment plan parameters from a user (e.g., a dental professional using computing device 160), e.g., via the UI. In some embodiments, the transverse treatment planning component 112 can store data of historical PE treatment cases of the dental professional (e.g., in data stores 108), and can determine a trend for treatment plan parameters of the dental professional. In some embodiments, the transverse treatment planning component 112 can analyze the historical PE treatment cases of the dental professional to determine a pattern of treatment. In some embodiments, the transverse treatment planning component 112 can provide the historical PE treatment cases of the dental professional as additional input to the trained AI model, and the AI model can tailor its output based on the historical PE treatment cases of the dental professional. For example, the transverse treatment planning component 112 can provide the dental professional's historical PE treatment case data as context, and/or as part of the prompt provided to the AI model. In some embodiments, the dental professional's historical PE treatment case data can be used to fine-tune the AI model to the dental professional. Thus, the PE treatment plan provided by the AI model can be tailored to the dental professional. In some embodiments, a subset of the historical PE treatment cases can be used to tailor the PE treatment plan. For example, the dental professional can have ten years worth of historical PE treatment cases, and the dental professional can limit the historical PE treatment cases from the past two years. As another example, the subset of historical PE treatment cases can include cases that are similar to the current case (e.g., similar age group of the patient). In some embodiments, the treatment planning system 115 can provide filtering tools via the UI for the dental professional to select which cases of the dental professional's historical PE treatment cases to use to tailor the PE treatment plan provided by the AI model. In some embodiments, the transverse treatment planning component 112 can automatically determine which cases of the dental professional's historical PE treatment cases to use to tailor the PE treatment plan provided by the AI model, e.g., based on one or more criteria.

In some embodiments, the transverse treatment planning component 112 can identify values of one or more additional parameters that are associated with a particular patient and/or associated with a particular dental practitioner, e.g., as stored in parameters data store 142. Optionally, the transverse treatment planning component 112 can receive a modification of one or more of the parameter values. For example, a dental professional can modify one or more of the parameter values for the particular patient. The parameters can include, for example, the amount of expansion, the vertical clearance of the expander (e.g., the “width” of the expander), the attachment placement on teeth, which teeth are covered by the expander, which part of the palate is covered by the expander, the amount of transverse force applied by the expander, etc. The parameter values can be used to create the PE treatment plan for the particular patient.

In some embodiments, the transverse treatment planning component 112 can automatically suggest, and/or provide a visualization of, a recommended amount of upper arch expansion based on a static position of the lower arch. In some embodiments, the static position of the lower arch can be an existing position of lower arch, an intermediary position of the lower arch, or a final position of the lower arch following orthodontic treatment (e.g., aligners) and/or orthognathic surgery. The transverse treatment planning component 112 can identify one or more reference points within one or more 3D dental models of the patient. One of the 3D dental models can correspond to the lower jaw at the static position, and another one of the 3D dental models can correspond to the upper jaw. In some embodiments, the one or more 3D dental models can include multiple 3D models of the upper jaw, each one representing the upper jaw at a different treatment stage for a PE treatment plan. In some embodiments, each stage of the PE treatment plan can expand the palate by 0.25 millimeters, or by another amount (e.g., 0.1 mm, 0.3 mm, 0.5 mm). In some embodiments, different palatal expanders can expand the palate by different amounts. The reference points can correspond to predetermined landmarks, such as cusps on the upper and lower for molars. For example, a reference point can represent the midpoint between the cusps of the upper right first molar, and another reference point can represent the midpoint between the cusps of the lower right first molar. In some embodiments, a user can identify or define the one or more reference points, and/or can identify or define additional reference points. For example, the user can select which reference point(s) to use from a dropdown menu, and/or can identify a reference point by clicking on the relevant portion of the patient's teeth displayed in the UI. The transverse treatment planning component 112 can use the reference points to determine tooth measurements, which can be used to determine a recommended palatal expansion amount and/or a recommended palatal expansion treatment plan (including, for example, the number of palatal expanders in the recommended palatal expansion treatment plan).

In some embodiments, the transverse treatment planning component 112 can perform image processing on the 3D dental models to determine the location of the reference points. For example, the transverse treatment planning component 112 can implement feature detection, feature matching, and/or georeferencing to identify the coordinates in the 3D dental model corresponding to the reference points. In some embodiments, the transverse treatment planning component 112 can provide the 3D dental model(s) and the reference points as input to an AI model that is trained to provide indicators representing the locations of the reference points within the 3D dental models.

In some embodiments, the transverse treatment planning component 112 can implement one or multiple algorithms to determine the recommended amount of expansion. In some embodiments, the transverse treatment planning component 112 can add two to four millimeters to the recommended expansion amount, e.g., to avoid relapse.

In some embodiments, the transverse treatment planning component 112 can performing a staging of a simulated PE treatment plan, where each stage accounts for some set amount (e.g., 0.25 millimeters) of expansion. In some embodiments, the transverse treatment outcome component 114 can generate the simulated PE treatment plan. The simulated PE treatment plan can end at a maximum expansion amount (e.g., 12 millimeters of expansion), and/or a maximum number of treatment stages (e.g., 48 treatment stages). Each stage can be considered as a separate possible final position for the PE treatment plan. The transverse treatment planning component 112 can determine tooth measurements for each side of the mouth and for each stage, and can use the tooth measurements to determine a recommended palatal expansion amount.

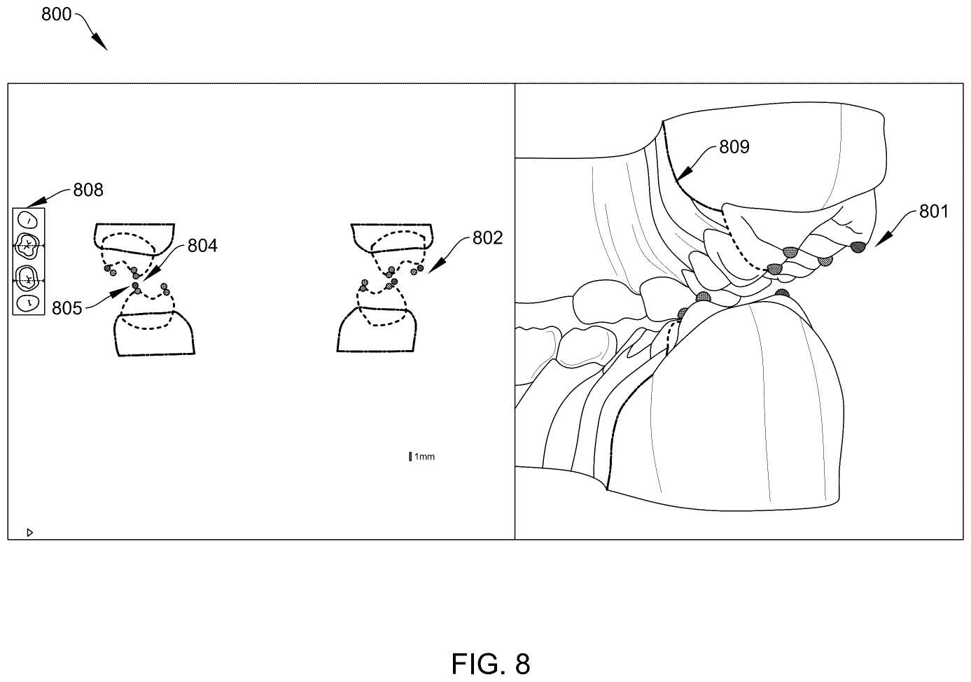

In some embodiments, to determine the tooth measurements, the transverse treatment planning component 112 can identify and/or generate a functional occlusal plane for the lower jaw. A functional occlusal plane can be described as an imaginary plane extending from the buccal cusps of premolars along the buccal cusps of the molars. The anterior teeth (from left canine to right canine of the respective jaw) may not be taken into account for the functional occlusal plane. The transverse treatment planning component 112 can build a second occlusal plane, extending in the mesial-distal direction from the tooth crown center of the first molar at the lower jaw. The transverse treatment planning component 112 can build a ray based on the intersection of the functional occlusal plane and the second occlusal plane. The transverse treatment planning component 112 can identify reference points. The reference points can represent midpoints for the tooth cusp at the first molar, including (a) the midpoint between the upper cusp lingual distal and the upper cusp lingula mesial, and (b) the midpoint between the lower cusp buccal distal and the lower cusps buccal mesial. The transverse treatment planning component 112 can project the midpoints (e.g., midpoints (a) and (b)) on to the ray, and can calculate the distance between the projections on the ray. This distance can represent a tooth measurement. The transverse treatment planning component 112 can then determine a tooth measurement (as described above) for each stage of the simulated PE treatment plan, and for each side (e.g., for the right side of the mouth and for the left side of the mouth). The transverse treatment planning component 112 can find the minimal value of all the tooth measurements determined for each side and for each stage of the simulated PE treatment plan, which will represent the cusps intersection stage. For example, the cusp intersection stage can represent the point at which the upper lingual molar cusps barely touch the lower buccal molar cusps, e.g., as illustrated in FIG. 8. It can be understood that if one side of the jaw has crossed (e.g., has reached the cusp intersection stage), it is not necessary to wait for the other side to reach the cusp intersection stage). Thus, recommended expansion amount and the recommended number of expanders can correspond to the stage of the minimal value of the tooth measurements.

In some embodiments, the transverse treatment planning component 112 can project the center of the upper and lower jaws onto the ray, and can determine a distance from the projection of the center of the jaws to the reference points (e.g., the midpoint between the upper cusp lingual distal and the upper cusp lingula mesial, and the midpoint between the lower cusp buccal distal and the lower cusps buccal mesial). The transverse treatment planning component 112 can determine a tooth measurement as the difference between the distances (e.g., upper_distance−lower_distance). The transverse treatment planning component 112 can determine this tooth measurement for every stage of the simulated PE treatment plan. When the difference is greater than zero, the upper jaw has moved further than the lower jaw, and thus the desired expansion has been reached. Thus, the transverse treatment planning component 112 can identify the recommended expansion amount and/or recommended number of expanders based on the stage at which the tooth measurement (e.g., the difference between the distances) is greater than zero.

In some embodiments, the tooth measurements can include (1) the difference between the midpoint between lingual cusps of the upper left first molar (UL) and the midpoint between the buccal cusps of the lower left first molar (LL), and (2) the difference between the lingual cusps of the upper right first molar (UR) and the midpoint between the buccal cusps of lower right first molar (LR). The transverse treatment planning component 112 can determine, for each stage in the simulated PE treatment plan, the minimum of modulus of the tooth measurement (e.g., m(stage)=min(|UL.x−LL.x|, |UR.x−LR.x|)). The transverse treatment planning component 112 can identify the recommended number of treatment stages as the stage where this value is the smallest (e.g., stageX=min(m(0), m(1), . . . , m(48))). The transverse treatment planning component 112 can determine the recommended expansion amount based on the recommended number of treatment stages and the expansion amount per stage (e.g., recommended expansion amount=stageX*0.25 millimeters). Other reference points and target measurements can also be used.

In some embodiments, the transverse treatment planning component 112 can store the reference points and tooth measurements used in determining the recommended PE treatment expansion and plan, e.g., in parameters data sore 142. In some embodiments, transverse treatment visualization component 116 can identify the reference points and/or tooth measurements (e.g., stored in parameters data store 142), and can include indicators of the reference points and/or tooth measurements in the 2D and/or 3D visualizations of the patient's teeth in the UI, as described herein. In some embodiments, the transverse treatment planning component 112 can store the recommended expansion amount and/or recommended number of expanders (e.g., in data stores 108). The transverse treatment visualization component 116 can provide the recommended expansion amount and/or recommended number of expanders to for display on a user device (e.g., computing device 160 and/or oral state capture system 110). In some embodiments, the transverse treatment visualization component 116 can enable a user (e.g., dental professional) to modify the recommended expansion amount and/or number of expanders (e.g., to add some overtreatment to avoid relapse). The transverse treatment planning component 112 can update the recommended expansion amount and/or number of expanders based on the user's modifications.

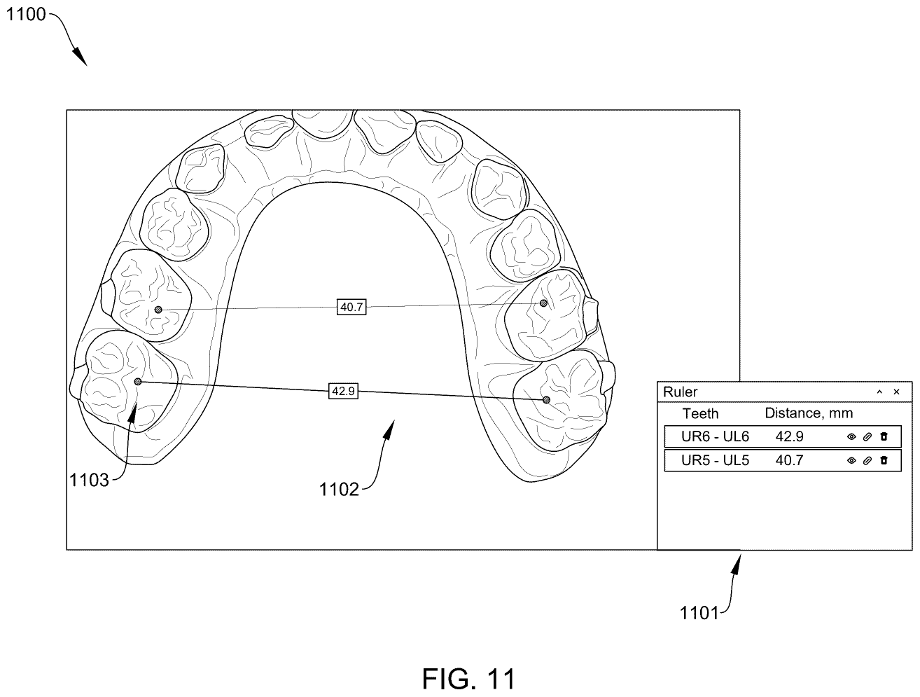

In some embodiments, the tooth measurements can include a trans-palatal width measurement (e.g., as described with respect to FIG. 11). For example, a typical maximally arch should have a trans-palatal width of 35 millimeters to 39 millimeters measured between the closest points of the upper first molars (e.g., the cementoenamel junction (CEJ) points). This trans-palatal width can accommodate a dentition of average size without crowding or spacing. In some embodiments, the tooth measurements can include the coronal projection measuring distance between jaw bones.

In some embodiments, the transverse treatment planning component 112 can access and/or implement an AI model to determine the recommended amount of expansion, by providing, as input to the AI model, the reference points and/or the tooth measurements. The AI model can provide, as output, the recommended palatal expansion amount.

In some embodiments, the transverse treatment outcome component 114 can provide a predicted outcome of a PE treatment. In some embodiments, the transverse treatment outcome component 114 can include and/or implement an AI model that is trained to predict the outcome of the particular patient's craniofacial structure. The craniofacial structure can include, for example, arch width, palate width, tooth positioning and/or placement, and/or palatal shape. In some embodiments, the AI model can be a forecasting model. In some embodiments, the transverse treatment outcome component 114 can include an AI model trained using a training dataset that includes historical palatal expander-treated cases. The training dataset can include upper arch/palatal 3D data before palatal expander treatment and after the palatal expander treatment. The training data set can include, for example, scan data of a patient before palatal expander treatment (including the arch width), planned expansion amount of the palatal expander treatment, scan data following the palatal expander treatment, the number of expanders worn during the palatal expander treatment, the actual expansion amount following the palatal expander treatment, and/or any other relevant information of the palatal expander treatment. Using the training dataset, the AI model can be trained to predict the outcome of the craniofacial structure of the patient caused by the expander treatment plan.

The transverse treatment outcome component 114 can provide, as input the trained AI model, the scan data of the patient (e.g., a 3D model of the patient's upper dental arch and/or lower dental arch, 2D projections of the 3D model of the patient's upper dental arch and/or lower dental arch, intraoral scans, etc.), as well as optionally the one or more parameter values associated with the patient and the patient's PE treatment plan. The AI model can provide, as output, a predicted outcome of the craniofacial structure of the patient caused by the patient's PE treatment plan. In some embodiments, the transverse treatment outcome component 114 can provide the recommended PE treatment plan, as provided by the AI model of transverse treatment planning component 112, as input to the second AI model, along with the scan data of the patient. The second AI model can then provide a predicted outcome of the craniofacial structure of the patient caused by the recommended PE treatment plan. In some embodiments, the predicted outcome can include the expansion amount caused by the PE treatment plan, the shape of the palate following the PE treatment plan, and/or the placement of the teeth following the PE treatment plan. In some embodiments, the predicted outcome data can be stored in outcomes data store 146. The predicted outcome may be determined for a PE treatment plan that has not yet been started, or for a PE treatment plan that is in an intermediate stage of treatment in embodiments. If the predicted outcome is for an intermediate stage of treatment, then additional information may be input into the AI model than is available for a treatment plan that is not yet started. For example, an amount of expansion that has occurred thus far may be provided to the transverse treatment outcome component 114 in addition to the other indicated possible inputs.

In some embodiments, the predicted outcome can include the outcome after the final expander in the PE treatment plan is worn and/or an outcome associated with one or more intermediate stages of treatment. In some embodiments, the predicted outcome can include the outcome of the patient's craniofacial structure a period of time after the final expander in the PE treatment, e.g., to show how the teeth are predicted to move after the PE treatment is completed. In some embodiments, the AI model can output a predicted outcome for a period of time after the PE treatment.

In some embodiments, the transverse treatment visualization component 116 can generate a 2D and/or a 3D visualization of the predicted outcome provided by the transverse treatment outcome component 114. The 3D visualization can display a 3D model of the patient's dentition and/or craniofacial structure, including the amount of expansion caused by the PE treatment plan, the shape of the palate following the PE treatment plan, and/or the placement of the teeth following the PE treatment plan. In some embodiments, the visualization can display the placement of upper teeth in relation the lower teeth, to allow a dental professional and/or a patient to visualize how the PE treatment plan will affect the overall dentition of the patient. In some embodiments, the PE treatment plan can include orthodontic treatment (e.g., including application of orthodontic aligners) following the PE treatment, and the 3D model can display the position of the teeth following the orthodontic treatment and/or at one or more intermediate stages of orthodontic treatment. The transverse treatment visualization component 116 can provide the user interface for the treatment planning system 115, which can be displayed on computing device(s) 160, in some embodiments. An example UI provided by transverse treatment visualization component 116 is described with respect to FIGS. 2, 3A, 3B, 7-11.

In some embodiments, the transverse treatment visualization component 116 can generate the 3D model of the patient's dentition and/or craniofacial structure using the data provided by the AI model of transverse treatment outcome component 114, and/or stored in outcomes data store 146. In some embodiments, the transverse treatment visualization component 116 can generate a 2D visualization of the patient's dentition and/or craniofacial structure. In some embodiments, the 2D visualization can be cross-section of the 3D model, e.g., passing through the first molars of the patient's dentition. The 2D cross-section view can provide a clear visualization of the relationship between the molars of the upper jaw and the molars of the lower jaw. A dental professional can use the 2D cross-section view to identify a desired expansion using the lower jaw as a reference, for example. In some embodiments, the transverse treatment visualization component 116 can provide access to UI controls that provide fine control over the position of the cross-section, e.g., in the mesial-distal direction. In some embodiments, the transverse treatment visualization component 116 can provide the 3D view and/or the 2D cross-section view for presentation in the UI of a user device (e.g., of computing device 160).

In some embodiments, the transverse treatment planning component 112 can enable a dental professional to plan and stage orthodontic treatment for the upper and/or lower dentition of the patient. That is, the transverse treatment planning component 112 can enable an orthodontic treatment plan for the upper and/or lower teeth, to be implemented in conjunction with and/or following the PE treatment plan. The orthodontic treatment plan can include prescribing aligners to align the upper and/or lower teeth with the predicted position of the upper teeth following the PE treatment plan. The transverse treatment visualization component 116 can display the position of the upper and/or lower teeth before, during, and after the orthodontic treatment plan.

In some embodiments, the transverse treatment outcome component 114 can determine a series of intermediate outcomes between the initial position and the final, predicted outcome of the PE treatment. The series of intermediate outcomes can correspond to intermediate stages of treatment, and to the expanders in the PE treatment plan associated with the respective intermediate stages of treatment. The transverse treatment visualization component 116 can generate a 3D model for each intermediate outcome, enabling a dental professional and/or patient to view the progress of the expansion expected to occur for the PE treatment plan and/or actual expansion experienced during execution of the PE treatment plan. In some embodiments, the intermediate positions can be determined using modeling software that simulates the pressure applied by each expander. For example, each expander can result in 0.25 millimeters of expansion of the palate, which may be modeled. In some embodiments, the intermediate positions can be determined using AI.

In some embodiments, the 3D model for each intermediate outcome can be displayed in the UI together a visualization of the dental appliance corresponding to the stage of treatment. In some embodiments, the transverse treatment visualization component 116 can provide, for display in the UI, the treatment plan and/or the stage of the treatment plan corresponding to the 3D model. In some embodiments, the transverse treatment visualization component 116 can provide, for display in the UI, a visualization of the dentition of the patient (e.g., the 3D model), and/or a visualization of a dental appliance worn over the visualization of the patient's dentition.

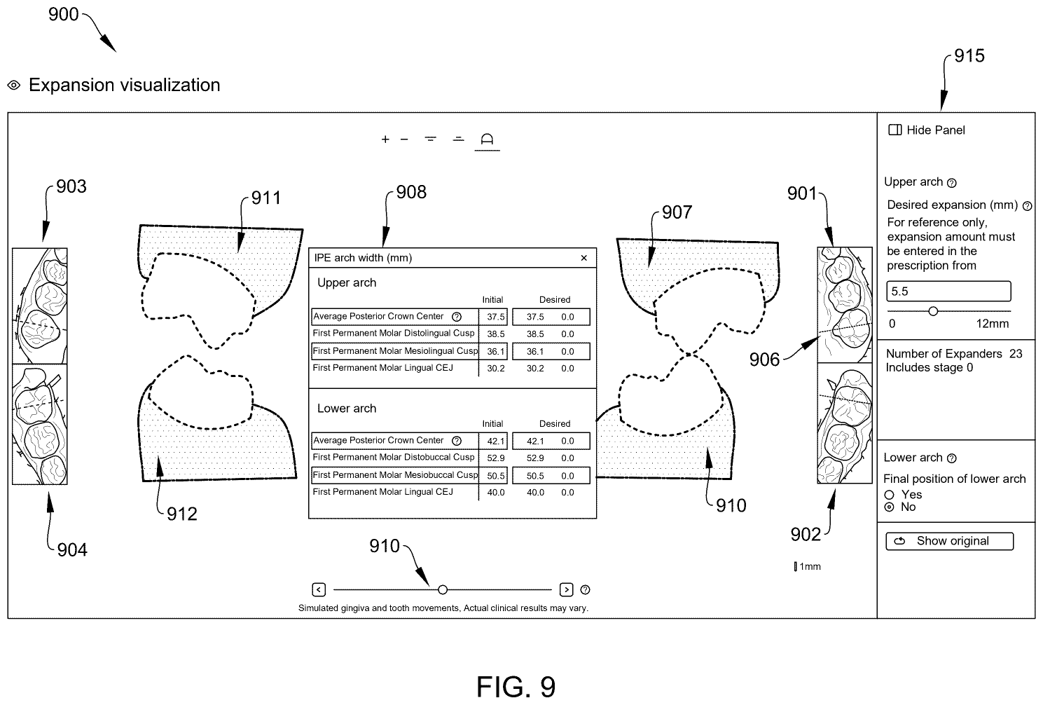

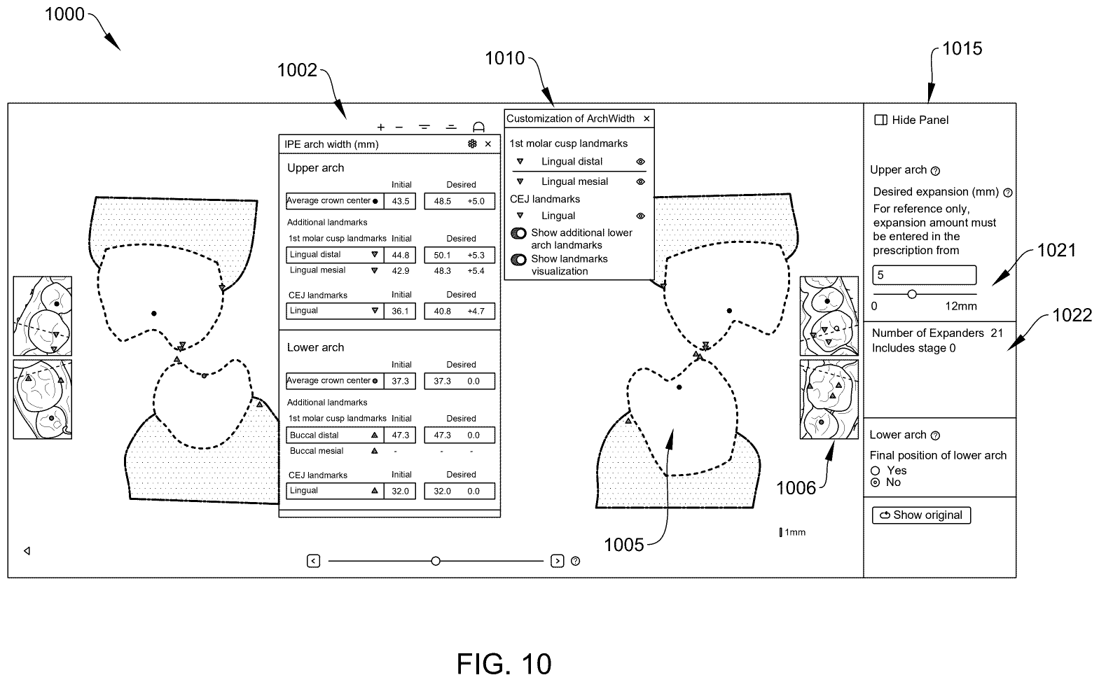

In some embodiments, the transverse treatment visualization component 116 can generate and/or provide visualizations of 2D cross-sectional planes and/or reference points in the UI. In some embodiments, the transverse treatment visualization component 116 can generate 2D cross-sections from the 3D dental models at specific locations. In some embodiments, the transverse treatment visualization component 116 can enable a user to identify the location of the 2D cross-sections, for example, by clicking on a location of tooth displayed in the UI and/or by using a sliding bar (e.g., as described with respect to FIG. 9). In some embodiments, the transverse treatment visualization component 116 can identify real images of the patient's teeth (e.g., corresponding to one or more photographs or scans of the patient's teeth stored in scan data store 144), and provide the images for display in the UI. The images can represent an occlusal view of the patient's teeth (or a subset of the patient's teeth, e.g., displaying the molars). The transverse treatment visualization component 116 can add lines over the images to indicate the specific locations at which the 2D cross-sectional planes intersect the teeth. In some embodiments, the transverse treatment visualization component 116 can provide visualizations of both jaws and both sies of the mouth simultaneously. In some embodiments, the transverse treatment visualization component 116 can identify reference points (e.g., as stored in data store 142 and/or as identified by transverse treatment planning component 112), and can include visualizations of the identified reference points on the UI (e.g., as described with respect to FIG. 10). In some embodiments, the transverse treatment visualization component 116 can provide a ruler tool to quantify palatal expansions at various stages of treatment, and can display the measurements alongside the visualizations (e.g., as described with respect to FIGS. 9-11).

Applicant hereby incorporates by reference the following application as if set forth fully here, as an example of palatal expansion treatments: application Ser. No. 18/982,401, filed on Dec. 16, 2024.

Applicant hereby incorporates by reference the following patent as if set forth fully here, as an example of method for making and using palatal expanders: U.S. Pat. No. 11,273,011B2, issued on Mar. 15, 2022.

FIG. 2 depicts an example user interface (UI) 200 of a PE previewer and treatment manager 202, in accordance with some embodiments of the present disclosure. In some embodiments, UI 200 (or portions of UI 200) can be displayed on a computing device of a patient and/or a doctor (e.g., corresponding to computing device 160 of FIG. 1), and/or on a scanning device (e.g., of oral state capture system(s) 110 of FIG. 1). The PE previewer and treatment manager 202 can include the patient ID 214, as well as a series of tools and options available to a dental professional. The UI 200 can include a three dimensional view of the patient's dentition 270 (e.g., as generated by transverse treatment visualization component 116 of FIG. 1). The three dimensional view can display the predicted outcome of the patient's dentition as a result of a particular palatal expansion treatment plan (e.g., as provided by transverse treatment outcome component 114). The UI 200 can enable a dental professional to modify the palatal expansion treatment plan, e.g., by modifying the expansion amount 260 and/or modifying the parameters of the treatment plan. In some embodiments, the dental professional can modify the parameters, such as the expander width 261 (e.g., the vertical clearance of the expander, sometimes referred to as the thickness of the expander), the placement of the attachments on the teeth 262, which teeth are covered by the expander (expander coverage 263), which part of the palate is covered by the expander (palatal coverage 264), and/or the amount of force applied by each expander 265 via the UI 200. In some embodiments, the UI 200 can enable a dental professional to modify the amount of expansion, e.g., using the expansion slider 260. The expansion slider 260 can be an interactive tool.

Changes to one or more of the parameters 260-265 can generate changes to the upper jaw displayed in the 3D visualization of the patient's dentition (e.g., to the predicted final positions of teeth of the upper jaw after PE treatment based on the chosen parameters). In some embodiments, the change(s) to the parameter(s) can be sent to transverse treatment outcome component 114, which can input the change(s) to the parameter(s) to the AI model of transverse treatment outcome component 114 to receive an updated predicted outcome. The transverse treatment visualization component 116 can generate an updated 3D visualization 270 to display the predicted outcome corresponding to the modified parameter(s).

In some embodiments, the PE previewer and treatment manager 202 can include an expansion recommendation 274. The expansion recommendation 274 can provide a recommended PE treatment plan for the particular patient (e.g., as provided by transverse treatment planning component 112). In some embodiments, the clinical analysis 220 can enable a dental professional to set certain treatment preferences. The treatment preferences can affect the expansion recommendation 274. That is, the recommended PE treatment plan can be personalized to the dental professional's preferences. The preferences can include, for example, an indication of whether the dental professional prefers a moderate amount of expansion, a small amount of expansion, or a greater amount of expansion (e.g., as compared to the average dental professional). In some embodiments, the clinical analysis tool 220 can analyze prior PE treatments carried out by the dental professional, and can provide the indication of whether the dental professional prefers moderate, smaller, or greater expansion. In some embodiments, the clinical analysis tool 220 can allow the dental professional to limit the analysis of prior PE treatments to a certain timeframe, a certain patient age group, a certain expansion amount range, and/or any other applicable analysis filter. Thus, the expansion recommendation 274 can be customized to a dental professional's preferences. In some embodiments, the 3D visualization 270 can display the recommended expansion, as provided by the expansion recommendation tool 274.

In some embodiments, the movement of the teeth that are not directly moved by the series of expanders can be simulated, and displayed in a differentiated manner (e.g., in a different color, or partially transparent). For example, as illustrated in FIG. 2, the placement of tooth 272 is not directly affected by the PE treatment plan, and thus is displayed partially transparent. In some embodiments, the teeth that are not directly moved by the expanders can be hidden.

In some embodiments, the vertical positioning of the upper jaw to the lower jaw in the 3D visualization 270 of the patient's dentition can display a disocclusion. The amount of disocclusion can be random (e.g., within a specified range), or can be based on the width of one of the expanders in the series of expanders. The amount of disocclusion based on the width of one of the expanders can simulate the amount of clinical disocclusion during the treatment.

In some embodiments, the UI 200 can include a set of measurement tools 210, including a grid 212, a ruler 214, the intercuspal (IC) jaw width measurement 216, and the buccal overjet (OJ) measurement 218. The grid 212 can toggle a visual grid to aid the dental professional to view the teeth placement in relation to each other (e.g., how the top teeth align with the bottom teeth). The ruler 214 can enable a dental professional to measure distances in the visualization, e.g., to measure the distance between two teeth. The IC jaw width measurement 216 can provide a measurement of the distance between the second molars. The buccal overjet 218 can provide a measurement of the horizontal overlap of the upper teeth over the lower teeth on the cheek (buccal) side when the jaw is closed.

In some embodiments, the UI 200 can include an optional lower jaw adjustment 222 tool, which can enable the lower visualization 271, allowing a dental professional to view the 3D visualization of the patient's lower detention at the initial stage or simulated post treatment or intermediate treatment. That is, in some embodiments, the UI 200 can enable the dental professional to visualize the current position of the lower jaw, as well as a simulation representing a particular orthodontic treatment, such as alignment (e.g., clear aligner treatment used on the teeth of the lower jaw). Thus, the simulated post treatment or intermediate option can enable a dental professional to incorporate, in the PE previewer and treatment manager 202, an orthodontic treatment of the lower teeth. The orthodontic treatment of the lower teeth can include aligners to align the lower teeth with the position of the upper teeth as a result of the palatal expansion treatment plan. The PE previewer and treatment manager 202 can enable a dental professional to view a 3D visualization of the patient's dentition (including both upper and lower teeth) representing the predicted outcome of the PE treatment plan as well as the post-orthodontic treatment plan, e.g., by selecting the “simulated post treatment” option. That is, the 3D visualization can change to reflect either the initial stage of the lower teeth, or the simulated post treatment outcome (for an intermediate treatment stage or final treatment stage) of the tooth positioning and placement during or post orthodontic treatment of the lower teeth. In some embodiments, the UI can enable a dental professional to visualize the predicted tooth positioning and/or placement at each stage of the PE treatment plan and/or of the orthodontic treatment plan. In some embodiments, the PE treatment plan can include orthodontic treatment following the palatal expansion, in order to position the teeth to meet a certain goal.

In some embodiments, the PE previewer and treatment manager 202 can enable a user to manipulate the display and/or orientation of the 3D visualization. For example, the zoom 224, rotate 226, and/or pan 228 elements (e.g., buttons, sliders, etc.) can enable a user to manipulate the display of the 3D visualization. In some embodiments, the PE previewer and treatment manager 202 can enable a user to display certain portions of the 3D visualization of the patient's' dental model. For example, visual elements for upper 230, maxilla 323, left 234, anterior 236, right 238, and/or mandible 240 can enable a user to select one or more regions of the dental model to view in section 270.

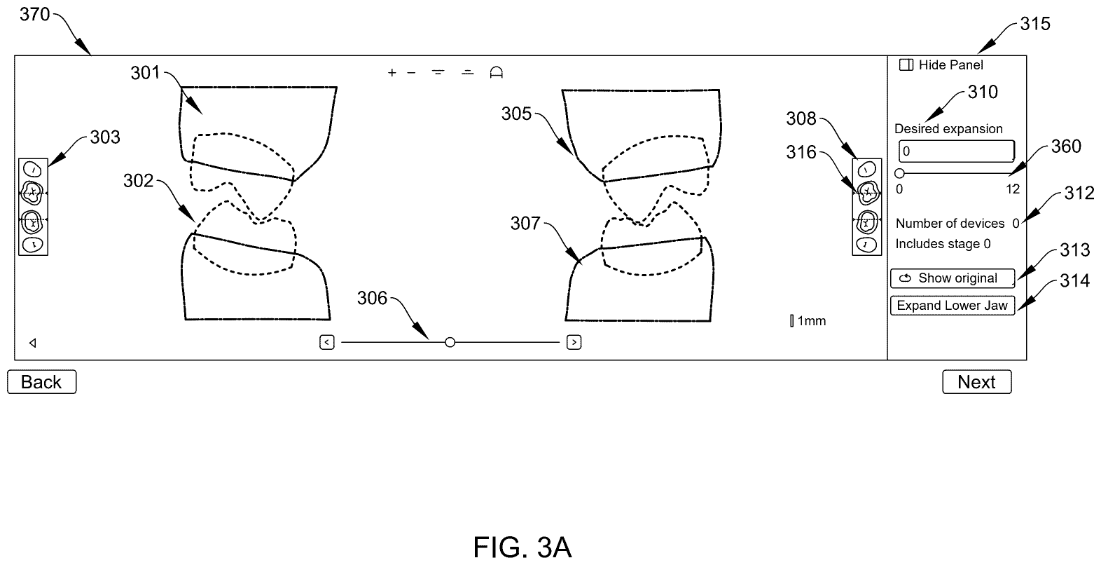

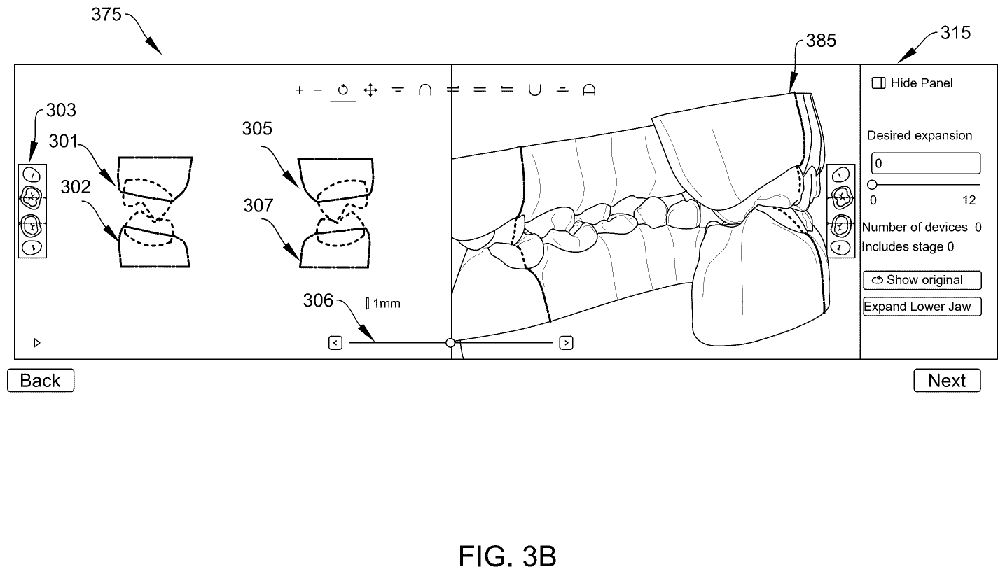

In some embodiments, the PE previewer and treatment manager 202 can enable a user to view the patient's dentition in a 3D view (e.g., as illustrated in FIG. 2 as UI portion 270), in a 2D view (e.g., a 2D cross sectional view as illustrated in FIG. 3A as UI portion 370), or in a combination of 3D and 2D view (e.g., as illustrated in FIG. 3B as UI portion 375). For example, the UI can include a 3D/2D view toggle (not pictured), and/or a tool to select 2D view, 3D view, or a combination of 3D and 2D. In some embodiments, the 2D view can be a cross-section of the 3D view. In some embodiments, the PE previewer and treatment manager 202 can enable the user to identify the position of the cross-section. For example, the UI can include a control that provides the user with fine control over the position of the cross-section, e.g., in the mesial-distal direction. In some embodiments, the 2D cross-section can pass through the first molar. In the 2D view, the relationship between the molars and the opposite jaw can be visually clear, thus enabling the dental professional to identify the desired expansion using the law jaw as a reference. In some embodiments, the 3D view and the 2D view can be displayed side-by-side, in order to provide the dental professional with the most information on the intended treatment plan.

In some embodiments, the UI 200 can display an AI generated simulation of the evolution of the palate shape over the course of the PE treatment plan.

FIG. 3A depicts a portion 370 of an example UI of a PE previewer and treatment management tool displaying a two dimensional view, in accordance with some embodiments of the present disclosure. In some embodiments, UI 370 (or portions of UI 370) can be displayed on a computing device of a patient and/or a doctor (e.g., corresponding to computing device 160 of FIG. 1), and/or on a scanning device (e.g., of oral state capture system(s) 110 of FIG. 1). In some embodiments, the UI can correspond to UI 200 of FIG. 2, and the portion 370 can correspond to portion 270 of FIG. 2. The 2D cross-section displayed in the UI portion 370 can display the a cross-section of the 3D model (e.g., of the 3D model displayed in 270 of FIG. 2) passing through the first molars, for example. The 2D cross-section displayed in UI portion 370 can display the position of the upper and lower teeth as a result of a particular palatal expander treatment plan (e.g., as provided by transverse treatment outcome component 114). For example, UI portion 370 can display the 2D cross-section of the upper left first molar 301, the 2D cross-section of the lower left first molar 302, the 2D cross-section of the upper right first molar 305, and the 2D cross-section of the lower right first molar 307. The side panels 303 and 308 can display dotted lines that depict where the cross-section intersects the teeth to generate the 2D cross-sectional view displayed in UI portion 370. For example, dotted line 316 depicts where the cross-section intersects the upper right first molar depicted in 308 to generate the 2D cross-sectional view 305. The sliding bar 306 can enable a user (e.g., a dental professional) to change the position of cross-section along the teeth.

The UI portion 370 can also include, in some embodiments, a panel 315 that includes additional features. Panel 315 can include a slider tool 360 and/or a desired expansion text box 310 that enables the user (e.g., a dental professional) to change the desired expansion amount (e.g., from between 0 millimeters to 12 millimeters). As the user changes the desired expansion amount (e.g., by entering an expansion amount in the desired expansion textbox 310 and/or by sliding the slider tool 360 to a new desired expansion amount value), the UI portion 370 can automatically change the position of the teeth to show the relationship between the upper teeth and the lower teeth during and/or after the PE treatment plan. For example, to view the outcome during the PE treatment plan, the dental professional can slide the slider tool 360 to an amount less than the total expansion amount. In some embodiments, the panel 315 of UI portion 370 can include the number of devices recommended to achieve the desired expansion amount, illustrated as number of devices 312. In some embodiments, the panel 315 of UI portion 370 can also include a “show original” button 313, which when selected by a user, can revert the 2D cross-section displayed in the UI portion 370 to the original version, e.g., before the dental professional made modifications to the treatment plan (e.g., before the dental professional modified the desired expansion amount). In some embodiments, panel 315 can include a “expand lower jaw” button 314, which can enable a user (e.g., a dental professional) to visualize the final position of the lower dentition (e.g., first molars 302 and 307) following an ongoing or planned treatment, e.g., an orthodontic treatment plan. For example, when a user clicks on the “expand lower jaw” button 314, the visualization of the lower dentition (e.g., first molars 302 and 307) can be updated to reflect a final position of an ongoing or planned orthodontic treatment.

FIG. 3B depicts a portion 375 of an example user interface of a PE previewer and treatment management tool displaying a two dimensional view and a three dimensional view, in accordance with some embodiments of the present disclosure. In some embodiments, UI 375 (or portions of UI 375) can be displayed on a computing device of a patient and/or a doctor (e.g., corresponding to computing device 160 of FIG. 1), and/or on a scanning device (e.g., of oral state capture system(s) 110 of FIG. 1). In some embodiments, the UI can correspond to UI 200 of FIG. 2, and the portions 375 and/or 385 can correspond to portion 270 of FIG. 2. As illustrated in FIG. 3B, the left-hand-side of the UI can depict the 2D cross-section of the 3D model, and the right-hand-side of the UI can depict the 3D model. The UI portion 375 can include a control 375 to enable the user to select the position of the cross-section, e.g., in the mesial-distal direction. In some embodiments, UI portion 375 can include similar features as UI 370, including, for example, panel 315, 2D cross-sectional view of the upper left first molar 301, lower left first molar 302, upper right first molar 305, and lower right first molar 307, side panels 303 and 308, and sliding bar 306.

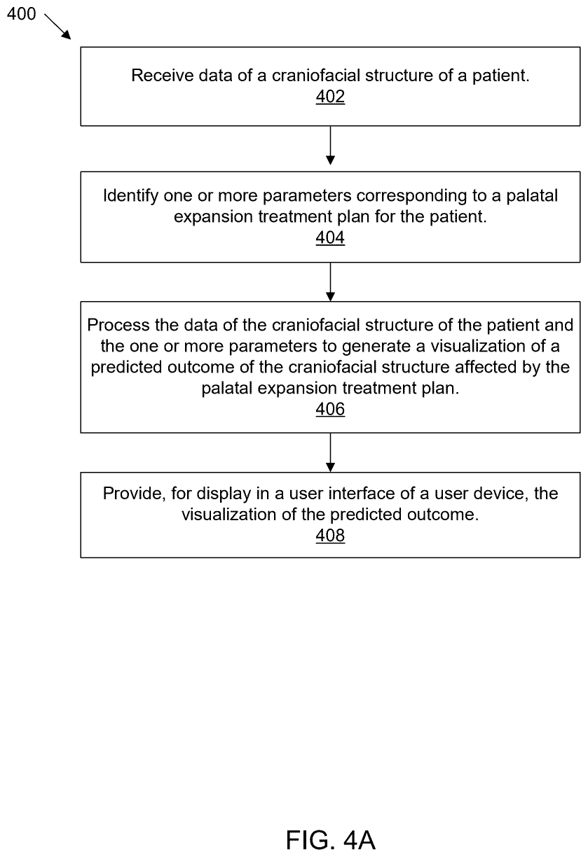

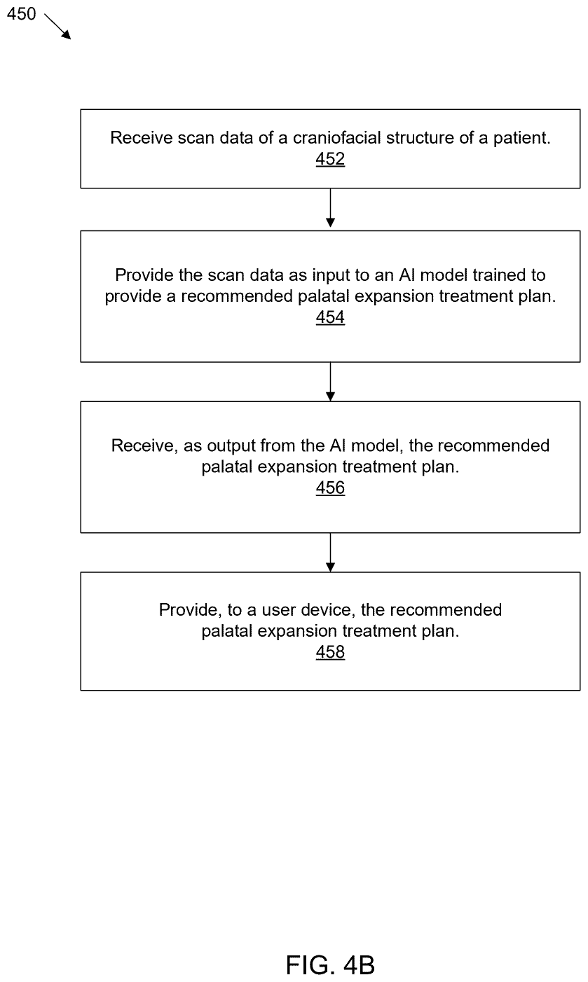

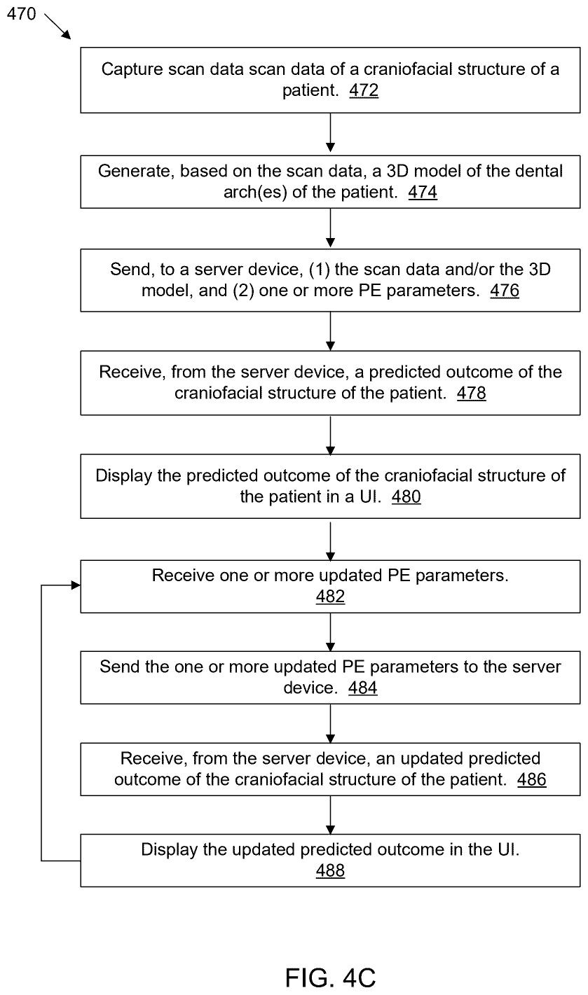

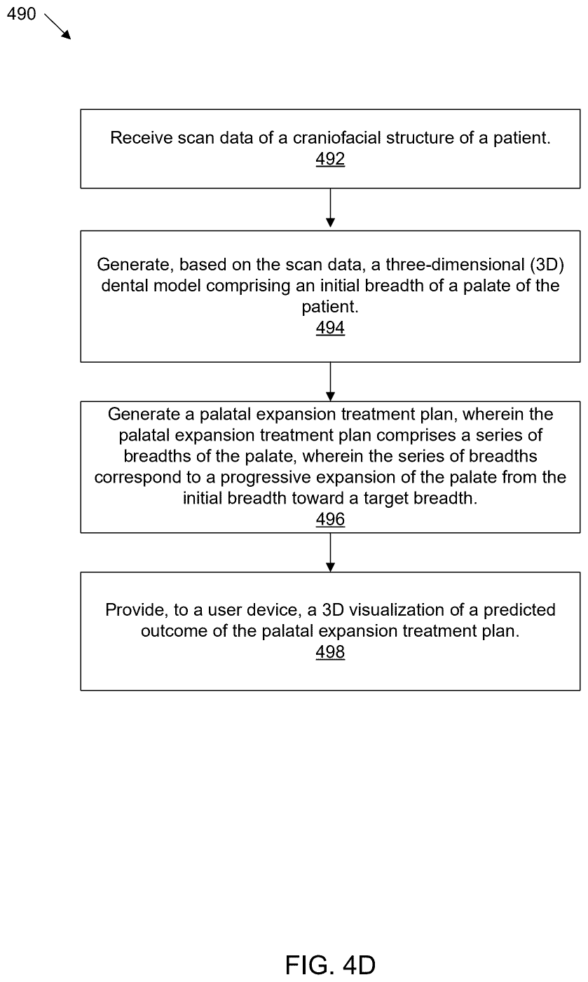

FIGS. 4A-D illustrates flow diagram of example method 400, 450, 470, 490 for implementing a PE previewer and treatment management tool, in accordance with some embodiments of the present disclosure. Method 400, 450, 470, and/or 490 may be performed by a processing device that may include hardware, software, or a combination of both. The processing device may include one or more central processing units (CPUs), graphics processing units (GPUs), field-programmable gate arrays (FPGAs), application-specific integrated circuits (ASICs), or the like, or any combination thereof. In one embodiment, method 400, 450, 470, and/or 490 may be performed by the processing devices and the associated algorithms, e.g., as described in conjunction with FIG. 1. In embodiments, method 400, 450, 470, and/or 490 is performed by processing logic comprising hardware, software, firmware, or a combination thereof. In certain embodiments, method 400, 450, 470, and/or 490 may be performed by a single processing thread. Alternatively, method 400, 450, 470, and/or 490 may be performed by two or more processing threads, each thread executing one or more individual functions, routines, subroutines, or operations of the method. In an illustrative example, the processing threads implementing method 400, 450, 470, and/or 490 may be synchronized (e.g., using semaphores, critical sections, and/or other thread synchronization mechanisms). Alternatively, the processing threads implementing method 400, 450, 470, and/or 490 may be executed asynchronously with respect to each other. Therefore, while FIGS. 4A, 4B, 4C, 4D and the associated descriptions list the operations of method 400, 450, 470, and/or 490 in a certain order, in some embodiments, at least some of the described operations may be performed in parallel and/or in a different order. In some embodiments, one or more operations of method 400, 450, 470, and/or 490 is not performed.

FIG. 4A illustrates a flow diagram of an example method 400 for providing a visualization of a predicted the outcome of a palatal expansion treatment plan, in accordance with some embodiments of the present disclosure.

At block 402, processing logic can receive data of a craniofacial structure of a patient. The craniofacial structure can include the bones of the skull and/or face, including the maxilla (e.g., the upper jawbone). In some embodiments, the data corresponds to scan data, e.g., stored as part of scan data store 144 of FIG. 1. In some embodiments, the data of the craniofacial structure of the patient can include a 3D dental model.

At block 404, processing logic can identify one or more parameters corresponding to a palatal expansion treatment plan for the patient. In some embodiments, the palatal expansion treatment plan can include multiple incremental palatal expanders used to cause movement in one or more parts of the craniofacial structure of the patient. In some embodiments, the palatal expansion treatment plan can include one expander to cause movement in one or more parts of the craniofacial structure of the patient. The parameters can include, for example, an amount of expansion, a vertical clearance measurement of at least one expander of the plurality of expanders, a placement of an attachment on a tooth of a dentition of the patient, a first identification of at least one tooth covered by the at least one expander of the plurality of expanders, a second identification of a corresponding part of a palate of the patient covered by the at least one expander of the plurality of expanders, or a second amount of transverse force applied by the at least one expander of the plurality of expanders.

In some embodiments, processing logic can receive at least a subset of the one or more parameters from a user device (e.g., computing device 160 of FIG. 1). For example, a user of the user device (e.g., a dental professional using computing device 160) can input at least some of the parameter(s). In some embodiments, processing logic can receive at least a subset of the one or more parameters as output from an AI model (e.g., of transverse treatment planning component 112). For example, processing logic can provide the scan data as input to an AI model, and processing logic can receive, as output from the AI model, a recommended PE treatment plan that includes treatment plan parameters (e.g., as further described with respect to FIG. 4B).

At block 406, processing logic can process the data of the craniofacial structure of the patient and the one or more parameters to generate a visualization of a predicted outcome of the craniofacial structure affected by the palatal expansion treatment plan.

In some embodiments, processing logic can provide the data and the one or more parameters to an AI model that is trained to provide a predicted outcome of the patient's craniofacial structure caused by the palatal expansion treatment plan (e.g., as described with respect to transverse treatment outcome component 114). Processing logic can receive, as output from the AI model, the predicted outcome of the craniofacial structured caused by the palatal expansion treatment plan. The predicted outcome can include, for example, an amount of expansion of a palate of the patient, a predicted placement of at least one tooth in a dentition of the patient, and/or a predicted shape of the palate of the patient. Processing logic can generate a visualization of the predicted outcome of the craniofacial structure. In some embodiments, the visualization can include a 3D visualization of the predicted outcome of the craniofacial structure of the patient.

At block 408, processing logic provides, for display in a user interface of a user device (e.g., computing device 160, and/or a device of oral state captures system(s) 110 of FIG. 1), the visualization of the predicted outcome. Example UIs are described with respect to FIGS. 2, 3A-B. In some embodiments, the user device can correspond to a patient's device, a doctor's device, and/or a scanning device.

In some embodiments, processing logic receives a modification to one of the one or more parameters corresponding to the palatal expansion treatment plan. Processing logic can provide the modified parameter(s) as additional input to the AI model. Processing logic can receive an updated predicted outcome of the patient's craniofacial structure corresponding to the modified parameter(s). Processing logic can update the visualization to reflect the updated predicted outcome, and can provide the updated visualization for display in the UI of the user interface.

In some embodiments, processing logic can generate, based on the processing of the data and the one or more parameters, a 3D dental model of the patient affected by the palatal expansion treatment plan. Processing logic can generate a two-dimensional cross-section view of the 3D dental model, and can provide, for display in the UI of the user device, the two-dimensional cross-section view (e.g., as displayed in FIG. 3A). In some embodiments, the 2D cross-section view can be displayed next to the 3D model (e.g., as displayed in FIG. 3B). In some embodiments, the 2D cross-section view can replace the 3D model view in the UI.

In some embodiments, processing logic can identify a position for the cross-section in the mesial-distal direction of the craniofacial structure of the patient, and can generate the 2D cross-section at the identified position. In some embodiments, the position can be provided as input from a user device (e.g., a dental professional can identify the desired position of the 2D cross-section view, as depicted as tool 385 in FIG. 3B).

FIG. 4B illustrates an flow diagram of an example method 450 for providing a providing a recommended palatal expansion treatment plan, in accordance with some embodiments of the present disclosure. At block 452, processing logic receives scan data of a craniofacial structure of a patient. In some embodiments, the scan data can correspond to data stored in the scan data store 144 of FIG. 1.

At block 454, processing logic provides the scan data as input to an AI model that is trained to provide a recommended palatal expansion treatment plan for the patient. In some embodiments, processing logic identified one or more values corresponding to palatal expansion treatment plan parameters. These palatal expansion treatment plan parameters can reflect preferences of the dental professional, for example (e.g., as descried with respect to clinical analysis 220 of FIG. 2). The one or more values can be provided as additional input to the AI model.

At block 456, processing logic receives, as output from the AI model, the recommended palatal expansion treatment plan. In some embodiments, the recommended palatal expansion treatment plan include an amount of expansion of a palate of the patient and/or a number of expanders in the recommended palatal expansion treatment plan. In some embodiments, the recommended palatal expansion treatment plan comprises a series of palate breadths corresponding to a progressive expansion of a palate of the patient from an initial breadth toward a target breadth. In some embodiments, the recommended palatal expansion treatment plan comprises data for one or more dental appliances, which may be worn by a patient in sequential order to implement the recommended palatal expansion treatment plan. In some embodiments, the one or more dental appliances comprises a single palatal expander that can be adjusted to different widths to perform palatal expansion. In some embodiments, the one or more dental appliances include a sequence of palatal expanders. Examples of palatal expanders that may be used include a Hyrax appliance (a fixed appliance with bands that wrap around the molars, and that includes a screw that can be tightened with a key), a rapid palatal expander (a rigid appliance with metal bands or rings that attached to the upper molars, and that includes a screw that can be tightened with a key), a Biedermann appliance, a surgically assisted rapid palatal expander (an expander that is placed in the mid-palatal suture by an oral surgeon to perform palatal expansion), a quad helix (a fixed appliance that is attached to the back molars, and that expands over time without manual adjustments), adjustable removable palatal expanders (clear retainer-like devices with a center screw that can be adjusted for small amounts of expansion), and non-adjustable removable palatal expanders (e.g., incremental palatal expanders designed to be used in sequence). In some embodiments, the one or more dental appliances comprises one or more incremental palatal expanders. In some embodiments, the one or more dental appliances comprise one or more 3D printed incremental palatal expanders. In some embodiments, processing logic can provide instructions (e.g., to the user device) to fabricate a series of palatal expanders corresponding to the palatal expansion treatment plan. In some embodiments, the dental appliances can be fabricated by fabrication machine(s) 170 of FIG. 1.

At block 458, processing logic can provide, to a user device (e.g., user device 160, and/or a device of oral state captures system(s) 110 of FIG. 1), the recommended palatal expansion treatment plan.

In some embodiments, processing logic can provide, as input to a second AI model, the scan data of the craniofacial structure of the patient and the recommended palatal expansion treatment plan. The second AI model can be trained to provide a predicted outcome of the craniofacial structure of the patient. Processing logic can receive, as output from the second AI model, the predicted outcome of the craniofacial structure of the patient. Processing logic can generate a visualization of the predicted outcome of the craniofacial structure, and can provide, for display in a user interface of the user device, the visualization of the predicted outcome. In some embodiments, the visualization can include a 3D visualization and/or a 2D visualization. Example UIs are described with respect to FIGS. 2, 3A-B.

In some embodiments, processing logic can identify one or more parameters corresponding to the recommended palatal expansion treatment plan, and can provide the one or more parameters as additional input to the second AI model. In some embodiments, processing logic can receive modifications to the one or more parameters, and can provide the modified parameters as additional input to the second AI model. Processing logic can receive an updated predicted outcome from the second AI model, and can generate an updated visualization of the updated predicted outcome. The processing logic can provide the updated visualization for display in the UI of the user device.

FIG. 4C illustrates a workflow diagram of an example method 470 for presenting a predicted outcome in a UI, in accordance with some embodiments of the present disclosure. In some embodiments, a client device (e.g., computing device 160, and/or a device of oral state captures system(s) 110 of FIG. 1) can perform method 470. As an illustrative example, method 470 can be performed at a doctor's client device, in which the output of the treatment planning system 115 is received and presented in a user interface.

At block 472, processing logic can capture scan data of a craniofacial structure of a patient. In some embodiments, the scan data can correspond to scan data store 144 of FIG. 1. As an illustrative example, a dental professional can perform a scan of the patient, e.g., using oral state capture system 110 of FIG. 1. The scan can be an intraoral scan, e.g., performed using an intraoral scanner. At block 474, processing logic can generate, based on the scan data, a 3D model of dental arch(es) of the patient, e.g., as described throughout.

At block 476, processing logic can send, to a server device (e.g., computing device 105 of FIG. 1), the scan data and/or the generated 3D model, along with one or more PE parameters of a PE treatment plan. In some embodiments, the PE parameters can correspond to the dental professional. For example, in some embodiments, the parameters data store 142 of FIG. 1 can store one or more parameters that correspond to the particular dental professional, such as the dental professional's treatment style or preferences, a history of the dental professional's treatments (including the plan and/or the outcomes), and so on. In some embodiments, the dental professional can provide one or more PE parameters, e.g., via a user interface. For example, the UI of device (e.g., computing device 160, and/or a device of oral state captures system(s) 110 of FIG. 1) can enable the device user (e.g., the dental professional) to provide one or more PE parameters (e.g., expander width, placement of the attachments on the teeth, palatal coverage, amount of force applied by each expander, number of expanders, the amount of palatal expansion, etc.). In some embodiments, the PE parameters can correspond to the patient (e.g., patient age). In some embodiments, the one or more parameters can be automatically determined from data stored in data store(s) 108 and/or can be received from input provided by the user of the device. In some embodiments, processing logic can store and/or access the PE parameter(s) in parameter data store 142.

At block 478, processing logic can receive a predicted outcome of the craniofacial structure of the patient caused by the palatal expansion treatment plan of the one or more parameters. In some embodiments, the predicted outcome (and/or data associated with the predicted outcome) can be stored in outcomes data store 146 of FIG. 1. In some embodiments, processing logic can receive an indication that the predicted outcome has been generated and/or stored in outcomes data store 146, and processing logic can access the predicted outcome data stored in outcomes data store 146. In some embodiments, processing logic can receive (and/or identify) data corresponding to the predicted outcome, and can generate a 3D model based on the data corresponding to the predicted outcome.

At block 480, processing logic can display the predicted outcome of the craniofacial structure of the patient in a UI of the device (e.g., as described with respect to FIGS. 2, 3A-B).

In some embodiments, at block 482, processing logic can receive one or more updated PE parameters. In some embodiments, a user of the device (e.g., a dental professional) can modify one or more of the parameters, and/or can provide additional parameter values. As an illustrative example, the user can modify the expansion amount parameter, e.g., using the slide bar 260 of FIG. 2. The new expansion amount can be an updated PE parameter. The user can modify any or all of the PE parameters. In some embodiments, the updated PE parameters can correspond to the patient and/or the dental professional. In some embodiments, processing logic can store and/or access the updated parameter(s) in parameter data store 142.

At block 484, processing logic sends the one or more updated PE parameters to the server device (e.g., computing device 105 of FIG. 1). The server device can provide an updated predicted outcome of the craniofacial structure of the patient based on the updated PE parameter(s). At block 486, processing logic can receive the updated predicted outcome of the craniofacial structure of the patient. In some embodiments, the updated predicted outcome (and/or data associated with the updated predicted outcome) can be stored in outcomes data store 146 of FIG. 1. In some embodiments, processing logic can receive an indication that the updated predicted outcome has been generated and/or stored in outcomes data store 146, and processing logic can access the updated predicted outcome data stored in outcomes data store 146. In some embodiments, processing logic can receive (and/or identify) data corresponding to the updated predicted outcome, and can generate a 3D model based on the data corresponding to the updated predicted outcome. At block 488, processing logic can display the updated predicted outcome in the UI (e.g., as described with respect to FIGS. 2, 3A-B). In some embodiments, blocks 482-488 can be repeated, e.g., as updated parameter(s) become available.

FIG. 4D illustrates a flow diagram of an example method 490 for providing a three-dimensional visualization of a predicted outcome of a palatal expansion treatment plan, in accordance with some embodiments of the present disclosure.

At block 492, processing logic receives scan data of a craniofacial structure of a patient. In some embodiments, the scan data can correspond to data stored in the scan data store 144 of FIG. 1. In some embodiments, scan data can include cone-beam computed tomography data, thus enabling soft tissue morphing.

At block 494, processing logic generates, based on the scan data, a three-dimensional (3D) dental model comprising an initial breadth of a palate of the patient.

At block 496, processing logic generates a palatal expansion treatment plan that includes a series of breadths of the palate. The series of breadths can correspond to a progressive expansion of the palate of the patient, from the initial breadth toward a target breadth. In embodiments, processing logic generates the palatal expansion treatment plan based on processing of a 3D model of a patient's current upper dental arch and/or lower dental arch, images (e.g., scan data) of the upper and/or lower dental arch, projections of a 3D model of the upper and/or lower dental arch, etc. using a trained AI model and/or a rules-based system. The trained AI model and/or rules-based system may process the input and output a recommended target palate width and/or a series of intermediate palate widths, each of which may be associated with a different stage of treatment and/or a different palatal expander.

At block 498, processing logic provides, to a user device (e.g., computing device 160, and/or a scanning device that is part of oral state captures system(s) 110 of FIG. 1), a 3D visualization of a predicted outcome of the palatal expansion treatment plan. In some embodiments, the 3D visualization can be provided for display in a user interface of a scanning device, a patient's computing device, and/or a doctor's computing device.

In some embodiments, processing logic can provide, the user device, the palatal expansion treatment plan. In some embodiments, the palatal expansion treatment plan can include one or more treatment stages. Each treatment stage can be associated with one or more dental appliances that are usable to implement the palatal expansion treatment plan. For example, each treatment stage can include instructions for the patient to wear a dental appliance (e.g., an expander), or one or more incremental palatal expanders in a series of incremental palatal expanders. In some embodiments, a visual representation of a first dental appliance of the one or more dental appliances can be provided for display in a user interface of the user device. The first dental appliance can correspond to a treatment stage of the one or more treatment stages. In some embodiments, the treatment stage can be displayed in a first portion of the UI, and the first dental appliance can be displayed in a second portion of the UI. In some embodiments, a visualization of a dentition of the patient can be provided for display in the UI, and/or a visualization of the first dental appliance worn over the dentition are provided for display in the UI.

In some embodiments, processing logic can provide the scan data as input to an AI model trained to provide the predicted outcome of the palatal expansion treatment plan. Processing logic can receive, as output from the AI model, the predicted outcome of the palatal expansion treatment plan.

In some embodiments, processing logic can provide the scan data as input to another AI model that is trained to provide a recommended palatal expansion treatment plan (e.g., as described with respect to FIG. 4B). The processing logic can receive, as output from the AI model, the recommended palatal expansion treatment plan, and the palatal expansion treatment plan (e.g., of block 496) can correspond to the recommended palatal expansion treatment plan.

In some embodiments, to generate the palatal expansion treatment plan, processing logic can process the 3D dental model, images, scan data, projections, etc. to obtain the target breadth of the palate, and can identify one or more intermediate breadths in the series of breadths that correspond to the progressive expansion of the palate from the initial breadth toward the target breadth. In some embodiments, the 3D visualization of the predicted outcome can include a 3D visualization of the one or more intermediate breadths. In some embodiments, the 3D visualization of the predicted outcome can include a 3D visualization of the initial breadth, and/or a 3D visualization of the target breadth.

In some embodiments, the 3D visualization of the predicted outcome is provided to a display in a user interface of the user device. Examples of a 3D visualization are described with respect to FIGS. 2, 3B. In some embodiments, the UI can include a UI element (e.g., element 260 of FIG. 2) associated with an expansion amount corresponding to the target breadth. In some embodiments, processing logic can receive, via the UI element, a modification to the expansion amount. Processing logic can generate a modified 3D visualization of the predicted outcome to correspond to the modification of the expansion amount. Processing logic can also determine a length of treatment, a number of treatment stages, etc. associated with the modified amount of expansion, and may output such information to the display. Processing logic can provide, to the user device, the modified 3D visualization (e.g., for presentation in UI portion 270 of FIG. 2).

In some embodiments, the UI can include a UI element associated an orientation of the 3D visualization. The UI element can enable a user to manipulate the orientation of the 3D visualization. For example, one or more of UI element(s) 224-240 can enable a user the orientation and/or display settings of the 3D visualization.

In some embodiments, the UI can include a first UI element associated with a measurement tool (e.g., tools 210 of FIG. 2). The first UI element can enable a user to manipulate the measurements of the 3D visualization. In some embodiments, the UI can include a second UI element associated with a measurement tool (e.g., tools 210 of FIG. 2). The second UI element can provide one or more measurements of the 3D visualization. In some embodiments, the one or more measurements can include an intercuspal jaw width (e.g., corresponding to element 216 of FIG. 2) and/or a buccal overjet measurement (e.g., corresponding to element 218 of FIG. 2).

In some embodiments, the UI can include a series of UI elements (e.g., elements 261-265 of FIG. 2). Each UI element in the series of UI elements can correspond to a palatal expansion treatment plan parameter associated with the palatal expansion treatment plan. In some embodiments, the palatal expansion treatment plan parameter corresponds to at least one of an amount of expansion, a vertical clearance measurement of an expander, a placement of an attachment on a tooth of a dentition of the patient, a first identification of at least one tooth covered by the expander, a second identification of a corresponding part of a palate of the patient covered by the expander, or a second amount of transverse force applied by the expander. Any one or more of these parameters may be adjusted via interaction with the UI. Any such adjustment may cause processing logic to update a treatment plan, including updating a final palate breadth, a treatment length, a number of treatment stages, and so on.

In some embodiments, the UI can include a UI element associated with an expansion recommendation (e.g., UI element 274 of FIG. 2) corresponding to the patient. For example, the expansion recommendation can be a part of a recommended palatal expansion treatment plan.

In some embodiments, the UI can display a first set of teeth in the 3D visualization in a first visual representation and a second set of teeth in the 3D visualization in a second visual representation. The first set of teeth can correspond to one or more teeth that are directly affected by the palatal expansion treatment plan, and the first visual representation can correspond to a first shading value. The second set of teeth can correspond to one or more teeth in indirectly affected by the palatal expansion treatment plan, and the second visual representation can correspond to a second shading value. For example, the second set of teeth can be displayed transparently, partially transparently, or opaquely.

In some embodiments, processing logic can receive (or otherwise identify) a picture of a face of the patient. In some embodiments, the picture can be received from a client device (e.g., a device of the doctor, the patient, etc.). In some embodiments, the picture can be stored in data store 108, and treatment planning system 115 can access the picture. Processing logic can generate an image of the face of the patient that represents the picture with the 3D visualization of the predicted outcome integrated into the picture. In some embodiments, processing logic can identify the inner mouth region of the picture of the patient's face, and can replace the inner mouth region with a representation of the 3D visualization of the predicted outcome. Processing logic can provide, to the user device, the image of the face of the patient.

In some embodiments, the image of the face of the patient, with the integrated 3D visualization of the predicted outcome, and the 3D visualization of the predicted outcome, can be presented in a UI side-by-side. In some embodiments, the image of the patient's face and/or the 3D visualization can correspond to a target visualization corresponding to the target breadth, an initial visualization corresponding to the initial breadth, or an intermediate visualization corresponding to a breadth between the initial breadth and the target breadth. In some embodiments, the an image or video showing the post-treatment smile of the patient is generated. In some embodiments, the post-treatment smile image is generate using a generative model, such as a generative adversarial network (GAN). In embodiments, the post-treatment smile is generated using the techniques of one or more of U.S. Pat. No. 10,980,612, issued Apr. 20, 2021, or U.S. application Ser. No. 18/525,530, filed Nov. 30, 2023, each of which is incorporated by reference herein.

In some embodiments, processing logic can provide instructions to fabricate one or more dental appliances to implement the palatal expansion treatment plan. In some embodiments, the dental appliances can be fabricated by fabrication machine(s) 170 of FIG. 1. In some embodiments, the one or more dental appliances can include a palatal expander, and/or one or more incremental palatal expanders. In some embodiments, the one or more dental appliances can be 3D printed incremental palatal expanders.

In some embodiments, processing logic can provide instructions to fabricate a series of incremental palatal expanders corresponding to the palatal expansion treatment plan. In some embodiments, the series of incremental palatal expanders can include at least one intermediate incremental palatal expander corresponding to an intermediate breadth of the palate. In some embodiments, the series of incremental palatal expanders can include a final incremental palatal expander corresponding to a final breadth of the palate. In some embodiments, one or more palatal expanders (e.g., incremental palatal expanders) are manufactured based on instructions from treatment plan.

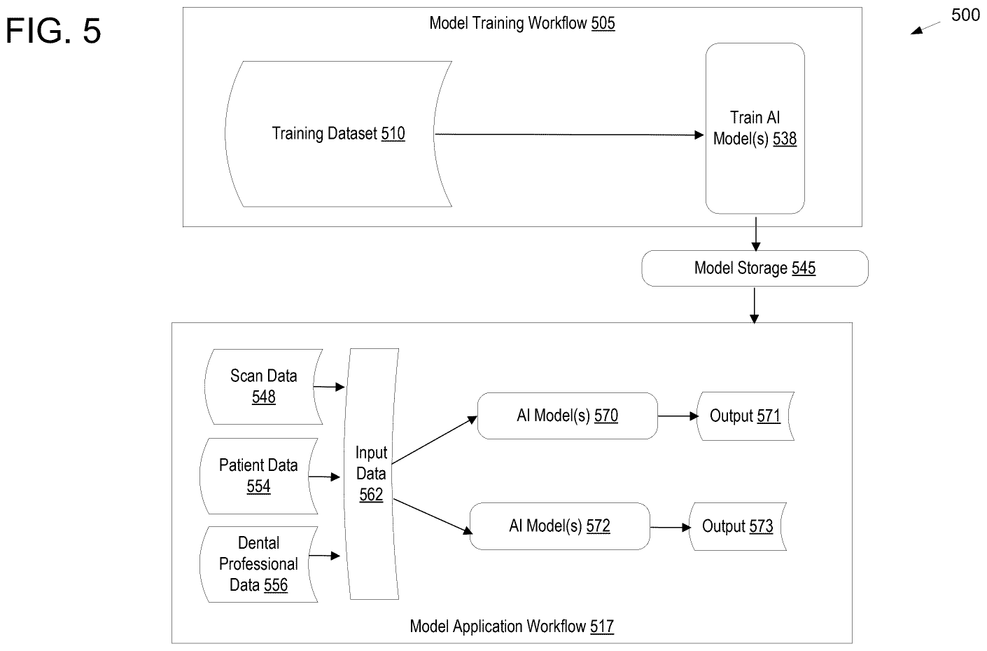

FIG. 5 illustrates workflows for training and implementing one or more AI models to provide a PE treatment plan recommendation (e.g., a recommended PE treatment plan, including, for example, he recommended palatal expansion amount), to provide indicators representing locations of references points within 3D dental models (e.g., as described with respect to transverse treatment planning component 112), and/or a PE outcome (e.g., the outcome of the particular patient's craniofacial structure following PE treatment), in accordance with some embodiments of the present disclosure. The illustrated workflows include a model training workflow 505 and a model application workflow 517. In some embodiments, the model training workflow 505 can be used to train one or more AI models (e.g., deep learning models, generative models, etc.) to provide a recommended PE treatment. The model application workflow 517 an be used to apply the one or more trained AI models to provide a recommended PE treatment (e.g., as described with respect to transverse treatment planning component 112). In some embodiments, the model training workflow 505 can be used to train one or more AI models (e.g., deep learning models, generative models, etc.) to provide indicators representing locations of reference points within 3D dental models. The model application workflow 517 an be used to apply the one or more trained AI models to provide indicators representing locations of reference points within 3D dental models (e.g., as described with respect to transverse treatment planning component 112). In some embodiments, the model workflow 505 can be used to train one or more AI models (e.g., deep learning models, generative models, etc.) to provide a predicted outcome of a PE treatment. The model application workflow 517 an be used to apply the one or more trained AI models to provide a predicted outcome of a PE treatment (e.g., as described with respect to transverse treatment outcome component 114).

One type of artificial intelligence model that may be used is an artificial neural network, such as a deep neural network. Artificial neural networks generally include a feature representation component with a classifier or regression layers that map features to a desired output space. A convolutional neural network (CNN), for example, hosts multiple layers of convolutional filters. Pooling is performed, and non-linearities may be addressed, at lower layers, on top of which a multi-layer perceptron is commonly appended, mapping top layer features extracted by the convolutional layers to decisions (e.g. classification outputs). Deep learning is a class of artificial intelligence algorithms that use a cascade of multiple layers of nonlinear processing units for feature extraction and transformation. Each successive layer uses the output from the previous layer as input. Deep neural networks may learn in a supervised (e.g., classification) and/or unsupervised (e.g., pattern analysis) manner. Deep neural networks include a hierarchy of layers, where the different layers learn different levels of representations that correspond to different levels of abstraction. In deep learning, each level learns to transform its input data into a slightly more abstract and composite representation. In an image recognition application, for example, the raw input may be a matrix of pixels; the first representational layer may abstract the pixels and encode edges; the second layer may compose and encode arrangements of edges; the third layer may encode higher level shapes (e.g., teeth, gingiva, etc.); and the fourth layer may recognize that the image contains a face or define a bounding box around teeth in the image. Notably, a deep learning process can learn which features to optimally place in which level on its own. The “deep” in “deep learning” refers to the number of layers through which the data is transformed. More precisely, deep learning systems have a substantial credit assignment path (CAP) depth. The CAP is the chain of transformations from input to output. CAPs describe potentially causal connections between input and output. For a feedforward neural network, the depth of the CAPs may be that of the network and may be the number of hidden layers plus one. For recurrent neural networks, in which a signal may propagate through a layer more than once, the CAP depth is potentially unlimited.

Training of a neural network may be achieved in a supervised learning manner, which involves feeding a training dataset consisting of labeled inputs through the network, observing its outputs, defining an error (by measuring the difference between the outputs and the label values), and using techniques such as deep gradient descent and backpropagation to tune the weights of the network across all its layers and nodes such that the error is minimized. In many applications, repeating this process across the many labeled inputs in the training dataset yields a network that can produce correct output when presented with inputs that are different than the ones present in the training dataset. In high-dimensional settings, such as large images, this generalization is achieved when a sufficiently large and diverse training dataset is made available.

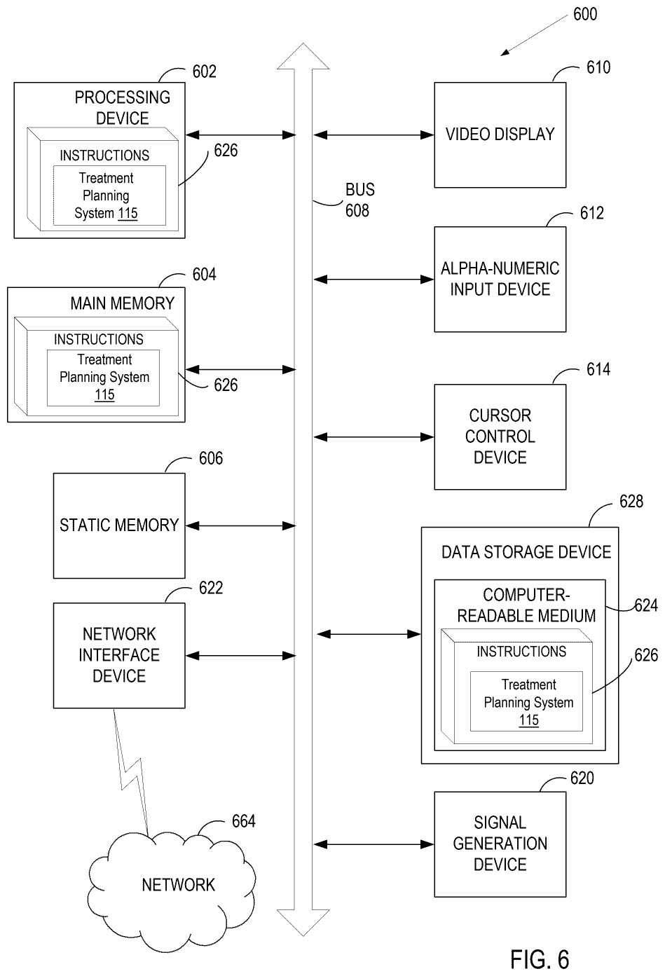

The model training workflow 505 and the model application workflow 517 may be performed by processing logic executed by a processor of a computing device (e.g., computing device 105 of FIG. 1 or a separate computing device). These workflows 505, 517 may be implemented, for example, by one or more modules executed on a processing device 602 of computing device 600 shown in FIG. 6. In some embodiments, training is performed on different devices than those that apply the trained AI models.

For the model training workflow 505, training dataset 510 containing hundreds, thousands, tens of thousands, hundreds of thousands, or more images (e.g., scan data and/or additional patient data) may be provided. Training dataset 510 can include data from a set of historic PE treatments (e.g., the set of historic PE treatments can include 2000, 3000, or more PE treatment cases). The data from the set of historic PE treatments can include scan data representing a patient's craniofacial structure before the PE treatment, and scan data representing the patient's craniofacial structure after the PE treatment. The data from the set of historic PE treatments can include the PE treatment plan for the corresponding patient, which can include the prescribed expansion amount, the actual expansion amount, the number of expanders in the treatment plan (either the number used and/or the number prescribed), the frequency of expander replacement (e.g., the amount of time the patient wore each expander), patient data (optionally with personally identifiable information removed), the placement of the attachment(s), a vertical clearance (e.g., “width” of the expander), an indication of which teeth are covered by the expander, an indication of which part of the palate is covered by the expander, an amount of transverse force applied by the expander, a treatment aggressiveness factor, the dental professional treating the patient, and/or any other applicable information. In some embodiments, the data can include indicators representing the location, in a corresponding 3D dental model, of reference point(s) (also referred to as landmarks) that are used for tooth measurements during PE treatment. In some embodiments, training dataset 510 can include multiple dataset from various view directions (e.g., corresponding to the positioning of the scanning apparatus). In some embodiments, training dataset 510 can include additional data with labels, such as mesh segment, height map (e.g., corresponding to the mesh segment), surface normal directions, occlusion data (e.g., occlusal clearance to the other jaw), color data, patient data, and/or other relevant data. In some embodiments, training dataset 510 can include labeled 3D color models, e.g., generated from intraoral scan data of the dentition of a patient and/or color 2D images. In some embodiments, the training dataset 510 can be stacked as 2D data with multiple channels. The channels can correspond to the relevant data, such as color, surface normal, occlusal distance, NIRI, etc. In some embodiments, some or all of the data may be labeled with segmentation information. The segmentation information may identify features, such as individual teeth or a region of the mouth corresponding to individual teeth.

At block 538, data from the training dataset 510 may be used to train one or more artificial intelligence models to provide a PE treatment plan, and/or to provide a PE outcome. The training dataset containing hundreds, thousands, tens of thousands, hundreds of thousands, or more data points can be used to form the training dataset 510. In embodiments, up to millions of scan data are included in a training dataset. In some embodiments, at block 538, data from training dataset 510 may be used to train one or more artificial intelligence models to generate a 3D model of the patient's dental arch(es) that represent the patient's craniofacial structure after a PE treatment.

Training may be performed by inputting one or more data points into the artificial intelligence model one at a time. The data that is input into the artificial intelligence model may include a single layer or multiple layers. In some embodiments, a recurrent neural network (RNN) is used. In such an embodiment, a second layer may include a previous output of the artificial intelligence model (which resulted from processing a previous input).

The artificial intelligence model processes the input to generate an output. An artificial neural network includes an input layer that consists of values in a data point. The next layer is called a hidden layer, and nodes at the hidden layer each receive one or more of the input values. Each node contains parameters (e.g., weights) to apply to the input values. Each node therefore essentially inputs the input values into a multivariate function (e.g., a non-linear mathematical transformation) to produce an output value. A next layer may be another hidden layer or an output layer. In either case, the nodes at the next layer receive the output values from the nodes at the previous layer, and each node applies weights to those values and then generates its own output value. This may be performed at each layer. A final layer is the output layer, where there is one node for each class, prediction and/or output that the artificial intelligence model can produce. For example, for an artificial neural network being trained to output a PE treatment plan, and/or to output an outcome of a predicted PE treatment.

Processing logic may then compare the generated outputs to the known condition and/or label that was included in the training data item. Processing logic determines an error based on the differences between the output probability map and/or label(s) and the provided probability map and/or label(s). Processing logic adjusts weights of one or more nodes in the artificial intelligence model based on the error. An error term or delta may be determined for each node in the artificial neural network. Based on this error, the artificial neural network adjusts one or more of its parameters for one or more of its nodes (the weights for one or more inputs of a node). Parameters may be updated in a back propagation manner, such that nodes at a highest layer are updated first, followed by nodes at a next layer, and so on. An artificial neural network contains multiple layers of “neurons,” where each layer receives input values from neurons at a previous layer. The parameters for each neuron include weights associated with the values that are received from each of the neurons at a previous layer. Accordingly, adjusting the parameters may include adjusting the weights assigned to each of the inputs for one or more neurons at one or more layers in the artificial neural network.

Once the model parameters have been optimized, model validation may be performed to determine whether the model has improved and to determine a current accuracy of the model. After one or more rounds of training, processing logic may determine whether a stopping criterion has been met. A stopping criterion may be a target level of accuracy, a target number of processed data items from the training dataset, a target amount of change to parameters over one or more previous data points, a combination thereof and/or other criteria. In one embodiment, the stopping criteria is met when at least a minimum number of data points have been processed and at least a threshold accuracy is achieved. The threshold accuracy may be, for example, 70%, 80% or 90% accuracy. In one embodiment, the stopping criteria is met if accuracy of the artificial intelligence model has stopped improving. If the stopping criterion has not been met, further training is performed. If the stopping criterion has been met, training may be complete. Once the artificial intelligence model is trained, a reserved portion of the training dataset 510 may be used to test the model. Testing the model can include performing unit tests, regression tests, and/or integration tests.

Once one or more trained AI models are generated, they may be stored in model storage 545. Multiple AI models can be trained and used in combination. For example, model training workflow 505 can train an AI model to provide one or more PE treatment plans, an AI model to provide a predicted outcome of a PE treatment plan, and/or a generative AI model to generate a 3D model of the patient's craniofacial structure corresponding to the predicted outcome of the PE treatment plan. In some embodiments, a first AI model can output value(s) indicating parameter values corresponding to a recommended PE treatment plan. The value(s) can indicate, for example, the recommended expansion amount, the number of expanders in the treatment plan, etc. In some embodiments, a second AI model can output value(s) indicating a predicted outcome of a particular PE treatment plan. The value(s) can indicate, for example, the expansion amount, the placement of one or more teeth in the upper jaw, the shape of the palate, etc. In some embodiments, the value(s) can be used to generate a 3D model and/or 2D cross-section view of the patient's craniofacial structure following the PE treatment. In some embodiments, the generative AI model can output a 3D model of the patient's craniofacial structure and/or dental arch that illustrates the predicted outcome of the PE treatment. In some embodiments, a third AI model can output value(s) indicating location(s) of reference point(s) within a 3D dental model. The value(s) can represent coordinates corresponding to the 3D dental model, indicating the location of reference points that can be used in determining tooth measurements (which can in turn be used to determine a recommended palatal expansion amount and/or a recommended palatal expansion treatment plan). The reference points can be referred to as landmarks herein.

In some embodiments, model application workflow 517 includes one or more trained AI model(s) 570. These logics may be implemented as separate artificial intelligence models or as a single combined artificial intelligence model, in embodiments. However, each of these logics may include distinct higher level layers of the deep neural network that are trained to generate different types of outputs.

In some embodiments, a patient, a dental professional (e.g., a doctor, dentist, hygienist, or technician), and/or another individual may perform a scan of a patient's dental arch(es) at a single point in time (e.g., during a visit to the dentist). The scan can be, for example, an intraoral scan, e.g., as described with respect to FIG. 1. The data generated by the scan can be preprocessed to segment the scan data. The data generated by the scan, optionally preprocessed, can correspond to scan data 548, and/or scan data of scan data store 144 of FIG. 1.

In some embodiments, the dental professional may have previously captured a scan (e.g., intraoral scan, CBCT scan, etc.), and/or may have other patient data, such as the patient's chart, the patient's previous diagnoses, the patient's previous treatments, the patient's answers to a questionnaire (optionally including a history of patient's answers), 2D image data of the patient's smile showing their teeth (e.g., as captured by a mobile computing device of the patient), and/or the patient's occlusion data, which may correspond to patient data 554. In some embodiments, the dental professional may provide dental professional data 556 that can include, for example, historical PE treatment case data of that dental professional. In some embodiments, dental professional data 556 can include data input via the UI (e.g., UI 200 of FIG. 2). Scan data 548, patient data 554, and/or dental professional data 556 may be combined to form input data 562. In some embodiments, some or all of input data 562 may be processed by a segmenter, which may produce segmentation information, e.g., identifying individual teeth or regions in the dental arch corresponding to individual teeth. The segmentation information can be included in input data 562.

In some embodiments, the treatment planning system 115 can provide an updated treatment plan recommendation and/or a predicted outcome during the execution of a PE treatment plan. In such instances, the input data 562 can include current scan data of the patient, the original 3D model and/or scan data of the patient (e.g., representing the patient's craniofacial structure at the start of, or before, the PE treatment began), and/or a predicted 3D model (or predicted outcome value(s)) for the current stage of the PE treatment. As an illustrative example, the treatment planning system 115 can provide an updated treatment plan recommendation and/or predicted outcome for a patient partway through the PE treatment plan (e.g., at 3 mm of expansion during a 6 mm expansion treatment plan, at expander 15 of a 30-expander treatment plan, or at day 15 of a 30-day treatment plan, etc.). In such cases, the input data 562 can include data of the patient's current craniofacial structure (e.g., a photo received from the patient's phone), the predicted outcome at this stage of the treatment, and/or the original scan data of the patient. For example, the current data of the patient's craniofacial structure can include the amount of expansion thus far during the treatment. The AI model(s) 570, 572 can provide, as output, an updated treatment plan recommendation and/or an updated predicted outcome of the patient's craniofacial structure, e.g., based on the current state of the patient's craniofacial structure thus far during the treatment.

Input data 562 can be provided as input to AI model(s) 570 and/or AI model(s) 572. AI model(s) 570 may produce output 571, which may include value(s) indicating a recommended PE treatment. In some embodiments, AI model(s) 570 can include two (or more) AI models, and the output of each AI model may be aggregated to provide a single output. AI model(s) 572 can produce output 573, which may include value(s) indicating a predicted outcome of a PE treatment plan. In some embodiments, the value(s) can include the arch width, the occlusal contacts, tooth tipping/angle, and/or additional values indicating the predicted future state of the teeth after the PE treatment plan. In some embodiments, AI model(s) 572 can include two (or more) AI models, the output of each AI model may be aggregated to provide a single output. In some embodiments, AI model(s) 572 can include a generative AI model that outputs a 3D model of a predicted outcome of the patient's craniofacial structure following a PE treatment plan.

In some embodiments, AI model(s) 572 can include a generative model that is trained to generate a predicted 3D model of the future condition of the patient's craniofacial structure (including, e.g., the upper arch, and optionally the lower arch) after having undergone a PE treatment. For example, AI model(s) 572 can include a generative adversarial network (GAN). A GAN is a class of artificial intelligence system that uses two artificial neural networks contesting with each other in a zero-sum game framework. The GAN includes a first artificial neural network that generates candidates (e.g., for post-treatment faces of patients) and a second artificial neural network that evaluates the generated candidates. The GAN learns to map from a latent space to a particular data distribution of interest (a data distribution of changes to input images that are indistinguishable from photographs to the human eye), while the discriminative network discriminates between instances from a training dataset and candidates produced by the generator. The generative network's training objective is to increase the error rate of the discriminative network (e.g., to fool the discriminator network by producing novel synthesized instances that appear to have come from the training dataset). The generative network and the discriminator network are co-trained, and the generative network learns to generate images that are increasingly more difficult for the discriminative network to distinguish from real images (from the training dataset) while the discriminative network at the same time learns to be better able to distinguish between synthesized images and images from the training dataset. The two networks of the GAN are trained once they reach equilibrium. The GAN may include a generator network that generates a 3D model of the predicted outcome of the patient's craniofacial structure after the PE treatment. In some embodiments, the generative AI model can receive, as input, scan data 562 (e.g., including a 3D model of the patient), and can generate a 3D model of the predicted outcome of the patient's craniofacial structure.

In some embodiments, AI model(s) 572 can include an AI model that receives, as input, the scan data of the patient and the output of another AI model(s) 572 (e.g., output 573), which may include value(s) indicating a predicted outcome of a PE treatment plan, and/or generates a 3D model of the predicted outcome of the patient's craniofacial structure post-PE treatment. In some embodiments, the output 573 can include data that can be used by transverse treatment visualization component 116 to generate a 3D model of the predicted outcome of the patient's craniofacial structure post-PE treatment (e.g., spatial data, mesh data, color data, and so on).

FIG. 6 illustrates a diagrammatic representation of a machine in the example form of a computing device 600 within which a set of instructions, for causing the machine to perform any one or more of the methodologies discussed herein, may be executed. In alternative embodiments, the machine may be connected (e.g., networked) to other machines in a Local Area Network (LAN), an intranet, an extranet, or the Internet. The machine may operate in the capacity of a server or a client machine in a client-server network environment, or as a peer machine in a peer-to-peer (or distributed) network environment. The machine may be a personal computer (PC), a tablet computer, a set-top box (STB), a Personal Digital Assistant (PDA), a cellular telephone, a web appliance, a server, a network router, switch or bridge, or any machine capable of executing a set of instructions (sequential or otherwise) that specify actions to be taken by that machine. Further, while only a single machine is illustrated, the term “machine” shall also be taken to include any collection of machines (e.g., computers) that individually or jointly execute a set (or multiple sets) of instructions to perform any one or more of the methodologies discussed herein. In one embodiment, the computing device 600 corresponds to any computing device of FIG. 1.

The example computing device 600 includes a processing device 602 (e.g., a CPU), a main memory 604 (e.g., read-only memory (ROM), flash memory, dynamic random access memory (DRAM) such as synchronous DRAM (SDRAM), etc.), a static memory 606 (e.g., flash memory, static random access memory (SRAM), etc.), and a secondary memory (e.g., a data storage device 628), which communicate with each other via a bus 608.

Processing device 602 represents one or more general-purpose processors such as a microprocessor, central processing unit, or the like. More particularly, the processing device 602 may be a complex instruction set computing (CISC) microprocessor, reduced instruction set computing (RISC) microprocessor, very long instruction word (VLIW) microprocessor, processor implementing other instruction sets, or processors implementing a combination of instruction sets. Processing device 602 may also be one or more special-purpose processing devices such as an application specific integrated circuit (ASIC), a field programmable gate array (FPGA), a digital signal processor (DSP), network processor, or the like. In accordance with one or more aspects of the present disclosure, processing device 602 is configured to execute the processing logic (instructions 626, which may implement the treatment planning system 115 of FIG. 1) for performing operations and steps discussed herein. While only a single example processing device is illustrated, the term “processing device” shall also be taken to include any collection of processing devices (e.g., computers) that individually or jointly execute a set (or multiple sets) of instructions to perform any one or more of the methods discussed herein.

The computing device 600 may further include a network interface device 622 for communicating with a network 664. The computing device 600 also may include a video display unit 610 (e.g., a liquid crystal display (LCD) or a cathode ray tube (CRT)), an alphanumeric input device 612 (e.g., a keyboard), a cursor control device 614 (e.g., a mouse, a touch-screen control device), and a signal generation device 620 (e.g., a speaker).

The data storage device 628 may include a machine-readable storage medium (or more specifically a non-transitory computer-readable storage medium) 624 on which is stored one or more sets of instructions 626 embodying any one or more of the methodologies or functions described herein. A non-transitory storage medium refers to a storage medium other than a carrier wave. The instructions 626 may also reside, completely or at least partially, within the main memory 604 and/or within the processing device 602 during execution thereof by the computer device 600, the main memory 604 and the processing device 602 also constituting computer-readable storage media.

The computer-readable storage medium 624 may also be used to store a treatment planning system 115, which may correspond to the similarly named component of FIG. 1. The computer readable storage medium 624 may also store a software library containing methods for a treatment planning system 115. While the computer-readable storage medium 624 is shown in an example embodiment to be a single medium, the term “computer-readable storage medium” should be taken to include a single medium or multiple media (e.g., a centralized or distributed database, and/or associated caches and servers) that store the one or more sets of instructions. The term “computer-readable storage medium” shall also be taken to include any non-transitory medium (e.g., a medium other than a carrier wave) that is capable of storing or encoding a set of instructions for execution by the machine and that cause the machine to perform any one or more of the methodologies of the present disclosure. The term “computer-readable storage medium” shall accordingly be taken to include, but not be limited to, solid-state memories, and optical and magnetic media.

Any of the methods (including user interfaces) described herein may be implemented as software, hardware or firmware, and may be described as a non-transitory machine-readable storage medium storing a set of instructions capable of being executed by a processor (e.g., computer, tablet, smartphone, etc.), that when executed by the processor causes the processor to control perform any of the steps, including but not limited to: displaying, communicating with the user, analyzing, modifying parameters (including timing, frequency, intensity, etc.), determining, alerting, or the like. For example, computer models (e.g., for additive manufacturing) and instructions related to forming a dental device may be stored on a non-transitory machine-readable storage medium.

It should be understood that the above description is intended to be illustrative, and not restrictive. Many other embodiment examples will be apparent to those of skill in the art upon reading and understanding the above description. Although the present disclosure describes specific examples, it will be recognized that the systems and methods of the present disclosure are not limited to the examples described herein, but may be practiced with modifications within the scope of the appended claims. Accordingly, the specification and drawings are to be regarded in an illustrative sense rather than a restrictive sense. The scope of the present disclosure should, therefore, be determined with reference to the appended claims, along with the full scope of equivalents to which such claims are entitled.

The embodiments of methods, hardware, software, firmware, or code set forth above may be implemented via instructions or code stored on a machine-accessible, machine readable, computer accessible, or computer readable medium which are executable by a processing element. “Memory” includes any mechanism that provides (i.e., stores and/or transmits) information in a form readable by a machine, such as a computer or electronic system. For example, “memory” includes random-access memory (RAM), such as static RAM (SRAM) or dynamic RAM (DRAM); ROM; magnetic or optical storage medium; flash memory devices; electrical storage devices; optical storage devices; acoustical storage devices, and any type of tangible machine-readable medium suitable for storing or transmitting electronic instructions or information in a form readable by a machine (e.g., a computer).

Reference throughout this specification to “one embodiment” or “an embodiment” means that a particular feature, structure, or characteristic described in connection with the embodiment is included in at least one embodiment of the disclosure. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

In the foregoing specification, a detailed description has been given with reference to specific exemplary embodiments. It will, however, be evident that various modifications and changes may be made thereto without departing from the broader spirit and scope of the disclosure as set forth in the appended claims. The specification and drawings are, accordingly, to be regarded in an illustrative sense rather than a restrictive sense. Furthermore, the foregoing use of embodiment, embodiment, and/or other exemplarily language does not necessarily refer to the same embodiment or the same example, but may refer to different and distinct embodiments, as well as potentially the same embodiment.

The words “example” or “exemplary” are used herein to mean serving as an example, instance, or illustration. Any aspect or design described herein as “example’ or “exemplary” is not necessarily to be construed as preferred or advantageous over other aspects or designs. Rather, use of the words “example” or “exemplary” is intended to present concepts in a concrete fashion. As used in this application, the term “or” is intended to mean an inclusive “or” rather than an exclusive “or.” That is, unless specified otherwise, or clear from context, “X includes A or B” is intended to mean any of the natural inclusive permutations. That is, if X includes A; X includes B; or X includes both A and B, then “X includes A or B” is satisfied under any of the foregoing instances. In addition, the articles “a” and “an” as used in this application and the appended claims should generally be construed to mean “one or more” unless specified otherwise or clear from context to be directed to a singular form. Moreover, use of the term “an embodiment” or “one embodiment” or “an embodiment” or “one embodiment” throughout is not intended to mean the same embodiment or embodiment unless described as such. Also, the terms “first,” “second,” “third,” “fourth,” etc. as used herein are meant as labels to distinguish among different elements and may not necessarily have an ordinal meaning according to their numerical designation.

A digital computer program, which may also be referred to or described as a program, software, a software application, a module, a software module, a script, or code, can be written in any form of programming language, including compiled or interpreted languages, or declarative or procedural languages, and it can be deployed in any form, including as a stand-alone program or as a module, component, subroutine, or other unit suitable for use in a digital computing environment. The essential elements of a digital computer a central processing unit for performing or executing instructions and one or more memory devices for storing instructions and digital data. The central processing unit and the memory can be supplemented by, or incorporated in, special purpose logic circuitry or quantum simulators. Generally, a digital computer will also include, or be operatively coupled to receive digital data from or transfer digital data to, or both, one or more mass storage devices for storing digital data, e.g., magnetic, magneto-optical disks, optical disks, or systems suitable for storing information. However, a digital computer need not have such devices.

Digital computer-readable media suitable for storing digital computer program instructions and digital data include all forms of non-volatile digital memory, media, and memory devices, including by way of example semiconductor memory devices, e.g., EPROM, EEPROM, and flash memory devices; magnetic disks, e.g., internal hard disks or removable disks; magneto-optical disks; CD-ROM and DVD-ROM disks.

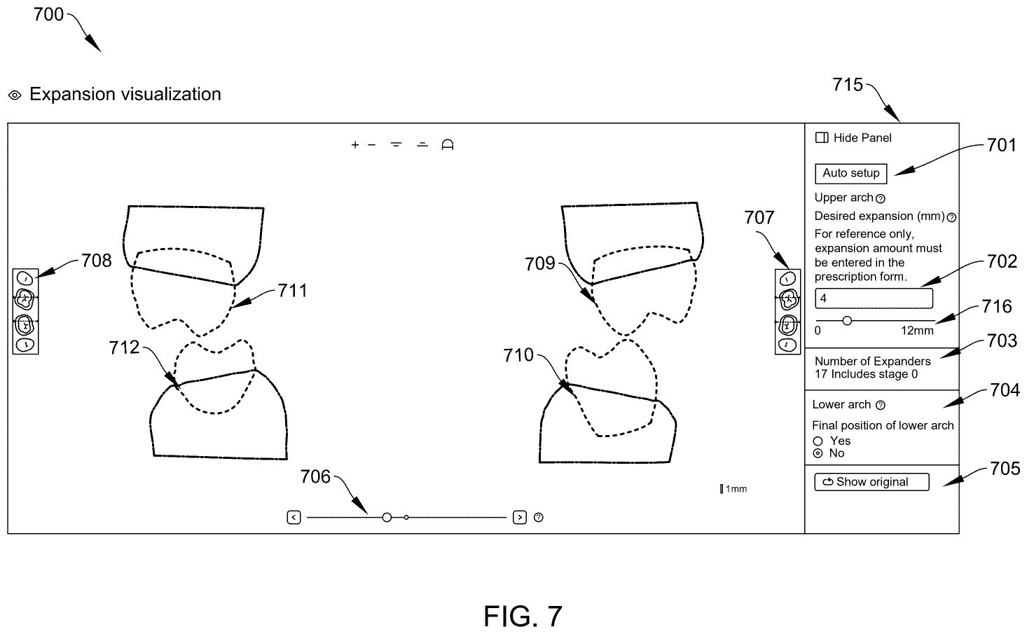

FIG. 7 illustrates a portion 700 of an example user interface of a palatal expansion previewer and treatment management tool displaying an “auto setup” feature, in accordance with some embodiments of the present disclosure. The “auto setup” feature automatically calculates the amount of upper arch expansion needed, e.g., based on a final position of the lower jaw. In some embodiments, the “auto setup” feature enables dental professionals to automatically apply the recommended expansion amount, e.g., based on the algorithms' analysis of anatomical landmarks, such as the positions of upper and lower molar reference points. The “auto setup” feature is illustrated as button 701 in UI 700. In some embodiments, when a user (e.g., a dental professional) clicks on the “auto setup” button 701, the transverse treatment planning component 112 of FIG. 1 can determine the recommended expansion amount and recommended treatment plan based on the position of the lower jaw. The transverse treatment visualization component 116 of FIG. 1 can update the UI 700 to reflect the recommended expansion amount and treatment plan, including updating the amount of expansion and the number of expanders 703. In some embodiments, the amount of expansion can be modified by a user (e.g., a dental practitioner) using the desired expansion textbox 702 and/or the desired expansion slider tool 716. When a user modifies the desired expansion, the transverse treatment visualization component 116 can automatically update UI 700 to reflect the corresponding number of expanders 703, and/or the positions of the teeth.

In some embodiments, UI 700 (or a portion of UI 700) can be displayed on a computing device of a patient and/or a doctor (e.g., corresponding to computing device 160 of FIG. 1), and/or on a scanning device (e.g., of oral state capture system(s) 110 of FIG. 1). In some embodiments, the UI can correspond to UI 200 of FIG. 2, and the portion 700 can correspond to portion 270 of FIG. 2. In some embodiments, the UI can correspond to UI portion 370 of FIGS. 3A, 3B.

In some embodiments, UI 700 can include a panel 715 that includes the auto setup button 701, the amount of expansion 702, the number of expanders 703, as well as a “show original” button 705 and a lower arch final position indicator 704. In some embodiments, a user (e.g., a dental practitioner) can use lower arch final position indicator 704 to indicate to the system whether the final position of the lower arch is expected to change, e.g., due to an ongoing or planned orthodontic treatment plan. In some embodiments, the “show original” button 705 can enable a user to revert the 2D cross-sections displayed in UI portion 700 to the original version, e.g., before the dental professional made modifications to the treatment plane (e.g., before the dental professional modified the desired expansion amount). In some embodiments, UI 700 can also include side panels 707 and 708, as well as a sliding bar 706. Side panels 707 and 708 can display representations and/or images of the teeth that include dotted lines depicting where the cross-section intersects the teeth to generate the 2D cross-sectional view displayed in UI 700. For example, side panel 707 displays a representation of the upper right second bicuspid (or second premolar), the upper right first molar, the lower right first molar, and the lower right second bicuspid (or second premolar), and includes dotted lines across the upper right first molar and the lower right first molar. The dotted lines represent where the cross-section intersects the upper right first molar and the lower right first molar to generate the cross-sectional view of the upper right first molar 709 and the lower right first molar 710. Similarly, side panel 708 displays a representation of the upper left second bicuspid (or second premolar), the upper left first molar, the lower left first molar, and the lower left second bicuspid (or second premolar), and includes dotted lines across the upper left first molar and the lower left first molar. The dotted lines represent where the cross-section intersects the upper left first molar and the lower left first molar to generate the cross-sectional view of the upper left first molar 711 and the lower left first molar 712. The sliding bar 706 can enable a user (e.g., a dental professional) to change the 2D cross-section position along the teeth. For example, by sliding the handle along sliding bar 706 to the left, the dotted lines (represented in panels 707 and 708) intersecting the first molars can move in one direction (e.g., toward the back of the mouth), and by sliding the handle along the sliding bar 706 to the right, the dotted lines (represented in panels 707 and 708) intersecting the first molars can move in another direction (e.g., toward the front of the mouth).

FIG. 8 illustrates a portion 800 of an example user interface of a palatal expansion previewer and treatment management tool displaying reference points, in accordance with some embodiments of the present disclosure. In some embodiments, the reference points can correspond to anatomical landmarks used in the upper arch expansion determination (e.g., using the “auto setup” feature of FIG. 7). As illustrated in FIG. 8, the portion 800 of the UI can include a 3D view and a 2D cross-sectional view of the patient's teeth. In embodiments, the 3D view and 2D cross-sectional view may be presented in different regions of the UI (e.g., in different regions of a display on a client device). In some embodiments, the reference points can be displayed in different colors to enable the user to correlate a reference point displayed in the 2D cross-sectional view and corresponding reference point displayed in the 3D view. For example, reference points 801 and 802 can both be displayed in to blue to denote that they are the same reference point illustrated in the 3D view (for reference point 801) and 2D view (for reference point 802).

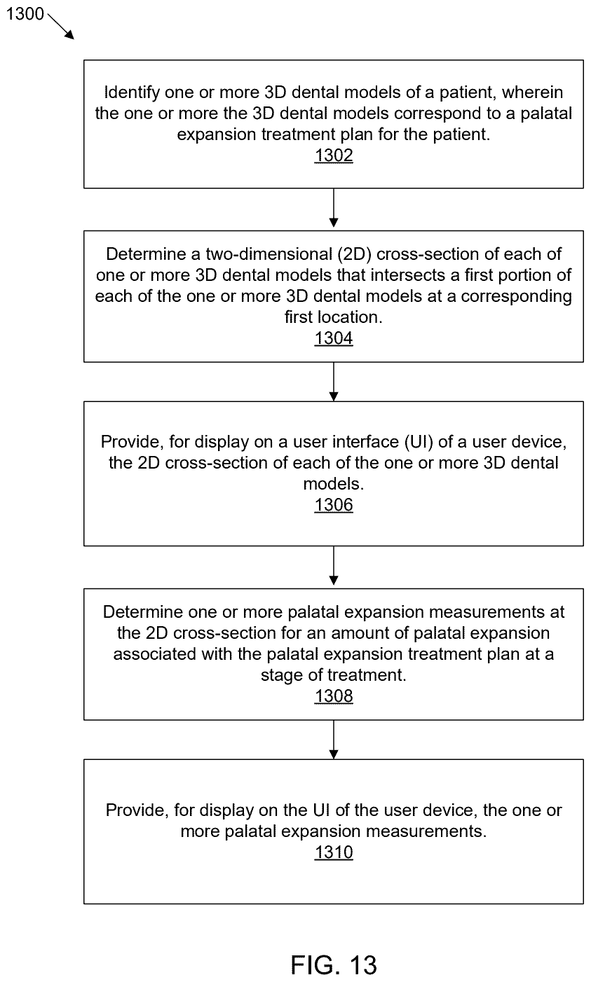

In some embodiments, the reference points (e.g., reference points 801-802) can be predetermined (e.g., by transverse treatment planning component 112) as being relevant to PE treatment planning. Additionally or alternatively, the reference points (e.g., reference points 801-802) can be selected by a user (e.g., a dental professional). For example, a user can click on a point on a tooth displayed in the UI (e.g., UI portion 800), and identify that point as a reference point. In some embodiments, the user can select the reference point(s) from a dropdown menu. The reference points illustrated in FIG. 8 correspond to the molar cusps. Other relevant reference points include, for example, groove center(s), closest cementoenamel junction (CEJ) point(s), closest jaw bone point(s), and so on. For example, the reference points can correspond to the transpalatal width of the maxillary arch, which can be measured between the closest point of the upper first molars (CEJ points). The closest CEJ points of the upper first molars can refer to the shortest linear distance between the CEJ point of the left upper first molar and the CEJ point of the right upper first molar. As another example, the reference points can correspond to a coronal projection of a CBCT image measuring the distance between the closest jaw bone points. The closest jaw bone points can refer to the shortest distance between the jaw bone cortices across the arch (e.g., across the upper arch). In some embodiments, UI 800 illustrates a final stage of an expansion treatment plan where the upper lingual molar cusps 804 barely touch the lower buccal molar cusps 805 when the jaw is closed. An example method to determine the final stage of a treatment plan and the corresponding recommended expansion amount is described with respect to FIG. 13.

In some embodiments, UI 800 (or a portion of UI 800) can be displayed on a computing device of a patient and/or a doctor (e.g., corresponding to computing device 160 of FIG. 1), and/or on a scanning device (e.g., of oral state capture system(s) 110 of FIG. 1). In some embodiments, operations are performed on a server computing device (e.g., which may execute in a cloud computing environment), and may be sent to a client computing device or a patient and/or doctor for review and/or manipulation. In some embodiments, the UI can correspond to UI 200 of FIG. 2, and the portion 800 can correspond to portion 270 of FIG. 2.

In some embodiments, UI 800 can include a side panel 808 that includes a representation and/or an image of the patient's teeth, with a dotted line representing where the cross-section intersects the teeth to generate the 2D cross-sectional view displayed in UI 800. As an illustrative example, side panel 808 displays a representation of the upper left second bicuspid (or second premolar), the upper left first molar, the lower left first molar, and the lower left second bicuspid (or second premolar), and includes dotted lines across the upper left first molar and the lower left first molar. In some embodiments, UI 800 can include another side panel (not illustrated) displaying a representation of the upper right second bicuspid (or second premolar), the upper right first molar, the lower right first molar, and the lower right second bicuspid (or second premolar), and includes dotted lines across the upper right first molar and the lower right first molar (e.g., similar to side panel 707 of FIG. 7). In some embodiments, the dotted lines displayed in the side panel(s) (e.g., side panel 808) can correspond to the dotted line 809 in the 3D portion of UI 800, displaying where the cross-section intersects the 3D dental model to generate the 2D cross-sectional view of the teeth displayed in UI 800.

FIG. 9 illustrates a portion 900 of an example user interface of a palatal expansion previewer and treatment management tool displaying occlusal view images of a patient's teeth along with two-dimensional cross-sectional visualizations of teeth, in accordance with some embodiments of the present disclosure.

As illustrated in FIG. 9, the UI portion 900 includes real images 901-904 of the patient's teeth, captured in an occlusal (top-down) view. As an illustrative example, image 901 shows the patient's upper right first molar, upper right second bicuspid (second premolar), upper right first bicuspid (first premolar); image 902 shows the patient's lower right first molar, lower right second bicuspid (second premolar), lower right first bicuspid (first premolar); image 903 shows the patient's the upper left first molar, upper left second bicuspid (second premolar), upper left first bicuspid (first premolar); and image 904 shows the patient's lower left first molar, lower left second bicuspid (second premolar), lower left first bicuspid (first premolar). UI portion 900 displays 2D cross-sectional views of the patient's first molars, e.g., upper right first molar 907, lower right first molar 910, upper left first molar 911, and lower left first molar 912. Also included in each image 901-904 is a line depicting the 2D cross-sectional plane used to generate the 2D view of the teeth. For example, dotted line 906 depicts where the plane intersects the first molar depicted in image 901 to generate the 2D cross-sectional view 907 of the patient's upper right first molar. Displaying real images 901-904 of the patient's teeth provides a more accurate and patient-specific reference for the cross-sectional plane's position.

In some embodiments, UI 900 can include a customizable table 908 that lists palatal expansion arch width measurements. Table 908 can include upper and lower arch initial measurements, desired/target measurements, and the difference between the two measurements. The measurements can include, for example, the average posterior crown center measurement, the first permanent molar distolingual cusp measurement, the first permanent molar mesiolingual cusp measurement, and/or the first permanent molar lingual CEJ measurement. For example, the average posterior crown center measurement can reflect the distance between the average position of the posterior teeth crown centers on one side (e.g., the right side) and the average position of the posterior teeth crown centers on the opposite side (e.g., the left side); the first permanent molar distolingual cusp measurement can reflect the distance between the first permanent molar distolingual cusp on one side (e.g., the right side) and the first permanent molar distolingual cusp on the other side (e.g., the left side); and so on. The initial measurement can correspond to the patient's state prior to palatal expansion treatment, and the desired measurement can represent the final treatment stage of the recommended palatal expansion treatment plan. For example, as illustrated in FIG. 9, the expansion amount of the recommended treatment plan can be 5.5 millimeters, and the recommended treatment plan can include 23 expanders.

In some embodiments, UI 900 can include a sliding bar 910 that can enable a user to change the cross-sectional plane positions (e.g., represented by dotted line 906). In some embodiments, a user can move the position of the cross-sectional plane by clicking on a tooth portion within the images 901-904, and/or by dragging the dotted line 906 representing the location of the cross-section plane to a different location. The 2D cross-sectional view 907 can be automatically updated to reflect the updated cross-section location.

In some embodiments, UI 900 (or a portion of UI 900) can be displayed on a computing device of a patient and/or a doctor (e.g., corresponding to computing device 160 of FIG. 1), and/or on a scanning device (e.g., of oral state capture system(s) 110 of FIG. 1). In some embodiments, the UI can correspond to UI 200 of FIG. 2, and the UI portion 900 can correspond to portion 270 of FIG. 2. Also included in UI portion 900 is a side panel 915, that includes the same or similar features as side panel 715 of FIG. 7.

FIG. 10 illustrates a portion 1000 of an example user interface of a palatal expansion previewer and treatment management tool displaying the visualization of a cross-sectional plane with optional indicators of reference points, in accordance with some embodiments of the present disclosure. In some embodiments, UI portion 1000 can have the same or similar features as UI 700 of FIG. 7, and/or UI 900 of FIG. 9. For example, UI 1000 can include a side panel 1015 that includes the same or similar features as side panel 715 of FIG. 7 and/or side panel 915 of FIG. 9, a sliding bar similar to sliding bar 706 of FIG. 7 and/or sliding bar 910 of FIG. 9, side panels similar to side panels 901, 902, 903, 904 of FIG. 9, and so on.

In some embodiments, UI 1000 can include (in addition to or instead of table 908 of FIG. 9) a customizable table 1002 that lists various reference points (e.g., landmarks) and reference point measurements. For example, table 1002 can list the average crown center for both the upper and lower arches at an initial stage (e.g., before expansion treatment) and a desired stage (e.g., a final or target stage, as illustrated in FIG. 10, corresponding to 5 millimeters of expansion (1021) and 21 expanders (1022)). Table 1002 can also list additional landmarks, such as lingual distal landmark of the first molar cusp, lingual mesial landmark of the first molar cusp, and/or the CEJ landmark. The measurements in table 1002 can reflect the distance between the landmarks on either side. For example, the measurement for average crown center can reflect the distance between the average position of the crown centers on one side (e.g., the right side) and the average position of the crown centers on the opposite side (e.g., the left side); the first molar lingual distal cusp measurement can reflect the distance between the first molar lingual distal cusp on one side (e.g., the right side) and the first molar lingual distal cusp on the other side (e.g., the left side); and so on. Sub-table 1010 displays a customization option for table 1002. A user can customize table 1002 by adding or removing landmarks, showing (or not showing) the additional landmarks on the lower arch, and/or showing (or not showing) the landmark visualizations. Other customizations can be envisioned for table 1002 and UI 1000.

In some embodiments, the reference point(s) can be visualized on the cross-sectional image (e.g., cross-sectional image 1005) and/or on the occlusal view teeth images (e.g., occlusal view image 1006). As an illustrative example, the lingual mesial landmark of the first molar cusp can be depicted on the cross-sectional image 1005 and on the occlusal view teeth image 1006 using an upward facing purple arrow. Thus, in some embodiments, two orthogonal projections of the reference points can be visible at the same time, making their position in 3D space clear to the user.

In some embodiments, a user can select which landmark(s) and/or reference point(s) to visualize in UI 1000, and/or which landmark(s) or reference point(s) to use in the determination of the palatal expansion treatment plan, using UI 1000. For example, a user can select a landmark or reference point to visualize and/or use by clicking on the relevant point on a tooth, and/or by selecting the reference point and/or landmark from a dropdown menu (not pictured).

In some embodiments, UI 1000 (or a portion of UI 1000) can be displayed on a computing device of a patient and/or a doctor (e.g., corresponding to computing device 160 of FIG. 1), and/or on a scanning device (e.g., of oral state capture system(s) 110 of FIG. 1). In some embodiments, the UI can correspond to UI 200 of FIG. 2, and the UI portion 1000 can correspond to portion 270 of FIG. 2.

FIG. 11 illustrates a portion 1100 of an example user interface of a palatal expansion previewer and treatment management tool that includes a ruler tool 1101, in accordance with some embodiments of the present disclosure. In some embodiments, the ruler tool 1101 can correspond to ruler 214 of FIG. 2. In some embodiments, UI 1100 displays the distance between reference points (e.g., 1102). For example, the distance between upper-right tooth number 6 (UR6) and upper left tooth number 6 (UL6) is 42.9 millimeters, and the distance between upper right tooth number 5 (UR5) and upper left tooth number 5 (UL5) is 40.7 millimeters. In some embodiments, UR6 can represent the midpoint between lingual cusps upper right number 6, and UR6 can represent the midpoint between the buccal cusps of the lower right too number 6. Similarly, in some embodiments, UR5 can represent the midpoint between lingual cusps upper right number 5, and UR5 can represent the midpoint between the buccal cusps of the lower right too number 5. The ruler tool 1101 and displayed measurements of UI 1100 can make it easier for a user to distinguish between reference point types. In some embodiments, UI 1100 can include reference point indicators (e.g., 1103). The reference point indicators can show surface and/or internal points.

In some embodiments, UI 1100 (or a portion of UI 1100) can be displayed on a computing device of a patient and/or a doctor (e.g., corresponding to computing device 160 of FIG. 1), and/or on a scanning device (e.g., of oral state capture system(s) 110 of FIG. 1). In some embodiments, the UI can correspond to UI 200 of FIG. 2, and the UI portion 1100 can correspond to portion 270 of FIG. 2.

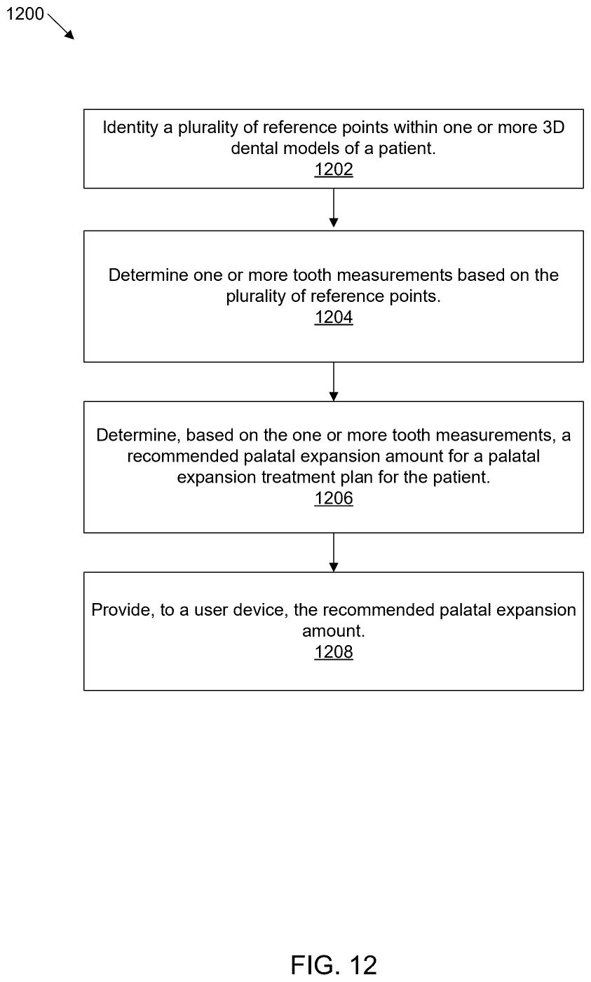

FIG. 12 illustrates a flow diagram of an example method 1200 for determining an upper arch expansion amount based on a lower arch final position, in accordance with some embodiments of the present disclosure. Method 1200 may be performed by a processing device that may include hardware, software, or a combination of both. The processing device may include one or more central processing units (CPUs), graphics processing units (GPUs), field-programmable gate arrays (FPGAs), application-specific integrated circuits (ASICs), or the like, or any combination thereof. In one embodiment, method 1200 may be performed by the processing devices and the associated algorithms, e.g., as described in conjunction with FIG. 1. In embodiments, method 1200 is performed by processing logic comprising hardware, software, firmware, or a combination thereof. In certain embodiments, method 1200 may be performed by a single processing thread. Alternatively, method 1200 may be performed by two or more processing threads, each thread executing one or more individual functions, routines, subroutines, or operations of the method. In an illustrative example, the processing threads implementing method 1200 may be synchronized (e.g., using semaphores, critical sections, and/or other thread synchronization mechanisms). Alternatively, the processing threads implementing method 1200 may be executed asynchronously with respect to each other. Therefore, while FIG. 12 and the associated descriptions list the operations of method 1200 in a certain order, in some embodiments, at least some of the described operations may be performed in parallel and/or in a different order. In some embodiments, one or more operations of method 1200 is not performed.

At block 1202, processing logic can identify a plurality of reference points within one or more 3D dental models of a patient. In some embodiments, the one or more 3D dental models can include a first 3D dental model of the upper jaw of the patient and a second 3D dental model of the lower jaw of the patient. In some embodiments, the plurality of reference points can include a first reference point on the first 3D dental model of the upper jaw, and a second reference point on the second 3D dental model of the lower jaw. In some embodiments, the second 3D dental model can correspond to an initial stage of a treatment plan for the patient, an intermediary stage of the treatment plan for the patient, or a final stage of the treatment plan for the patient. In some embodiments, the treatment plan for the patient can be an orthodontic treatment plan, such as an aligner orthodontic treatment plan.

In some embodiments, each of the plurality of reference points can correspond to a point on a tooth of the patient. Example reference points are described with respect to FIG. 1. In some embodiments, at least a subset of the plurality of reference points can be selected and/or identified by a user. Processing logic can receive user input (e.g., via the UI) selecting at least a subset of the plurality of reference points. For example, the user can select one or more of the reference points from a dropdown menu, and/or by clicking on a visualization of the dental model displayed in the UI at a location corresponding to the desired reference point. In some embodiments, at least a subset of the plurality of reference points is predetermined.

In some embodiments, processing logic can perform image processing on a first 3D dental model of the one or more 3D dental models to determine a location of at least one of the plurality of reference points within the first 3D dental model. For example, the transverse treatment planning component 112 can implement feature detection, feature matching, and/or georeferencing to identify the coordinates in the 3D dental model corresponding to the reference points. In some embodiments, processing logic can provide the one or more 3D dental models as input to an AI model that provides, as output, a plurality of indications. Each indication of the plurality of indications can represent a location of a reference point within one of the one or more 3D dental models.

In some embodiments, a first tooth measurement of the one or more tooth measurements can represent a distance between a first reference point on a first tooth and a second reference point on a second tooth of the one or more 3D dental models of the patient. In some embodiments, the first reference point can correspond to a tooth cusp and the first tooth can correspond to an upper-left molar of the upper jaw of the patient, and the second reference point can correspond to a second tooth cusp and the second tooth can correspond to a lower-left first molar of the lower jaw. Thus, in this embodiment, the first tooth measurement can represent the distance between a tooth cusp of the upper-left first molar and the tooth cusp of the lower-left first molar. In some embodiments, the first referent point can represent a first midpoint between a lingual-distal cusp and a lingual-mesial cusp, and the second reference point can represent a second midpoint between a buccal-distal cusp and a buccal-mesial cusp.

In some embodiments, the first reference point can correspond to a tooth cusp and the first tooth can correspond to an upper-right molar of the upper jaw of the patient, and the second reference point can correspond to a second tooth cusp and the second tooth can correspond to a lower-right first molar of the lower jaw. Thus, in this embodiment, the first tooth measurement can represent the distance between a tooth cusp of the upper-right first molar and the tooth cusp of the lower-right first molar.

At block 1204, processing logic can determine one or more tooth measurements based on the plurality of reference points. Examples of tooth measurements are described with respect to FIG. 1. At block 1206, processing logic can determine, based on the one or more tooth measurements, a recommended palatal expansion amount for a palatal expansion treatment plan for the patient.

In some embodiments, the one or more 3D dental models can include one or more first 3D dental models of the upper jaw of the patient, and a second 3D dental model of the lower jaw of the patient. To determine the recommended palatal expansion amount, processing logic can identify a plurality of treatment stages of the palatal expansion treatment plan. Each treatment stage of the plurality of treatment stages can correspond to one of the one or more first 3D dental models of the upper jaw. Each tooth measurement of the one or more tooth measurements can correspond to a treatment stage of the plurality of treatment stages.

Processing logic can identify a minimum tooth measurement of the one or more tooth measurements. Processing logic can identify a first treatment stage of the plurality of treatment stages. The first treatment stage can correspond to the minimum tooth measurement, and the recommended palatal expansion amount can be based on the first treatment stage.

In some embodiments, to determine the one or more tooth measurements, processing logic can determine, for each treatment stage of the plurality of treatment stages, a first distance between a first reference point on a right side of the upper jaw and a second reference point on the right side of the lower jaw, and a second distance between a third reference point on a left side of the upper jaw and a fourth reference point on the left side of the lower jaw. In some embodiments, the first reference point can represent a midpoint between a first landmark on a first portion of a first 3D dental model representing the upper jaw and a second landmark on the first portion o the first 3D dental model representing the upper jaw. In some embodiments, the second reference point can represent a second midpoint between a third landmark on a second portion of a second 3D dental model representing the lower jaw and a fourth landmark on the second portion of the second 3D dental model representing the lower jaw. The first portion and/or the second portion can correspond to a tooth, or to a location where a tooth is expected to be located. In some embodiments, the first portion and/or the second portion can correspond to an object within the mouth. As an example, the first portion can represent an upper-right first molar, the second portion can represent a lower-right first molar. That is, in some embodiments, the first portion can correspond to the second portion (e.g., the first portion and the second portion can each represent a first molar of the patient). The landmarks can represent an anatomical feature of a tooth (such as a tooth cusp, tooth crown, etc.). For example, the first landmark can represent a lingual-distal tooth cusp, the second landmark can represent a lingual-mesial tooth cusp, the third landmark can represent a buccal-distal tooth cusp, and the fourth landmark can represent buccal-mesial tooth cusp.

In some embodiments, the third reference point can represent a third midpoint between a fifth landmark on a third portion of the first 3D dental model representing the upper jaw and a sixth landmark on the third portion of the first 3D dental model representing the upper jaw. The fourth reference point can represent a fourth midpoint between a seventh landmark on a fourth portion of the second 3D dental model representing the lower jaw of the patient and an eighth landmark of the fourth portion of the second 3D dental model representing the lower jaw. The third portion can correspond to the fourth portion. The third portion and/or the fourth portion can correspond to a tooth, or to a location where a tooth is expected to be located. In some embodiments, the third portion and/or the fourth portion can correspond to an object within the mouth. As an example, the third portion can represent an upper-left first molar and the fourth portion can represent a lower-left first molar. The landmarks can represent an anatomical feature of a tooth (such as a tooth cusp, tooth crown, etc.). For example, the fifth landmark can represent a lingual-distal tooth cusp, the sixth landmark can represent a lingual-mesial tooth cusp, the seventh landmark can represent a buccal-distal tooth cusp, and the eighth landmark can represent buccal-mesial tooth cusp.

In some embodiments, to determine the one or more tooth measurements, the processing logic can, for each treatment stage of the plurality of treatment stages and for each side of a right side and a left side, perform the following operations: generate a first occlusal plane for a first side of the second 3D dental model of the patient. The first occlusal plane can extend for a first buccal cusp of a premolar tooth along a second buccal cusp of a molar tooth. Processing logic can generate a second occlusal plane for the second 3D dental model of the patient. The second occlusal plane can extend from a tooth crown center of a first-lower molar of the second 3D dental model along a first direction (e.g., mesial-distal direction). Processing logic can generate a ray intersecting the first occlusal plane and the second occlusal plane. Processing logic can identify a first midpoint between a lingual-distal cusp and a lingual-mesial cusp of the first-upper molar of the first 3D dental model and a second midpoint between a buccal-distal cusp and a buccal-mesial cusp of the first-lower molar of the second 3D dental model. The first 3D dental model and the second 3D dental model correspond to a particular treatment stage of the plurality of treatment stages. Processing logic can project the first midpoint and the second midpoint to the ray. Processing logic can calculate a distance between the first midpoint and the second midpoint projected onto the ray. The one or more tooth measurements can include the distance between the first midpoint and the second midpoint.

In some embodiments, to determine the recommended palatal expansion among, processing logic can provide, as input to an AI model, the one or more tooth measurements. The AI model can provide, as output, the recommended palatal expansion amount.

At block 1208, processing logic can provide, to a user device (e.g., computing device 160 and/or oral state capture system 110 of FIG. 1) the recommended palatal expansion amount.

In some embodiments, processing logic can provide, for display in a UI of the user device, a representation of the one or more 3D dental models. The representation can include an indicator of a first reference point of the plurality of reference points. An example of a UI that includes at one indicator of a reference point is described with respect to FIGS. 8, 10. In some embodiments, processing logic can provide, for display in the UI of the user device, the recommended palatal expansion amount, a number of expanders to achieve the recommended palatal expansion amount, and/or at least one of the tooth measurements. An example of such a UI is described with respect to FIGS. 9-10.

In some embodiments, processing logic can provide instructions to fabricate one or more dental appliances to implement the palatal expansion treatment plan. In some embodiments, the dental appliances can be fabricated by fabrication machine(s) 170 of FIG. 1. In some embodiments, the one or more dental appliances can include a palatal expander, and/or one or more incremental palatal expanders. In some embodiments, the one or more dental appliances can be 3D printed incremental palatal expanders.

FIG. 13 illustrates a flow diagram of an example method 1300 for visualizing a cross-sectional plane and reference points for PE treatment, in accordance with some embodiments of the present disclosure. Method 1300 may be performed by a processing device that may include hardware, software, or a combination of both. The processing device may include one or more central processing units (CPUs), graphics processing units (GPUs), field-programmable gate arrays (FPGAs), application-specific integrated circuits (ASICs), or the like, or any combination thereof. In one embodiment, method 1300 may be performed by the processing devices and the associated algorithms, e.g., as described in conjunction with FIG. 1. In embodiments, method 1300 is performed by processing logic comprising hardware, software, firmware, or a combination thereof. In certain embodiments, method 1300 may be performed by a single processing thread. Alternatively, method 1300 may be performed by two or more processing threads, each thread executing one or more individual functions, routines, subroutines, or operations of the method. In an illustrative example, the processing threads implementing method 1300 may be synchronized (e.g., using semaphores, critical sections, and/or other thread synchronization mechanisms). Alternatively, the processing threads implementing method 1300 may be executed asynchronously with respect to each other. Therefore, while FIG. 13 and the associated descriptions list the operations of method 1300 in a certain order, in some embodiments, at least some of the described operations may be performed in parallel and/or in a different order. In some embodiments, one or more operations of method 1300 is not performed.

At block 1302, processing logic can identify one or more 3D dental models of a patient. The one or more 3D dental models can correspond to a palatal expansion treatment plan for the patient. In some embodiments, at least one of the one or more 3D dental models are based on scan data of the patient, wherein the scan data is generated using one or more imaging modalities comprising at least one of a cone beam computed tomography (CBCT) scan, a radiograph, a computed tomography (CT) scan, an intraoral scan, a color image, a near-infrared (NIR) image, or an image generated using fluorescence imaging

At block 1304, processing logic can determine a 2D cross-section of each of the one or more 3D dental models that intersects a first portion of each of the one or more 3D dental models at a corresponding first location.

In some embodiments, the first portion can correspond to a 3D object of the one or more 3D dental models. A corresponding first location can correspond to a first reference point of the 3D object, and the first reference point can correspond to at least one of a surface point or an internal point of the 3D object. In some embodiments, the first portion can correspond to a tooth of the patient. In some embodiments, the 2D cross-section of the one or more 3D dental models illustrates both sides (e.g., the right side and the left side) of an upper jaw of the patient and both sides (e.g., the right side and the left side) of a lower jaw of the patient. In some embodiments, the 2D cross-section of the one or more 3D dental models is displayed in color. In some embodiments, the 2D cross-section of the one or more 3D dental models is displayed in black-and-white.

At bock 1306, processing logic can provide, for display on a UI of a user device (e.g., computing device 160, oral state capture system 110 FIG. 1), the 2D cross-section of each of the one or more 3D dental models. In some embodiments, the user device corresponds to a patient device, a doctor device, or a scanning device. An example UI illustrating the 2D cross-section is described with respect to FIGS. 9, 10. In some embodiments, processing logic can output an occlusal-view photograph at a first region of the UI, and can output the 2D cross-section at a second region of the UI. In some embodiments, the occlusal-view photograph can include at least a first portion and an indication of a location of the 2D cross-section. In some embodiments, the 2D cross-section represents a side-view of the first portion of the 3D dental model. An example UI that includes an occlusal-view photograph and the 2D cross-section is described with respect to FIGS. 9-10.

In some embodiments, processing logic can provide, for display on the UI of the user device, the one or more 3D dental models and an indication of the corresponding first location on the one or more 3D dental models. An example of such a UI is described with respect to FIGS. 9-10.

At block 1308, processing logic can determine one or more palatal expansion measurements at the 2D cross-section for an amount of palatal expansion associated with the palatal expansion treatment plan at a stage of treatment.

In some embodiments, a first palatal expansion measurement is associated with a first reference point of a plurality of reference points. The first reference point can correspond to the first portion. For example, the first reference point can be the midpoint between the upper cusp lingual distal and the upper cusp lingula mesial. Processing logic can determine a first location of the first reference point on the 2D cross-section, and can provide, for display on the UI of the user device, a first indicator representing the first location of the first reference point on the 2D cross-section. An example UI illustrating the first indicator is described with respect to FIG. 10. In some embodiments, processing logic can provide, display on the UI of the user device, a second indicator representing the first location of the first reference on the 3D dental model. An example UI illustrating the second indicator is described with respect to FIG. 10.

In some embodiments, processing logic can receive a user input selecting at least a subset of the plurality of reference points. For example, the user can select one or more of the reference points from a dropdown menu, and/or by clicking on a visualization of the dental model displayed in the UI at a location corresponding to one of the reference points. In some embodiments, one or more of the plurality of reference points can be predetermined.

In some embodiments, processing logic can perform image processing on a first 3D dental model of the one or more 3D dental models to determine a location of at least one of the plurality of reference points within the first 3D dental model. For example, the transverse treatment planning component 112 can implement feature detection, feature matching, and/or georeferencing to identify the coordinates in the 3D dental model corresponding to the reference points. In some embodiments, processing logic can provide the one or more 3D dental models as input to AI model. The AI model can provide, as output, a plurality of indications. Each indication can represent a location of a reference point within one of the one or more 3D dental models.

At block 1310, processing logic can provide, for display on the UI of the user device, the one or more palatal expansion measurements.

In some embodiments, processing logic can receive user input. For example, the user can select a location of a tooth at which to the 2D cross-section is to intersect the tooth (e.g., using the sliding bar 910 of FIG. 9, and/or by interacting with the dotted line 906 of FIG. 9). In response to receiving the user input, processing logic can generate a second 2D cross-section to intersect each of the one or more 3D dental models at a second location. Processing logic can provide, for display on the UI of the user device, the second 2D cross-section of each of the one or more 3D dental models at the second location. Processing logic can determine one or more second palatal expansion measurements at the second 2D cross-section for a second amount of palatal expansion associated with the palatal expansion treatment plan at a second stage of treatment. Processing logic can provide, for display on the UI of the user device, the one or more second palatal expansion measurements.

In some embodiments, processing logic can provide a ruler tool in the UI of the user device. The ruler tool can provide a visualization of at least one of the one or more palatal expansion measurements. An example of a UI that that includes a ruler tool is described with respect to FIG. 11.

Control of the various systems described in this specification, or portions of them, can be implemented in a digital computer program product that includes instructions that are stored on one or more non-transitory machine-readable storage media, and that are executable on one or more digital processing devices. The systems described in this specification, or portions of them, can each be implemented as an apparatus, method, or system that may include one or more digital processing devices and memory to store executable instructions to perform the operations described in this specification.

While this specification contains many specific embodiment details, these should not be construed as limitations on the scope of what may be claimed, but rather as descriptions of features that may be specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or variation of a sub-combination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described above should not be understood as requiring such separation in all embodiments, and it should be understood that the described program components and systems can generally be integrated together in a single software product or packaged into multiple software products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In some cases, multitasking and parallel processing may be advantageous.

What is claimed:

1. A method comprising:
receiving scan data of a craniofacial structure of a patient;
providing the scan data as input to an artificial intelligence (AI) model trained to provide a recommended palatal expansion treatment plan for the patient;
receiving, as output from the AI model, the recommended palatal expansion treatment plan; and
providing, to a user device, the recommended palatal expansion treatment plan.

2. The method of claim 1, wherein the recommended palatal expansion treatment plan comprises a plurality of treatment stages, each associated with one or more dental appliances that are usable to implement the palatal expansion treatment plan, and wherein a visual representation of a first dental appliance of the one or more dental appliances is provided for display in a user interface of the user device.

3. The method of claim 2, wherein the first dental appliance corresponds to a treatment stage of the plurality of treatment stages.

4. The method of claim 2, wherein at least the treatment stage of the palatal expansion treatment plan is provided for display in a first portion of a user interface (UI) and at least the first dental appliance of the one or more dental appliances is provided for display in a second portion of the UI.

5. The method of claim 2, wherein a visualization of a dentition of the patient and the visualization of the first dental appliance worn over the dentition are output to a user interface (UI).

6. The method of claim 1, wherein the recommended palatal expansion treatment plan comprises at least one of: an amount of expansion of a palate of the patient or a number of expanders in the recommended palatal expansion treatment plan.

7. The method of claim 1, further comprising:
identifying one or more values corresponding to palatal expansion treatment plan parameters; and
providing, as additional input to the AI model, the one or more values.

8. The method of claim 1, further comprising:
providing, as input to a second AI model, the scan data of the craniofacial structure of the patient and the recommended palatal expansion treatment plan, wherein the second AI model is trained to provide a predicted outcome of the craniofacial structure of the patient;
receiving, as output from the second AI model, the predicted outcome of the craniofacial structure of the patient;
generating a visualization of the predicted outcome of the craniofacial structure; and
providing, for display in a user interface of the user device, the visualization of the predicted outcome.

9. The method of claim 8, further comprising:
identifying one or more parameters corresponding to the recommended palatal expansion treatment plan; and
providing the one or more parameters as additional input to the second AI model.

10. The method of claim 9, wherein the one or more parameters comprise at least one of an amount of expansion, a vertical clearance measurement of a palatal expander of the recommended palatal expansion treatment plan, a placement of an attachment on a tooth of a dentition of the patient, a first identification of at least one tooth covered by the expander, a second identification of a corresponding part of a palate of the patient covered by the expander, or a second amount of transverse force applied by the expander.

11. The method of claim 8, wherein the predicted outcome includes at least one of an amount of expansion of a palate of the patient, a predicted placement of at least one tooth in a dentition of the patient, or a predicted shape of the palate of the patient.

12. The method of claim 1, wherein the recommended palatal expansion treatment plan comprises a series of breadths corresponding to a progressive expansion of a palate of the patient from an initial breadth toward a target breadth.

13. The method of claim 1, wherein the recommended palatal expansion treatment plan comprises one or more dental appliances to implement the recommended palatal expansion treatment plan.

14. The method of claim 13, wherein the one or more dental appliances comprises a palatal expander.

15. The method of claim 13, wherein the one or more dental appliances comprises one or more incremental palatal expanders.

16. The method of claim 13, wherein the one or more dental appliances comprise one or more 3D printed incremental palatal expanders.

17. The method of claim 1, further comprising providing instructions to fabricate a series of palatal expanders corresponding to the palatal expansion treatment plan.

18. The method of claim 1, wherein the user device corresponds to a patient device, a doctor device, or a scanning device.

19. A system comprising:
a memory; and
a processing device to execute instructions from the memory to:
receive scan data of a craniofacial structure of a patient;
provide the scan data as input to an artificial intelligence (AI) model trained to provide a recommended palatal expansion treatment plan for the patient;
receive, as output from the AI model, the recommended palatal expansion treatment plan; and
provide, to a user device, the recommended palatal expansion treatment plan.

20. A non-transitory computer-readable storage medium comprising instructions that, when executed by a processing device, cause the processing device to:
receive scan data of a craniofacial structure of a patient;
provide the scan data as input to an artificial intelligence (AI) model trained to provide a recommended palatal expansion treatment plan for the patient;
receive, as output from the AI model, the recommended palatal expansion treatment plan; and
provide, to a user device, the recommended palatal expansion treatment plan.
